(19) United States
(12) Patent Application Publication
(10) Pub. No.: US 2010/0099746 A1
(43) Pub. Date: Apr. 22, 2010

(54) NOVEL NUCLEIC ACID

(75) Inventors: Yoji Yamada, Sunto-gun (JP); Tatsuya Miyazawa, Machida-shi (JP); Tetsuo Yoshida, Machida-shi (JP); Haruo Nakano, Machida-shi (JP); Kyoko Kosaka, Machida-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KYOWA HAKKO KIRIN CO., LTD., Tokyo (JP)

(21) Appl. No.: 12/519,960

(22) PCT Filed: Dec. 18, 2007
(86) PCT No.: PCT/IB07/04005

§ 371 Date: Jun. 18, 2009

(30) Foreign Application Priority Data
Dec. 18, 2006 (JP) 2006-339997

(51) Int. Cl. A61K 31/7088; C07H 21/04; C12N 15/63; C12N 5/10; C12Q 1/02; C12Q 1/68; A61P 35/00

(52) U.S. Cl. 514/44 R; 536/23.1; 435/320.1; 435/325; 435/29; 435/6

(57) ABSTRACT

The present invention provides a nucleic acid such as a micro-RNA or a micro-RNA precursor, having a novel sequence. The nucleic acid of the present invention is useful for detecting the expression or a mutation of a micro-RNA, separating cells, suppressing the expression of a gene having a target sequence, screening for a substance that promotes or suppresses a function of a micro-RNA, and diagnosing or treating a disease caused by a mast cell abnormality, a disease caused by an abnormality of mesenchymal stem cell proliferation or differentiation, cancers, and a disease caused by abnormal proliferation of cells, tissue hyperplasia or the like.

### TECHNICAL FIELD

The present invention relates to a novel nucleic acid, a method of expressing or suppressing the nucleic acid, and a diagnostic reagent or a therapeutic agent comprising the nucleic acid.

### BACKGROUND ART

A micro-RNA (miRNA), which is one type of nucleic acid, is a small non-coding single-stranded RNA of about 22 nucleotides that is not translated into a protein, and has been confirmed as being present in many types in organisms, including humans (non-patent documents 1 and 2).

A micro-RNA is produced from a gene transcribed to a single or clustered micro-RNA precursor. Specifically, first, a primary-microRNA (pri-miRNA), which is a primary transcript, is transcribed from the gene, then, in stepwise processing from the pri-miRNA to a mature type micro-RNA, a precursor-microRNA (pre-miRNA) of about 70 nucleotides having a characteristic hairpin structure is produced from the pri-miRNA. Furthermore, the mature type micro-RNA is produced from the pre-miRNA by Dicer-mediated processing (non-patent document 3).

A mature type micro-RNA is thought to be involved in the post-transcriptional control of gene expression by complementarily binding to a target mRNA to suppress the translation of the mRNA, or to degrade the mRNA. As of May 2006, in the micro-RNA database miRBase (http://microrna.sanger.ac.uk/), 455 species of micro-RNAs were registered for humans, and 3685 species for all organisms. Of the micro-RNAs expressed in mammals, including humans, only some have their physiological functions elucidated to date, including miR-181, which is involved in hematopoietic lineage differentiation (non-patent document 4), miR-375, which is involved in insulin secretion (non-patent document 5), and the like; many have their bioactivities unclarified. However, studies using nematodes or Drosophila have shown that micro-RNAs play various important roles in the development and differentiation in organisms, and a report of the relation to human diseases has been presented suggesting a profound relation to cancers (non-patent document 6).

For identification of micro-RNAs, there are a method wherein a low-molecular RNA is cloned from a cell, a method wherein bioinformatics is applied to genome sequence information, and the like. Registration of any micro-RNA in the miRBase requires both information on the expression and information on the biosynthesis and structure; a structural prediction from genome sequence information only does not suffice approval as a micro-RNA (non-patent document 7).

Mast cells are known to be activated by various stimuli to undergo degranulation and release or produce many inflammatory mediators (non-patent documents 8 to 10). For example, it is known that when an antigen is recognized by a mast cell, histamine and tryptase are quickly released upon degranulation, and chemical mediators such as prostaglandin D2 (PGD2), leukotriene (LT), and platelet activation factor (PAF), various chemokines such as macrophage inflammatory protein (MIP)-1α, and various cytokines such as granulocyte macrophage colony stimulation factor (GM-CSF) are newly synthesized and released. Regarding major basic proteins, which are cytotoxic proteins that have been thought to be produced by eosinophils, it has recently been shown that in the case of humans, they are produced in large amounts by mast cells (non-patent document 11).

Hence, mast cells are thought to play major roles in the pathogenesis of various allergic diseases; therefore, it is thought that by controlling a function of mast cells, treatment of allergic diseases is possible.

However, it is known that rodent mast cells and human mast cells have different reactivities to drugs (non-patent document 9). Specifically, sodium cloroglycate, which is used as a suppressant of inflammatory mediator release, remarkably suppresses the IgE-dependent release of inflammatory mediators in rat abdominal mast cells, but the action thereof on human mast cells is not potent (non-patent document 12). Azelastine hydrochloride, at high concentrations, suppresses the release of histamine, PGD2, and LT and production of GM-CSF and MIP-1α, from human mast cells in culture, but none of these activities are potent. Suplatast tosilate, which is used as an anti-cytokine drug, exhibits inflammatory mediator release suppressive action on rat mast cells, but lacks action on human mast cells (non-patent document 12).

As a result of a comparison of genes expressed in human mast cells and mouse mast cells, it is known that the genes expressed by various stimuli do not always agree (non-patent document 13).

Regarding micro-RNAs, micro-RNAs expressed in mouse bone marrow derived mast cells have been reported (non-patent document 14), but no relationship is known between a micro-RNA and a function of mast cells. No report is available on a micro-RNA expressed in human mast cells; taking into account the above-described interspecific differences between humans and mice, it is difficult to predict information on the expression of micro-RNAs in human mast cells on the basis of information on the expression of micro-RNAs in mouse mast cells.

Mesenchymal stem cells are present in mammalian bone marrow, fat tissue, umbilical blood and the like, and are known as multipotent stem cells that differentiate into adipocytes, chondrocytes, osteocytes and the like. Mesenchymal stem cells, because of the multipotency thereof, are attracting attention as graft materials for regenerative medicine for many tissues, including bones, cartilage, tendons, muscles, fat, and periodontal tissue (non-patent document 15).

Mesenchymal stem cells can be differentiated into particular cells in vitro by the addition of a drug, a cytokine and the like; for example, differentiation into adipocytes can be induced by allowing 1-methyl-3-isobutylxanthine, dexamethasone, insulin and indomethacin to act, and differentiation into osteoblasts can be induced by allowing dexamethasone, β-glycerol phosphate, and ascorbic acid to act (non-patent document 16). However, details of the molecular mechanisms in these differentiation processes are unknown. From the results of gene expression analyses on gene-knockout mice and in the differentiation stage, it is known that differentiation into adipocytes is mediated by the PPARγ and C/EBP families, and that during osteoblast differentiation, the expression of genes such as Cbfa1/Runx2 and Osterix is involved (non-patent document 17); however, the mechanisms of differentiation from mesenchymal stem cells cannot be explained solely on the basis of these genes, and artificial control of the differentiation and proliferation has not been realized. Also, no micro-RNA is known to act on the differentiation and proliferation of mesenchymal stem cells.

Because micro-RNAs are involved in the control of the expression of a wide variety of genes, abnormalities of micro-RNAs are supposed to be involved in various human diseases. Particularly in cancers, research has been advanced; it has been reported that in many cancers, the expression of micro-RNAs differs from that in normal tissues, that classification of cancers is enabled by expression profile analyses of micro-RNAs, and the like (non-patent document 18). It is also known that about half of the human micro-RNAs that have been found so far are present in chromosome aberrations or fragile portions of chromosomes known in human cancers (non-patent document 19). Examples of relationships between cancers and micro-RNAs that have been reported so far include the finding that the miR-15a/miR-16 cluster is contained in chromosome 13q14, which is deleted in B cell chronic lymphatic leukemia (B-CLL), the deletion being supposed to be a cause of B-CLL (non-patent document 20), the finding that in lung cancer, the expression of Let-7, which is a micro-RNA, is decreased, one of the targets thereof being Ras, which is known as a carcinogenic gene (non-patent documents 21 and 22), and the like. Many micro-RNAs have their expression decreased in cancer cells; conversely, however, there are some micro-RNAs with gene amplification or overexpression in cancers. For example, in regions where gene amplification is seen in malignant lymphoma, a cluster consisting of six species of micro-RNAs (miR-17-92) is present; it has been reported that when this miRNA cluster gene is forcibly expressed in a mouse model of human B cell lymphoma, the onset of lymphoma is promoted (non-patent document 23). It has also been shown that a gene called BIC, which does not encode a protein and has been regarded as a candidate for the cancer gene that is overexpressed in Hodgkin lymphoma, encodes miR-155 (non-patent document 24).

As stated above, relationships between cancers and micro-RNAs have recently been reported in many cases, but most of them show expression abnormalities in cancer cells; there are only a few studies showing a function of a micro-RNA, including a report that the proliferation of a cancer cell line was inhibited by forcibly expressing Let-7 in a lung cancer cell line (non-patent document 25) and the like. Currently, no report is available that cancer growth was suppressed in an animal model by administering a micro-RNA or a precursor thereof, or an antisense oligonucleotide thereof, from outside the body to increase or decrease the expression of the micro-RNA.
non-patent document 1: Science, 294, 853-858 (2001)
non-patent document 2: Cell, 113, 673-676 (2003)
non-patent document 3: Nature Reviews Genetics, 5, 522-531 (2004)
non-patent document 4: Science, 303, 83-86 (2004)
non-patent document 5: Nature, 432, 226-230 (2004)
non-patent document 6: Nature Reviews Cancer, 6, 259-269 (2006)
non-patent document 7: RNA, 9, 277-279 (2003)
non-patent document 8: Himan Saibo no Rinsho, ed. Motohito Kurosawa, Sentan Igaku-sha Ltd., p 142 (2001)
non-patent document 9: Himan Saibo no Rinsho, ed. Motohito Kurosawa, Sentan Igaku-sha Ltd., p 559 (2001)
non-patent document 10: Crit. Rev. Immunol., 22, 115-140 (2002)]
non-patent document 11: Blood, 98, 1127-1134 (2001)
non-patent document 12: Clin. Exp. Allergy, 28, 1228-1236 (1998)
non-patent document 13: Blood, 100, 3861-3868 (2002)
non-patent document 14: Genome Biology, 6, R71 (2005)
non-patent document 15: Idenshi Igaku, vol. 4, p. 58-61 (2000)
non-patent document 16: Science, 284, 143-147 (1999)
non-patent document 17: Jikken Igaku, vol. 20, p. 2459-2464 (2000)
non-patent document 18: Nature, 435, 839-843 (2005)
non-patent document 19: Proc. Natl. Acad. Sci. USA, 101, 2999-3004 (2004)
non-patent document 20: Proc. Natl. Acad. Sci. USA, 99, 15524-15529 (2002)
non-patent document 21: Cancer Research, 64, 3753-3756, (2004)
non-patent document 22: Cell, 120, 635-647, (2005)
non-patent document 23: Nature, 435, 823-833 (2005)
non-patent document 24: Proc. Natl. Acad. Sci. USA, 102, 3627-3632 (2005)
non-patent document 25: Cancer Research, 64, 3753-3756, (2004)

### DISCLOSURE OF THE INVENTION

### Problems to be Solved by the Invention

It is expected that by identifying micro-RNAs expressed in various human organs, and analyzing the functions thereof to elucidate their relations to diseases, new therapeutic agents and diagnostic reagents will be developed.

Finding a micro-RNA that acts in mast cells is expected to lead to the functional elucidation of differentiation, degranulation, inflammatory mediator production, cytokine production, chemokine production and the like in mast cells, and to lead to the development of methods of isolation, cultivation, differentiation control, degranulation control, inflammatory mediator production control, cytokine production control, and chemokine production control for mast cells, as well as new therapies for allergic diseases and the like based thereon.

In addition, finding a micro-RNA that acts in mesenchymal stem cells is expected to lead to the functional elucidation of differentiation and proliferation of mesenchymal stem cells, and to lead to the development of a method of controlling the differentiation from mesenchymal stem cells to particular cells and a new therapy based on differentiation control.

Furthermore, finding a micro-RNA that causes cancer cell proliferation or suppression is expected not only to help to understand the mechanisms of carcinogenesis, but also to lead to the development of diagnostic reagents and therapeutic agents for human cancers, and new diagnostic methods and therapies for cancers based thereon. Furthermore, regarding diseases other than cancers, the same is expected to contribute to the development of diagnostic reagents and therapeutic agents for diseases caused by abnormal proliferation of cells, tissue hyperplasia and the like, such as arteriosclerosis, rheumatoid arthritis, prostatic hyperplasia, blood vessel restenosis after percutaneous transvascular coronary angioplasty, fibroid lung, glomerulonephritis, and autoimmune diseases, and diagnostic methods and therapies based thereon.

It is an object of the present invention to acquire micro-RNAs, and to provide nucleic acids that are useful in isolation, cultivation, differentiation control, degranulation control, inflammatory mediator production control, cytokine production control, and chemokine production control in mast cells, diagnosis and treatment of allergic diseases, control of mesenchymal stem cell differentiation and proliferation, control of cancer cell differentiation and proliferation, and diagnosis and treatment of diseases such as cancers, as well as methods of utilizing the same.

### Means of Solving the Problems

The present invention relates to (1) to (64) below.
(1) A nucleic acid consisting of the nucleotide sequence of any one of SEQ ID NOs:1 to 1336.
(2) A nucleic acid consisting of a nucleotide sequence having an identity of 90% or more to the nucleotide sequence of any one of SEQ ID NOs:1 to 1336.
(3) A nucleic acid that hybridizes under stringent conditions with a strand complementary to a nucleic acid consisting of the nucleotide sequence of any one of SEQ ID NOs:1 to 1336.
(4) A nucleic acid comprising the nucleic acid described in any one of (1) to (3).
(5) A nucleic acid consisting of the nucleotide sequence of any one of SEQ ID NOs:1337 to 2851.
(6) A nucleic acid consisting of a nucleotide sequence having an identity of 80% or more to the nucleotide sequence of any one of SEQ ID NOs:1337 to 2851.
(7) A nucleic acid that hybridizes under stringent conditions with a strand complementary to a nucleic acid consisting of the nucleotide sequence of any one of SEQ ID NOs:1337 to 2851.
(8) A nucleic acid consisting of a nucleotide sequence complementary to the nucleic acid described in any one of (1) to (7).
(9) A double-stranded nucleic acid consisting of the nucleic acid described in any one of (1) to (7) and a nucleic acid consisting of a nucleotide sequence complementary to the nucleotide sequence of the nucleic acid.
(10) A vector which expresses the nucleic acid described in any one of (1) to (9).
(11) A method of detecting the expression or mutation of the nucleic acid described in any one of (1) to (9), comprising using the nucleic acid described in any one of (1) to (9).
(12) A method for screening a substance that promotes or suppresses the expression or function of the nucleic acid described in any one of (1) to (9), comprising using the nucleic acid described in any one of (1) to (9).
(13) A method of separating a cell that expresses the nucleic acid described in any one of (1) to (9), comprising using the nucleic acid described in any one of (1) to (9).
(14) A method of suppressing the expression of a target gene of the nucleic acid described in any one of (1) to (9), comprising using the nucleic acid described in any one of (1) to (9).
(15) A diagnostic reagent or a therapeutic agent for a disease caused by a mast cell abnormality, comprising the nucleic acid described in any one of (1) to (9) as an active ingredient.
(16) A diagnostic reagent or a therapeutic agent for a disease caused by a mast cell abnormality, comprising a substance that promotes or suppresses the expression or function of the nucleic acid described in any one of (1) to (9) as an active ingredient.
(17) A method for screening a diagnostic reagent or a therapeutic agent for a disease caused by a mast cell abnormality, wherein promotion or suppression of the expression or function of the nucleic acid described in any one of (1) to (9) serves as an index.
(18) A diagnostic reagent or a therapeutic agent for a disease caused by a mast cell abnormality, comprising a substance that suppresses the expression of a target gene of the nucleic acid described in any one of (1) to (9) as an active ingredient.
(19) A method for screening a diagnostic reagent or a therapeutic agent for a disease caused by a mast cell abnormality, wherein suppression of the expression of a target gene of the nucleic acid described in any one of (1) to (9) serves as an index.
(20) A cell incorporating the nucleic acid or vector described in any one of (1) to (10).
(21) A mast cell degranulation promoter comprising the nucleic acid described in any one of (1) to (4) wherein the SEQ ID NO: is any one of 1, 2, 3, 8, 14, 20, 22, 25, 32 and 36, as an active ingredient.
(22) A mast cell degranulation promoter comprising the nucleic acid described in any one of (5) to (7) wherein the SEQ ID NO: is any one of 1337, 1338, 1339, 1352, 1363, 1371, 1373, 1377, 1386 and 1390, as an active ingredient.
(23) A mast cell degranulation suppressant comprising a nucleic acid consisting of a nucleotide sequence complementary to the nucleic acid described in (21) or (22) as an active ingredient.
(24) A mast cell degranulation promoter or degranulation suppressant comprising a double-stranded nucleic acid consisting of the nucleic acid described in (21) or (22) and a nucleic acid consisting of a nucleotide sequence complementary to the nucleotide sequence of the nucleic acid, as an active ingredient.
(25) A mast cell degranulation promoter or degranulation suppressant comprising a substance that promotes or suppresses the expression or function of the nucleic acid described in (21) or (22) as an active ingredient.
(26) A method for screening a mast cell degranulation promoter or degranulation suppressant, wherein promotion or suppression of the expression or function of the nucleic acid described in (21) or (22) serves as an index.
(27) A mast cell degranulation promoter or degranulation suppressant comprising a substance that suppresses or promotes the expression of a target gene of the nucleic acid described in (21) or (22) as an active ingredient.
(28) A method for screening a mast cell degranulation promoter or degranulation suppressant, wherein suppression or promotion of the expression of a target gene of the nucleic acid described in (21) or (22) serves as an index.
(29) A diagnostic reagent or a therapeutic agent for a disease caused by an abnormality of mesenchymal stem cell proliferation and/or differentiation, comprising the nucleic acid described in any one of (1) to (4) wherein the SEQ ID NO: is any one of 1, 8, 21 and 36, as an active ingredient.
(30) A diagnostic reagent or a therapeutic agent for a disease caused by an abnormality of mesenchymal stem cell proliferation and/or differentiation, comprising the nucleic acid described in any one of (5) to (7) wherein the SEQ ID NO: is any one of 1337, 1352, 1372 and 1390, as an active ingredient.
(31) A diagnostic reagent or a therapeutic agent for a disease caused by an abnormality of mesenchymal stem cell proliferation and/or differentiation, comprising a nucleic acid consisting of a nucleotide sequence complementary to the nucleic acid described in (29) or (30) as an active ingredient.
(32) A diagnostic reagent or a therapeutic agent for a disease caused by an abnormality of mesenchymal stem cell proliferation and/or differentiation, comprising a double-stranded nucleic acid consisting of the nucleic acid described in (29) or (30) and a nucleic acid consisting of a nucleotide sequence complementary to the nucleotide sequence of the nucleic acid as an active ingredient.
(33) A diagnostic reagent or a therapeutic agent for a disease caused by an abnormality of mesenchymal stem cell proliferation and/or differentiation, comprising a substance that promotes or suppresses the expression or function of the nucleic acid described in (29) or (30) as an active ingredient.
(34) A method for screening a diagnostic reagent or a therapeutic agent for a disease caused by an abnormality of mesenchymal stem cell proliferation and/or differentiation, wherein promotion or suppression of the expression or function of the nucleic acid described in (29) or (30) serves as an is index.
(35) A diagnostic reagent or a therapeutic agent for a disease caused by an abnormality of mesenchymal stem cell proliferation and/or differentiation, comprising a substance that suppresses or promotes the expression of a target gene of the nucleic acid described in (29) or (30) as an active ingredient.
(36) A method for screening a diagnostic reagent or a therapeutic agent for a disease caused by an abnormality of mesenchymal stem cell proliferation and/or differentiation, wherein suppression or promotion of the expression of a target gene of the nucleic acid described in (29) or (30) serves as an index.
(37) A mesenchymal stem cell proliferation promoter comprising the nucleic acid described in any one of (1) to (4) wherein the SEQ ID NO: is 1, as an active ingredient.
(38) A mesenchymal stem cell proliferation promoter comprising the nucleic acid described in any one of (5) to (7) wherein the SEQ ID NO: is 1337, as an active ingredient.
(39) A mesenchymal stem cell proliferation suppressant comprising a nucleic acid consisting of a nucleotide sequence complementary to the nucleic acid described in (37) or (38) as an active ingredient.
(40) A mesenchymal stem cell proliferation suppressant comprising the nucleic acid described in any one of (1) to (4) wherein the SEQ ID NO: is any one of 8, 21 and 36, as an active ingredient.
(41) A mesenchymal stem cell proliferation suppressant comprising the nucleic acid described in any one of (5) to (7) wherein the SEQ ID NO: is any one of 1352, 1372 and 1390, as an active ingredient.
(42) A mesenchymal stem cell proliferation promoter comprising a nucleic acid consisting of a nucleotide sequence complementary to the nucleic acid described in (37) or (38) as an active ingredient.
(43) A mesenchymal stem cell proliferation promoter or proliferation suppressant comprising a double-stranded nucleic acid consisting of the nucleic acid described in any one of (37) to (42) and a nucleic acid consisting of a nucleotide sequence complementary to the nucleotide sequence of the nucleic acid, as an active ingredient.
(44) A mesenchymal stem cell proliferation promoter or proliferation suppressant comprising a substance that promotes or suppresses the expression or function of the nucleic acid described in any one of (37) to (42), as an active ingredient.
(45) A method for screening a mesenchymal stem cell proliferation promoter or proliferation suppressant, wherein promotion or suppression of the expression or function of the nucleic acid described in any one of (37) to (42) serves as an index.
(46) A mesenchymal stem cell proliferation promoter or proliferation suppressant comprising a substance that suppresses or promotes the expression of a target gene of the nucleic acid described in any one of (37) to (42) as an active ingredient.
(47) A method for screening a mesenchymal stem cell proliferation promoter or proliferation suppressant, wherein suppression or promotion of the expression of a target gene of the nucleic acid described in any one of (37) to (42) serves as an index.
(48) A diagnostic reagent or a therapeutic agent for a disease caused by a cell proliferation abnormality, comprising the nucleic acid described in any one of (1) to (4) wherein the SEQ ID NO: is any one of 1, 3, 8, 20, 21, 22, 32 and 36, as an active ingredient.
(49) A diagnostic reagent or a therapeutic agent for a disease caused by a cell proliferation abnormality, comprising the nucleic acid described in any one of (5) to (7) wherein the SEQ ID NO: is any one of 1337, 1339, 1352, 1371, 1372, 1373, 1386 and 1390, as an active ingredient.
(50) A diagnostic reagent or a therapeutic agent for a disease caused by a cell proliferation abnormality, comprising a nucleic acid consisting of a nucleotide sequence complementary to the nucleic acid described in (48) or (49) as an active ingredient.
(51) A diagnostic reagent or a therapeutic agent for a disease caused by a cell proliferation abnormality, comprising a double-stranded nucleic acid consisting of the nucleic acid described in (48) or (49) and a nucleic acid consisting of a nucleotide sequence complementary to the nucleic acid as an active ingredient.
(52) A diagnostic reagent or a therapeutic agent for a disease caused by a cell proliferation abnormality, comprising a substance that promotes or suppresses the expression or function of the nucleic acid described in (48) or (49) as an active ingredient.
(53) A method for screening a diagnostic reagent or a therapeutic agent for a disease caused by a cell proliferation abnormality, wherein promotion or suppression of the expression or function of the nucleic acid described in (48) or (49) serves as an index.
(54) A diagnostic reagent or a therapeutic agent for a disease caused by a cell proliferation abnormality, comprising a substance that suppresses or promotes the expression of a target gene of the nucleic acid described in (48) or (49) as an active ingredient.
(55) A method for screening a diagnostic reagent or a therapeutic agent for a disease caused by a cell proliferation abnormality, wherein suppression or promotion of the expression of a target gene of the nucleic acid described in (48) or (49) serves as an index.
(56) The diagnostic reagent, the therapeutic agent or the screening method described in any one of (48) to (55), wherein the disease caused by a cell proliferation abnormality is a disease selected from the group consisting of cancers, arteriosclerosis, rheumatoid arthritis, prostatic hyperplasia, blood vessel restenosis after percutaneous transvascular coronary angioplasty, fibroid lung, glomerulonephritis and autoimmune diseases.
(57) A cell proliferation suppressant comprising the nucleic acid described in any one of (1) to (4) wherein the SEQ ID NO: is any one of 1, 3, 8, 20, 21, 22, 32 and 36, as an active ingredient.
(58) A cell proliferation suppressant comprising the nucleic acid described in any one of (5) to (7) wherein the SEQ ID NO: is any one of 1337, 1339, 1352, 1371, 1372, 1373, 1386 and 1390, as an active ingredient.
(59) A cell proliferation promoter comprising a nucleic acid consisting of a nucleotide sequence complementary to the nucleic acid described in (57) or (58) as an active ingredient.
(60) A cell proliferation suppressant or proliferation promoter comprising a double-stranded nucleic acid consisting of the nucleic acid described in (57) or (58) and a nucleic acid consisting of a nucleotide sequence complementary to the nucleotide sequence of the nucleic acid as an active ingredient.
(61) A cell proliferation suppressant or proliferation promoter comprising a substance that promotes or suppresses the expression or function of the nucleic acid described in (57) or (58) as an active ingredient.
(62) A method for screening a cell proliferation suppressant or proliferation promoter, wherein promotion or suppression of the expression or function of the nucleic acid described in (57) or (58) serves as an index.
(63) A cell proliferation suppressant or proliferation promoter comprising a substance that suppresses or promotes the expression of a target gene of the nucleic acid described in (57) or (58) as an active ingredient.
(64) A method for screening a cell proliferation suppressant or proliferation promoter, wherein suppression or promotion of the expression of a target gene by the nucleic acid described in (57) or (58) serves as an index.

### Effect of the Invention

According to the present invention, it is possible to provide a novel nucleic acid, a vector that expresses the nucleic acid, a method of detecting the expression and mutation of the nucleic acid, a screening method for a substance that controls the nucleic acid, a method of separating a cell that expresses the nucleic acid, a method of controlling the expression of a target gene of the nucleic acid, a diagnostic reagent or pharmaceutical comprising the nucleic acid or a substance that controls the nucleic acid as an active ingredient, and a diagnostic reagent or pharmaceutical comprising a substance that controls the expression of a target gene of the nucleic acid, as well as a diagnostic reagent or a therapeutic agent for a disease caused by an abnormality of mast cells or mesenchymal stem cells and the like, and diseases such as cancers, an agent that controls cell differentiation or proliferation, and the like.

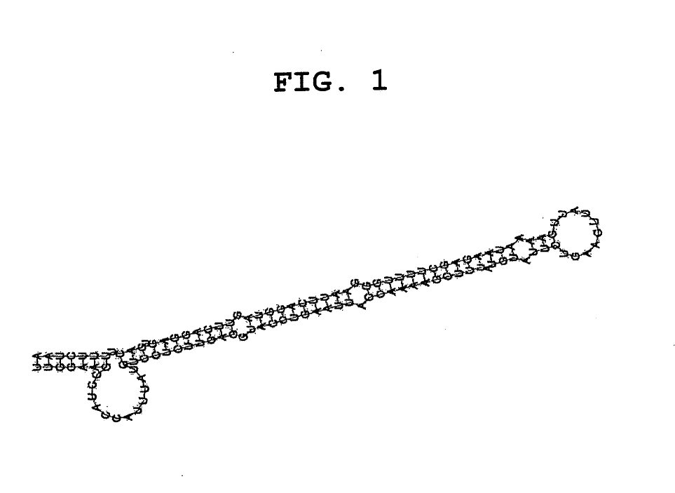

### BRIEF DESCRIPTION OF THE DRAWINGS

[FIG. 1] shows the secondary structure of the nucleotide sequence of KHK_miR_{—}1194 of SEQ ID NO:1580.

### BEST MODE FOR CARRYING OUT THE INVENTION

As the nucleic acid in the present invention, the following nucleic acids can be mentioned. The nucleic acid is preferably a micro-RNA or a derivative thereof, a micro-RNA precursor or a derivative thereof, or a double-stranded nucleic acid (hereinafter also referred to as a nucleic acid of the present invention).
(1) A nucleic acid consisting of the nucleotide sequence of any one of SEQ ID NOs:1 to 1336.
(2) A nucleic acid consisting of a nucleotide sequence having an identity of 90% or more to the nucleotide sequence of any one of SEQ ID NOs:1 to 1336.
(3) A nucleic acid that hybridizes under stringent conditions with a strand complementary to a nucleic acid consisting of the nucleotide sequence of any one of SEQ ID NOs:1 to 1336.
(4) A nucleic acid comprising the nucleic acid described in any one of (1) to (3).
(5) A nucleic acid consisting of the nucleotide sequence of any one of SEQ ID NOs:1337 to 2851.
(6) A nucleic acid consisting of a nucleotide sequence having an identity of 80% or more to the nucleotide sequence of any one of SEQ ID NOs:1337 to 2851.
(7) A nucleic acid that hybridizes under stringent conditions with a strand complementary to a nucleic acid consisting of the nucleotide sequence of any one of SEQ ID NOs:1337 to 2851.
(8) A nucleic acid consisting of a nucleotide sequence complementary to the nucleic acid described in any one of (1) to (7).
(9) A double-stranded nucleic acid consisting of the nucleic acid described in any one of (1) to (7) and a nucleic acid consisting of a nucleotide sequence complementary to the nucleotide sequence of the nucleic acid.

In the present invention, a micro-RNA refers to an RNA that is a cell-derived single-stranded RNA, that has a sequence wherein the surrounding genome sequence, including the sequence, is capable of forming a hairpin structure, and that is capable of being cleaved out from either one chain of the hairpin. The length of the micro-RNA is preferably 15 to 28 nucleotides, more preferably 16 to 28 nucleotides, still more preferably 16 to 26 nucleotides, and particularly preferably 16 to 24 nucleotides. A micro-RNA complementarily binds to an mRNA being a target thereof, to degrade the mRNA or to suppress the translation of the mRNA, and to make post-transcriptional control of gene expression. As a micro-RNA of the present invention, a nucleic acid consisting of the nucleotide sequence of any one of SEQ ID NOs:1 to 1336, and a nucleic acid consisting of a nucleotide sequence having an identity of 90% or more, preferably 95% or more, to the nucleotide sequence of any one of SEQ ID NOs:1 to 1336 can be mentioned. A nucleic acid that hybridizes under stringent conditions with a strand complementary to a nucleic acid consisting of the nucleotide sequence of any one of SEQ ID NOs:1 to 1336 can also be mentioned. As another micro-RNA of the present invention, a nucleic acid comprising these nucleic acids can be mentioned.

In the present invention, a micro-RNA precursor is a nucleic acid, including a micro-RNA, that is about 50 to about 200 nucleotides, preferably about 70 to about 100 nucleotides, long, and that is capable of forming a hairpin structure. A micro-RNA is produced from a micro-RNA precursor via processing by a protein called Dicer. As micro-RNA precursors of the present invention, a nucleic acid consisting of the nucleotide sequence of any one of SEQ ID NOs:1337 to 2851, a nucleic acid consisting of a nucleotide sequence having an identity of 80% or more, preferably 90% or more, and still more preferably 95% or more, to the nucleotide sequence of any one of SEQ ID NOs:1337 to 2851, and a nucleic acid that hybridizes under stringent conditions with a strand complementary to a nucleic acid consisting of the nucleotide sequence of any one of SEQ ID NOs:1337 to 2851 can be mentioned. Because a micro-RNA precursor comprises a sequence of a micro-RNA, and it exhibits a function as a precursor if the micro-RNA is produced from the micro-RNA precursor via processing, any nucleic acid having a homology of 80% or more to the nucleotide sequence of any one of SEQ ID NOs:1337 to 2851 is thought to exhibit a function as a micro-RNA precursor.

Shown in Tables 1-1 to 1-40 are relationships between the nucleotide sequences of SEQ ID NOs:1 to 1336, specifically mentioned as micro-RNAs, and the nucleotide sequences of micro-RNA precursors mentioned as precursors thereof.

TABLE 1-1

| nucleic acid | | micro-RNA |
|---|---|---|
| name | micro-RNA | precursor |
| KHK_miR_1001 | SEQ ID NO: 1 | SEQ ID NO: 1337 |
| KHK_miR_1002 | SEQ ID NO: 2 | SEQ ID NO: 1338 |
| KHK_miR_1003 | SEQ ID NO: 3 | SEQ ID NO: 1339 |
| KHK_miR_1004 | SEQ ID NO: 4 | SEQ ID NO: 1340 |
| KHK_miR_1005 | SEQ ID NO: 5 | SEQ ID NO: 1341 |
| KHK_miR_1006 | SEQ ID NO: 6 | SEQ ID NO: 1342 |
| KHK_miR_1007 | SEQ ID NO: 7 | SEQ ID NO: 1343 |
| | | SEQ ID NO: 1344 |
| | | SEQ ID NO: 1345 |
| | | SEQ ID NO: 1346 |
| | | SEQ ID NO: 1347 |
| | | SEQ ID NO: 1348 |
| | | SEQ ID NO: 1349 |
| | | SEQ ID NO: 1350 |
| | | SEQ ID NO: 1351 |
| KHK_miR_1008 | SEQ ID NO: 8 | SEQ ID NO: 1352 |
| KHK_miR_1009 | SEQ ID NO: 9 | SEQ ID NO: 1353 |
| | | SEQ ID NO: 1354 |
| KHK_miR_1010 | SEQ ID NO: 10 | SEQ ID NO: 1355 |
| KHK_miR_1011 | SEQ ID NO: 11 | SEQ ID NO: 1356 |
| | | SEQ ID NO: 1357 |
| | | SEQ ID NO: 1358 |
| | | SEQ ID NO: 1359 |
| | | SEQ ID NO: 1360 |
| KHK_miR_1012 | SEQ ID NO: 12 | SEQ ID NO: 1361 |
| KHK_miR_1013 | SEQ ID NO: 13 | SEQ ID NO: 1362 |
| KHK_miR_1014 | SEQ ID NO: 14 | SEQ ID NO: 1363 |
| KHK_miR_1015 | SEQ ID NO: 15 | SEQ ID NO: 1364 |
| | | SEQ ID NO: 1365 |
| KHK_miR_1016 | SEQ ID NO: 16 | SEQ ID NO: 1366 |
| KHK_miR_1017 | SEQ ID NO: 17 | SEQ ID NO: 1367 |
| KHK_miR_1018 | SEQ ID NO: 18 | SEQ ID NO: 1368 |
| KHK_miR_1019 | SEQ ID NO: 19 | SEQ ID NO: 1369 |
| | | SEQ ID NO: 1370 |
| KHK_miR_1020 | SEQ ID NO: 20 | SEQ ID NO: 1371 |
| KHK_miR_1021 | SEQ ID NO: 21 | SEQ ID NO: 1372 |
| KHK_miR_1022 | SEQ ID NO: 22 | SEQ ID NO: 1373 |
| KHK_miR_1023 | SEQ ID NO: 23 | SEQ ID NO: 1374 |
| KHK_miR_1024 | SEQ ID NO: 24 | SEQ ID NO: 1375 |

TABLE 1-2

| nucleic acid | | micro-RNA |
|---|---|---|
| name | micro-RNA | precursor |
| | | SEQ ID NO: 1376 |
| KHK_miR_1025 | SEQ ID NO: 25 | SEQ ID NO: 1377 |
| KHK_miR_1026 | SEQ ID NO: 26 | SEQ ID NO: 1378 |
| KHK_miR_1027 | SEQ ID NO: 27 | SEQ ID NO: 1379 |
| | | SEQ ID NO: 1380 |
| | | SEQ ID NO: 1381 |
| KHK_miR_1028 | SEQ ID NO: 28 | SEQ ID NO: 1382 |
| KHK_miR_1029 | SEQ ID NO: 29 | SEQ ID NO: 1383 |
| KHK_miR_1030 | SEQ ID NO: 30 | SEQ ID NO: 1384 |
| KHK_miR_1031 | SEQ ID NO: 31 | SEQ ID NO: 1385 |
| KHK_miR_1032 | SEQ ID NO: 32 | SEQ ID NO: 1386 |
| KHK_miR_1033 | SEQ ID NO: 33 | SEQ ID NO: 1387 |
| KHK_miR_1034 | SEQ ID NO: 34 | SEQ ID NO: 1388 |
| KHK_miR_1035 | SEQ ID NO: 35 | SEQ ID NO: 1389 |
| KHK_miR_1036 | SEQ ID NO: 36 | SEQ ID NO: 1390 |
| KHK_miR_1037 | SEQ ID NO: 37 | SEQ ID NO: 1391 |
| KHK_miR_1038 | SEQ ID NO: 38 | SEQ ID NO: 1392 |
| KHK_miR_1039 | SEQ ID NO: 39 | SEQ ID NO: 1393 |
| KHK_miR_1040 | SEQ ID NO: 40 | SEQ ID NO: 1394 |
| KHK_miR_1041 | SEQ ID NO: 41 | SEQ ID NO: 1395 |
| KHK_miR_1042 | SEQ ID NO: 42 | SEQ ID NO: 1396 |
| KHK_miR_1043 | SEQ ID NO: 43 | SEQ ID NO: 1397 |
| KHK_miR_1044 | SEQ ID NO: 44 | SEQ ID NO: 1398 |
| KHK_miR_1045 | SEQ ID NO: 45 | SEQ ID NO: 1399 |
| KHK_miR_1046 | SEQ ID NO: 46 | SEQ ID NO: 1400 |
| KHK_miR_1047 | SEQ ID NO: 47 | SEQ ID NO: 1401 |
| KHK_miR_1048 | SEQ ID NO: 48 | SEQ ID NO: 1402 |
| | | SEQ ID NO: 1403 |
| KHK_miR_1049 | SEQ ID NO: 49 | SEQ ID NO: 1404 |
| | | SEQ ID NO: 1405 |
| KHK_miR_1050 | SEQ ID NO: 50 | SEQ ID NO: 1406 |
| KHK_miR_1051 | SEQ ID NO: 51 | SEQ ID NO: 1407 |
| KHK_miR_1052 | SEQ ID NO: 52 | SEQ ID NO: 1408 |
| KHK_miR_1053 | SEQ ID NO: 53 | SEQ ID NO: 1409 |
| KHK_miR_1054 | SEQ ID NO: 54 | SEQ ID NO: 1410 |
| KHK_miR_1055 | SEQ ID NO: 55 | SEQ ID NO: 1411 |
| KHK_miR_1056 | SEQ ID NO: 56 | SEQ ID NO: 1412 |
| KHK_miR_1057 | SEQ ID NO: 57 | SEQ ID NO: 1413 |
| KHK_miR_1058 | SEQ ID NO: 58 | SEQ ID NO: 1414 |

TABLE 1-3

| nucleic acid | | micro-RNA |
|---|---|---|
| name | micro-RNA | precursor |
| | | SEQ ID NO: 1415 |
| | | SEQ ID NO: 1416 |
| | | SEQ ID NO: 1417 |
| | | SEQ ID NO: 1418 |
| KHK_miR_1059 | SEQ ID NO: 59 | SEQ ID NO: 1419 |
| KHK_miR_1060 | SEQ ID NO: 60 | SEQ ID NO: 1420 |
| KHK_miR_1061 | SEQ ID NO: 61 | SEQ ID NO: 1421 |
| KHK_miR_1062 | SEQ ID NO: 62 | SEQ ID NO: 1422 |
| KHK_miR_1063 | SEQ ID NO: 63 | SEQ ID NO: 1423 |
| KHK_miR_1064 | SEQ ID NO: 64 | SEQ ID NO: 1424 |
| KHK_miR_1065 | SEQ ID NO: 65 | SEQ ID NO: 1425 |
| KHK_miR_1066 | SEQ ID NO: 66 | SEQ ID NO: 1426 |
| KHK_miR_1067 | SEQ ID NO: 67 | SEQ ID NO: 1427 |
| KHK_miR_1068 | SEQ ID NO: 68 | SEQ ID NO: 1428 |
| KHK_miR_1069 | SEQ ID NO: 69 | SEQ ID NO: 1429 |
| KHK_miR_1070 | SEQ ID NO: 70 | SEQ ID NO: 1430 |
| KHK_miR_1071 | SEQ ID NO: 71 | SEQ ID NO: 1431 |
| KHK_miR_1072 | SEQ ID NO: 72 | SEQ ID NO: 1432 |
| KHK_miR_1073 | SEQ ID NO: 73 | SEQ ID NO: 1433 |
| KHK_miR_1074 | SEQ ID NO: 74 | SEQ ID NO: 1434 |
| KHK_miR_1075 | SEQ ID NO: 75 | SEQ ID NO: 1435 |
| | | SEQ ID NO: 1436 |
| KHK_miR_1076 | SEQ ID NO: 76 | SEQ ID NO: 1437 |
| KHK_miR_1077 | SEQ ID NO: 77 | SEQ ID NO: 1438 |
| KHK_miR_1078 | SEQ ID NO: 78 | SEQ ID NO: 1439 |
| KHK_miR_1079 | SEQ ID NO: 79 | SEQ ID NO: 1440 |
| | | SEQ ID NO: 1441 |
| | | SEQ ID NO: 1442 |
| KHK_miR_1080 | SEQ ID NO: 80 | SEQ ID NO: 1443 |
| KHK_miR_1081 | SEQ ID NO: 81 | SEQ ID NO: 1444 |
| KHK_miR_1082 | SEQ ID NO: 82 | SEQ ID NO: 1445 |
| KHK_miR_1083 | SEQ ID NO: 83 | SEQ ID NO: 1446 |
| KHK_miR_1084 | SEQ ID NO: 84 | SEQ ID NO: 1447 |
| KHK_miR_1085 | SEQ ID NO: 85 | SEQ ID NO: 1448 |
| KHK_miR_1086 | SEQ ID NO: 86 | SEQ ID NO: 1449 |
| KHK_miR_1087 | SEQ ID NO: 87 | SEQ ID NO: 1450 |
| KHK_miR_1088 | SEQ ID NO: 88 | SEQ ID NO: 1451 |
| KHK_miR_1089 | SEQ ID NO: 89 | SEQ ID NO: 1452 |
| KHK_miR_1090 | SEQ ID NO: 90 | SEQ ID NO: 1453 |

TABLE 1-4

| nucleic acid | | micro-RNA |
|---|---|---|
| name | micro-RNA | precursor |
| KHK_miR_1091 | SEQ ID NO: 91 | SEQ ID NO: 1454 |
| KHK_miR_1092 | SEQ ID NO: 92 | SEQ ID NO: 1455 |
| KHK_miR_1093 | SEQ ID NO: 93 | SEQ ID NO: 1456 |
| | | SEQ ID NO: 1457 |
| | | SEQ ID NO: 1458 |
| | | SEQ ID NO: 1459 |
| KHK_miR_1094 | SEQ ID NO: 94 | SEQ ID NO: 1460 |
| KHK_miR_1095 | SEQ ID NO: 95 | SEQ ID NO: 1461 |
| KHK_miR_1096 | SEQ ID NO: 96 | SEQ ID NO: 1462 |
| KHK_miR_1097 | SEQ ID NO: 97 | SEQ ID NO: 1463 |
| KHK_miR_1098 | SEQ ID NO: 98 | SEQ ID NO: 1464 |
| KHK_miR_1099 | SEQ ID NO: 99 | SEQ ID NO: 1465 |
| KHK_miR_1100 | SEQ ID NO: 100 | SEQ ID NO: 1466 |
| KHK_miR_1101 | SEQ ID NO: 101 | SEQ ID NO: 1467 |
| KHK_miR_1102 | SEQ ID NO: 102 | SEQ ID NO: 1468 |
| KHK_miR_1103 | SEQ ID NO: 103 | SEQ ID NO: 1469 |
| KHK_miR_1104 | SEQ ID NO: 104 | SEQ ID NO: 1470 |
| | | SEQ ID NO: 1471 |
| KHK_miR_1105 | SEQ ID NO: 105 | SEQ ID NO: 1472 |
| KHK_miR_1106 | SEQ ID NO: 106 | SEQ ID NO: 1473 |
| KHK_miR_1107 | SEQ ID NO: 107 | SEQ ID NO: 1474 |
| KHK_miR_1108 | SEQ ID NO: 108 | SEQ ID NO: 1475 |
| KHK_miR_1109 | SEQ ID NO: 109 | SEQ ID NO: 1476 |
| KHK_miR_1110 | SEQ ID NO: 110 | SEQ ID NO: 1477 |
| KHK_miR_1111 | SEQ ID NO: 111 | SEQ ID NO: 1478 |
| KHK_miR_1112 | SEQ ID NO: 112 | SEQ ID NO: 1479 |
| | | SEQ ID NO: 1480 |
| KHK_miR_1113 | SEQ ID NO: 113 | SEQ ID NO: 1481 |
| KHK_miR_1114 | SEQ ID NO: 114 | SEQ ID NO: 1482 |
| KHK_miR_1115 | SEQ ID NO: 115 | SEQ ID NO: 1483 |
| KHK_miR_1116 | SEQ ID NO: 116 | SEQ ID NO: 1484 |
| KHK_miR_1117 | SEQ ID NO: 117 | SEQ ID NO: 1485 |
| KHK_miR_1118 | SEQ ID NO: 118 | SEQ ID NO: 1486 |
| KHK_miR_1119 | SEQ ID NO: 119 | SEQ ID NO: 1487 |
| KHK_miR_1120 | SEQ ID NO: 120 | SEQ ID NO: 1488 |
| KHK_miR_1121 | SEQ ID NO: 121 | SEQ ID NO: 1489 |
| KHK_miR_1122 | SEQ ID NO: 122 | SEQ ID NO: 1490 |
| KHK_miR_1123 | SEQ ID NO: 123 | SEQ ID NO: 1491 |
| KHK_miR_1124 | SEQ ID NO: 124 | SEQ ID NO: 1492 |

TABLE 1-5

| nucleic acid | | micro-RNA |
|---|---|---|
| name | micro-RNA | precursor |
| KHK_miR_1125 | SEQ ID NO: 125 | SEQ ID NO: 1493 |
| KHK_miR_1126 | SEQ ID NO: 126 | SEQ ID NO: 1494 |
| | | SEQ ID NO: 1495 |
| | | SEQ ID NO: 1496 |
| KHK_miR_1127 | SEQ ID NO: 127 | SEQ ID NO: 1497 |
| KHK_miR_1128 | SEQ ID NO: 128 | SEQ ID NO: 1498 |
| KHK_miR_1129 | SEQ ID NO: 129 | SEQ ID NO: 1499 |
| KHK_miR_1130 | SEQ ID NO: 130 | SEQ ID NO: 1500 |
| KHK_miR_1131 | SEQ ID NO: 131 | SEQ ID NO: 1501 |
| KHK_miR_1132 | SEQ ID NO: 132 | SEQ ID NO: 1502 |
| KHK_miR_1133 | SEQ ID NO: 133 | SEQ ID NO: 1503 |
| KHK_miR_1134 | SEQ ID NO: 134 | SEQ ID NO: 1504 |
| KHK_miR_1135 | SEQ ID NO: 135 | SEQ ID NO: 1505 |
| KHK_miR_1136 | SEQ ID NO: 136 | SEQ ID NO: 1506 |
| KHK_miR_1137 | SEQ ID NO: 137 | SEQ ID NO: 1507 |
| KHK_miR_1138 | SEQ ID NO: 138 | SEQ ID NO: 1508 |
| KHK_miR_1139 | SEQ ID NO: 139 | SEQ ID NO: 1509 |
| KHK_miR_1140 | SEQ ID NO: 140 | SEQ ID NO: 1510 |
| KHK_miR_1141 | SEQ ID NO: 141 | SEQ ID NO: 1511 |
| KHK_miR_1142 | SEQ ID NO: 142 | SEQ ID NO: 1512 |
| KHK_miR_1143 | SEQ ID NO: 143 | SEQ ID NO: 1513 |
| KHK_miR_1144 | SEQ ID NO: 144 | SEQ ID NO: 1514 |
| KHK_miR_1145 | SEQ ID NO: 145 | SEQ ID NO: 1515 |
| KHK_miR_1146 | SEQ ID NO: 146 | SEQ ID NO: 1516 |
| KHK_miR_1147 | SEQ ID NO: 147 | SEQ ID NO: 1517 |
| KHK_miR_1148 | SEQ ID NO: 148 | SEQ ID NO: 1518 |
| KHK_miR_1149 | SEQ ID NO: 149 | SEQ ID NO: 1519 |
| KHK_miR_1150 | SEQ ID NO: 150 | SEQ ID NO: 1520 |
| KHK_miR_1151 | SEQ ID NO: 151 | SEQ ID NO: 1521 |
| KHK_miR_1152 | SEQ ID NO: 152 | SEQ ID NO: 1522 |
| | | SEQ ID NO: 1523 |
| | | SEQ ID NO: 1524 |
| KHK_miR_1153 | SEQ ID NO: 153 | SEQ ID NO: 1525 |
| KHK_miR_1154 | SEQ ID NO: 154 | SEQ ID NO: 1526 |
| KHK_miR_1155 | SEQ ID NO: 155 | SEQ ID NO: 1527 |
| KHK_miR_1156 | SEQ ID NO: 156 | SEQ ID NO: 1528 |
| KHK_miR_1157 | SEQ ID NO: 157 | SEQ ID NO: 1529 |
| KHK_miR_1158 | SEQ ID NO: 158 | SEQ ID NO: 1530 |
| KHK_miR_1159 | SEQ ID NO: 159 | SEQ ID NO: 1531 |

TABLE 1-6

| nucleic acid | | micro-RNA |
|---|---|---|
| name | micro-RNA | precursor |
| KHK_miR_1160 | SEQ ID NO: 160 | SEQ ID NO: 1532 |
| KHK_miR_1161 | SEQ ID NO: 161 | SEQ ID NO: 1533 |
| KHK_miR_1162 | SEQ ID NO: 162 | SEQ ID NO: 1534 |
| KHK_miR_1163 | SEQ ID NO: 163 | SEQ ID NO: 1535 |
| KHK_miR_1164 | SEQ ID NO: 164 | SEQ ID NO: 1536 |
| KHK_miR_1165 | SEQ ID NO: 165 | SEQ ID NO: 1537 |
| KHK_miR_1166 | SEQ ID NO: 166 | SEQ ID NO: 1538 |
| KHK_miR_1167 | SEQ ID NO: 167 | SEQ ID NO: 1539 |
| KHK_miR_1168 | SEQ ID NO: 168 | SEQ ID NO: 1540 |
| KHK_miR_1169 | SEQ ID NO: 169 | SEQ ID NO: 1541 |
| KHK_miR_1170 | SEQ ID NO: 170 | SEQ ID NO: 1542 |
| KHK_miR_1171 | SEQ ID NO: 171 | SEQ ID NO: 1543 |
| KHK_miR_1172 | SEQ ID NO: 172 | SEQ ID NO: 1544 |
| KHK_miR_1173 | SEQ ID NO: 173 | SEQ ID NO: 1545 |
| KHK_miR_1174 | SEQ ID NO: 174 | SEQ ID NO: 1546 |
| KHK_miR_1175 | SEQ ID NO: 175 | SEQ ID NO: 1547 |
| KHK_miR_1176 | SEQ ID NO: 176 | SEQ ID NO: 1548 |
| KHK_miR_1177 | SEQ ID NO: 177 | SEQ ID NO: 1549 |
| KHK_miR_1178 | SEQ ID NO: 178 | SEQ ID NO: 1550 |
| | | SEQ ID NO: 1551 |
| | | SEQ ID NO: 1552 |
| | | SEQ ID NO: 1553 |
| | | SEQ ID NO: 1554 |
| | | SEQ ID NO: 1555 |
| KHK_miR_1179 | SEQ ID NO: 179 | SEQ ID NO: 1556 |
| KHK_miR_1180 | SEQ ID NO: 180 | SEQ ID NO: 1557 |
| KHK_miR_1181 | SEQ ID NO: 181 | SEQ ID NO: 1558 |
| | | SEQ ID NO: 1559 |
| KHK_miR_1182 | SEQ ID NO: 182 | SEQ ID NO: 1560 |
| KHK_miR_1183 | SEQ ID NO: 183 | SEQ ID NO: 1561 |
| KHK_miR_1184 | SEQ ID NO: 184 | SEQ ID NO: 1562 |
| KHK_miR_1185 | SEQ ID NO: 185 | SEQ ID NO: 1563 |
| KHK_miR_1186 | SEQ ID NO: 186 | SEQ ID NO: 1564 |
| KHK_miR_1187 | SEQ ID NO: 187 | SEQ ID NO: 1565 |
| KHK_miR_1188 | SEQ ID NO: 188 | SEQ ID NO: 1566 |
| KHK_miR_1189 | SEQ ID NO: 189 | SEQ ID NO: 1567 |
| KHK_miR_1190 | SEQ ID NO: 190 | SEQ ID NO: 1568 |
| KHK_miR_1191 | SEQ ID NO: 191 | SEQ ID NO: 1569 |
| KHK_miR_1192 | SEQ ID NO: 192 | SEQ ID NO: 1570 |

TABLE 1-7

| nucleic acid | | micro-RNA |
|---|---|---|
| name | micro-RNA | precursor |
| KHK_miR_1193 | SEQ ID NO: 193 | SEQ ID NO: 1571 |
| KHK_miR_1194 | SEQ ID NO: 194 | SEQ ID NO: 1572 |
| KHK_miR_1195 | SEQ ID NO: 195 | SEQ ID NO: 1573 |
| KHK_miR_1196 | SEQ ID NO: 196 | SEQ ID NO: 1574 |
| KHK_miR_1197 | SEQ ID NO: 197 | SEQ ID NO: 1575 |
| KHK_miR_1198 | SEQ ID NO: 198 | SEQ ID NO: 1576 |
| KHK_miR_1199 | SEQ ID NO: 199 | SEQ ID NO: 1577 |
| KHK_miR_1200 | SEQ ID NO: 200 | SEQ ID NO: 1578 |
| KHK_miR_1201 | SEQ ID NO: 201 | SEQ ID NO: 1579 |
| KHK_miR_1202 | SEQ ID NO: 202 | SEQ ID NO: 1580 |
| KHK_miR_1203 | SEQ ID NO: 203 | SEQ ID NO: 1581 |
| KHK_miR_1204 | SEQ ID NO: 204 | SEQ ID NO: 1582 |
| KHK_miR_1205 | SEQ ID NO: 205 | SEQ ID NO: 1583 |
| KHK_miR_1206 | SEQ ID NO: 206 | SEQ ID NO: 1584 |
| KHK_miR_1207 | SEQ ID NO: 207 | SEQ ID NO: 1585 |
| KHK_miR_1208 | SEQ ID NO: 208 | SEQ ID NO: 1586 |
| | | SEQ ID NO: 1587 |
| KHK_miR_1209 | SEQ ID NO: 209 | SEQ ID NO: 1588 |
| KHK_miR_1210 | SEQ ID NO: 210 | SEQ ID NO: 1589 |
| KHK_miR_1211 | SEQ ID NO: 211 | SEQ ID NO: 1590 |
| KHK_miR_1212 | SEQ ID NO: 212 | SEQ ID NO: 1591 |
| KHK_miR_1213 | SEQ ID NO: 213 | SEQ ID NO: 1592 |
| | | SEQ ID NO: 1593 |
| | | SEQ ID NO: 1594 |
| KHK_miR_1214 | SEQ ID NO: 214 | SEQ ID NO: 1595 |
| | | SEQ ID NO: 1596 |
| KHK_miR_1215 | SEQ ID NO: 215 | SEQ ID NO: 1597 |
| KHK_miR_1216 | SEQ ID NO: 216 | SEQ ID NO: 1598 |
| KHK_miR_1217 | SEQ ID NO: 217 | SEQ ID NO: 1599 |
| KHK_miR_1218 | SEQ ID NO: 218 | SEQ ID NO: 1600 |
| KHK_miR_1219 | SEQ ID NO: 219 | SEQ ID NO: 1601 |
| KHK_miR_1220 | SEQ ID NO: 220 | SEQ ID NO: 1602 |
| KHK_miR_1221 | SEQ ID NO: 221 | SEQ ID NO: 1603 |
| KHK_miR_1222 | SEQ ID NO: 222 | SEQ ID NO: 1604 |
| KHK_miR_1223 | SEQ ID NO: 223 | SEQ ID NO: 1605 |
| KHK_miR_1224 | SEQ ID NO: 224 | SEQ ID NO: 1606 |
| KHK_miR_1225 | SEQ ID NO: 225 | SEQ ID NO: 1607 |
| KHK_miR_1226 | SEQ ID NO: 226 | SEQ ID NO: 1608 |
| KHK_miR_1227 | SEQ ID NO: 227 | SEQ ID NO: 1609 |

TABLE 1-8

| nucleic acid | | micro-RNA |
|---|---|---|
| name | micro-RNA | precursor |
| KHK_miR_1228 | SEQ ID NO: 228 | SEQ ID NO: 1610 |
| KHK_miR_1229 | SEQ ID NO: 229 | SEQ ID NO: 1611 |
| KHK_miR_1230 | SEQ ID NO: 230 | SEQ ID NO: 1612 |
| KHK_miR_1231 | SEQ ID NO: 231 | SEQ ID NO: 1613 |
| KHK_miR_1232 | SEQ ID NO: 232 | SEQ ID NO: 1614 |
| KHK_miR_1233 | SEQ ID NO: 233 | SEQ ID NO: 1615 |
| | | SEQ ID NO: 1616 |
| | | SEQ ID NO: 1617 |
| | | SEQ ID NO: 1618 |
| KHK_miR_1234 | SEQ ID NO: 234 | SEQ ID NO: 1619 |
| KHK_miR_1235 | SEQ ID NO: 235 | SEQ ID NO: 1620 |
| KHK_miR_1236 | SEQ ID NO: 236 | SEQ ID NO: 1621 |
| KHK_miR_1237 | SEQ ID NO: 237 | SEQ ID NO: 1622 |
| KHK_miR_1238 | SEQ ID NO: 238 | SEQ ID NO: 1623 |
| KHK_miR_1239 | SEQ ID NO: 239 | SEQ ID NO: 1624 |
| KHK_miR_1240 | SEQ ID NO: 240 | SEQ ID NO: 1625 |
| KHK_miR_1241 | SEQ ID NO: 241 | SEQ ID NO: 1626 |
| | | SEQ ID NO: 1627 |
| KHK_miR_1242 | SEQ ID NO: 242 | SEQ ID NO: 1628 |
| KHK_miR_1243 | SEQ ID NO: 243 | SEQ ID NO: 1629 |
| KHK_miR_1244 | SEQ ID NO: 244 | SEQ ID NO: 1630 |
| KHK_miR_1245 | SEQ ID NO: 245 | SEQ ID NO: 1631 |
| KHK_miR_1246 | SEQ ID NO: 246 | SEQ ID NO: 1632 |
| KHK_miR_1247 | SEQ ID NO: 247 | SEQ ID NO: 1633 |
| KHK_miR_1248 | SEQ ID NO: 248 | SEQ ID NO: 1634 |
| KHK_miR_1249 | SEQ ID NO: 249 | SEQ ID NO: 1635 |
| KHK_miR_1250 | SEQ ID NO: 250 | SEQ ID NO: 1636 |
| KHK_miR_1251 | SEQ ID NO: 251 | SEQ ID NO: 1637 |
| KHK_miR_1252 | SEQ ID NO: 252 | SEQ ID NO: 1638 |
| | | SEQ ID NO: 1639 |
| KHK_miR_1253 | SEQ ID NO: 253 | SEQ ID NO: 1640 |
| KHK_miR_1254 | SEQ ID NO: 254 | SEQ ID NO: 1641 |
| KHK_miR_1255 | SEQ ID NO: 255 | SEQ ID NO: 1642 |
| | | SEQ ID NO: 1643 |
| | | SEQ ID NO: 1644 |
| KHK_miR_1256 | SEQ ID NO: 256 | SEQ ID NO: 1645 |
| KHK_miR_1257 | SEQ ID NO: 257 | SEQ ID NO: 1646 |
| KHK_miR_1258 | SEQ ID NO: 258 | SEQ ID NO: 1647 |
| | | SEQ ID NO: 1648 |

TABLE 1-9

| nucleic acid | | micro-RNA |
|---|---|---|
| name | micro-RNA | precursor |
| KHK_miR_1259 | SEQ ID NO: 259 | SEQ ID NO: 1649 |
| KHK_miR_1260 | SEQ ID NO: 260 | SEQ ID NO: 1650 |
| KHK_miR_1261 | SEQ ID NO: 261 | SEQ ID NO: 1651 |
| | | SEQ ID NO: 1652 |
| KHK_miR_1262 | SEQ ID NO: 262 | SEQ ID NO: 1653 |
| KHK_miR_1263 | SEQ ID NO: 263 | SEQ ID NO: 1654 |
| KHK_miR_1264 | SEQ ID NO: 264 | SEQ ID NO: 1655 |
| KHK_miR_1265 | SEQ ID NO: 265 | SEQ ID NO: 1656 |
| KHK_miR_1266 | SEQ ID NO: 266 | SEQ ID NO: 1657 |
| KHK_miR_1267 | SEQ ID NO: 267 | SEQ ID NO: 1658 |
| KHK_miR_1268 | SEQ ID NO: 268 | SEQ ID NO: 1659 |
| KHK_miR_1269 | SEQ ID NO: 269 | SEQ ID NO: 1660 |
| KHK_miR_1270 | SEQ ID NO: 270 | SEQ ID NO: 1661 |
| KHK_miR_1271 | SEQ ID NO: 271 | SEQ ID NO: 1662 |
| KHK_miR_1272 | SEQ ID NO: 272 | SEQ ID NO: 1663 |
| KHK_miR_1273 | SEQ ID NO: 273 | SEQ ID NO: 1664 |
| KHK_miR_1274 | SEQ ID NO: 274 | SEQ ID NO: 1665 |
| KHK_miR_1275 | SEQ ID NO: 275 | SEQ ID NO: 1666 |
| KHK_miR_1276 | SEQ ID NO: 276 | SEQ ID NO: 1667 |
| KHK_miR_1277 | SEQ ID NO: 277 | SEQ ID NO: 1668 |
| KHK_miR_1278 | SEQ ID NO: 278 | SEQ ID NO: 1669 |
| KHK_miR_1279 | SEQ ID NO: 279 | SEQ ID NO: 1670 |
| KHK_miR_1280 | SEQ ID NO: 280 | SEQ ID NO: 1671 |
| KHK_miR_1281 | SEQ ID NO: 281 | SEQ ID NO: 1672 |
| | | SEQ ID NO: 1673 |
| | | SEQ ID NO: 1674 |
| | | SEQ ID NO: 1675 |
| KHK_miR_1282 | SEQ ID NO: 282 | SEQ ID NO: 1676 |
| KHK_miR_1283 | SEQ ID NO: 283 | SEQ ID NO: 1677 |
| KHK_miR_1284 | SEQ ID NO: 284 | SEQ ID NO: 1678 |
| KHK_miR_1285 | SEQ ID NO: 285 | SEQ ID NO: 1679 |
| KHK_miR_1286 | SEQ ID NO: 286 | SEQ ID NO: 1680 |
| KHK_miR_1287 | SEQ ID NO: 287 | SEQ ID NO: 1681 |
| KHK_miR_1288 | SEQ ID NO: 288 | SEQ ID NO: 1682 |
| KHK_miR_1289 | SEQ ID NO: 289 | SEQ ID NO: 1683 |
| KHK_miR_1290 | SEQ ID NO: 290 | SEQ ID NO: 1684 |
| KHK_miR_1291 | SEQ ID NO: 291 | SEQ ID NO: 1685 |
| KHK_miR_1292 | SEQ ID NO: 292 | SEQ ID NO: 1686 |
| | | SEQ ID NO: 1687 |

TABLE 1-10

| nucleic acid | | micro-RNA |
|---|---|---|
| name | micro-RNA | precursor |
| KHK_miR_1293 | SEQ ID NO: 293 | SEQ ID NO: 1688 |
| KHK_miR_1294 | SEQ ID NO: 294 | SEQ ID NO: 1689 |
| KHK_miR_1295 | SEQ ID NO: 295 | SEQ ID NO: 1690 |
| KHK_miR_1296 | SEQ ID NO: 296 | SEQ ID NO: 1691 |
| KHK_miR_1297 | SEQ ID NO: 297 | SEQ ID NO: 1692 |
| KHK_miR_1298 | SEQ ID NO: 298 | SEQ ID NO: 1693 |
| KHK_miR_1299 | SEQ ID NO: 299 | SEQ ID NO: 1694 |
| KHK_miR_1300 | SEQ ID NO: 300 | SEQ ID NO: 1695 |
| KHK_miR_1301 | SEQ ID NO: 301 | SEQ ID NO: 1696 |
| KHK_miR_1302 | SEQ ID NO: 302 | SEQ ID NO: 1697 |
| KHK_miR_1303 | SEQ ID NO: 303 | SEQ ID NO: 1698 |
| KHK_miR_1304 | SEQ ID NO: 304 | SEQ ID NO: 1699 |
| KHK_miR_1305 | SEQ ID NO: 305 | SEQ ID NO: 1700 |
| KHK_miR_1306 | SEQ ID NO: 306 | SEQ ID NO: 1701 |
| KHK_miR_1307 | SEQ ID NO: 307 | SEQ ID NO: 1702 |
| KHK_miR_1308 | SEQ ID NO: 308 | SEQ ID NO: 1703 |
| | | SEQ ID NO: 1704 |
| | | SEQ ID NO: 1705 |
| KHK_miR_1309 | SEQ ID NO: 309 | SEQ ID NO: 1706 |
| | | SEQ ID NO: 1707 |
| KHK_miR_1310 | SEQ ID NO: 310 | SEQ ID NO: 1708 |
| KHK_miR_1311 | SEQ ID NO: 311 | SEQ ID NO: 1709 |
| KHK_miR_1312 | SEQ ID NO: 312 | SEQ ID NO: 1710 |
| KHK_miR_1313 | SEQ ID NO: 313 | SEQ ID NO: 1711 |
| KHK_miR_1314 | SEQ ID NO: 314 | SEQ ID NO: 1712 |
| KHK_miR_1315 | SEQ ID NO: 315 | SEQ ID NO: 1713 |
| | | SEQ ID NO: 1714 |
| KHK_miR_1316 | SEQ ID NO: 316 | SEQ ID NO: 1715 |
| KHK_miR_1317 | SEQ ID NO: 317 | SEQ ID NO: 1716 |
| KHK_miR_1318 | SEQ ID NO: 318 | SEQ ID NO: 1717 |
| KHK_miR_1319 | SEQ ID NO: 319 | SEQ ID NO: 1718 |
| KHK_miR_1320 | SEQ ID NO: 320 | SEQ ID NO: 1719 |
| KHK_miR_1321 | SEQ ID NO: 321 | SEQ ID NO: 1720 |
| KHK_miR_1322 | SEQ ID NO: 322 | SEQ ID NO: 1721 |
| KHK_miR_1323 | SEQ ID NO: 323 | SEQ ID NO: 1722 |
| KHK_miR_1324 | SEQ ID NO: 324 | SEQ ID NO: 1723 |
| KHK_miR_1325 | SEQ ID NO: 325 | SEQ ID NO: 1724 |
| KHK_miR_1326 | SEQ ID NO: 326 | SEQ ID NO: 1725 |
| | | SEQ ID NO: 1726 |

TABLE 1-11

| nucleic acid | | micro-RNA |
|---|---|---|
| name | micro-RNA | precursor |
| | | SEQ ID NO: 1727 |
| KHK_miR_1327 | SEQ ID NO: 327 | SEQ ID NO: 1728 |
| KHK_miR_1328 | SEQ ID NO: 328 | SEQ ID NO: 1729 |
| | | SEQ ID NO: 1730 |
| | | SEQ ID NO: 1731 |
| KHK_miR_1329 | SEQ ID NO: 329 | SEQ ID NO: 1732 |
| KHK_miR_1330 | SEQ ID NO: 330 | SEQ ID NO: 1733 |
| KHK_miR_1331 | SEQ ID NO: 331 | SEQ ID NO: 1734 |
| | | SEQ ID NO: 1735 |
| KHK_miR_1332 | SEQ ID NO: 332 | SEQ ID NO: 1736 |
| | | SEQ ID NO: 1737 |
| KHK_miR_1333 | SEQ ID NO: 333 | SEQ ID NO: 1738 |
| KHK_miR_1334 | SEQ ID NO: 334 | SEQ ID NO: 1739 |
| KHK_miR_1335 | SEQ ID NO: 335 | SEQ ID NO: 1740 |
| KHK_miR_1336 | SEQ ID NO: 336 | SEQ ID NO: 1741 |
| KHK_miR_1337 | SEQ ID NO: 337 | SEQ ID NO: 1742 |
| KHK_miR_1338 | SEQ ID NO: 338 | SEQ ID NO: 1743 |
| KHK_miR_1339 | SEQ ID NO: 339 | SEQ ID NO: 1744 |
| KHK_miR_1340 | SEQ ID NO: 340 | SEQ ID NO: 1745 |
| KHK_miR_1341 | SEQ ID NO: 341 | SEQ ID NO: 1746 |
| KHK_miR_1342 | SEQ ID NO: 342 | SEQ ID NO: 1747 |
| KHK_miR_1343 | SEQ ID NO: 343 | SEQ ID NO: 1748 |
| KHK_miR_1344 | SEQ ID NO: 344 | SEQ ID NO: 1749 |
| KHK_miR_1345 | SEQ ID NO: 345 | SEQ ID NO: 1750 |
| KHK_miR_1346 | SEQ ID NO: 346 | SEQ ID NO: 1751 |
| KHK_miR_1347 | SEQ ID NO: 347 | SEQ ID NO: 1752 |
| KHK_miR_1348 | SEQ ID NO: 348 | SEQ ID NO: 1753 |
| KHK_miR_1349 | SEQ ID NO: 349 | SEQ ID NO: 1754 |
| KHK_miR_1350 | SEQ ID NO: 350 | SEQ ID NO: 1755 |
| KHK_miR_1351 | SEQ ID NO: 351 | SEQ ID NO: 1756 |
| KHK_miR_1352 | SEQ ID NO: 352 | SEQ ID NO: 1757 |
| KHK_miR_1353 | SEQ ID NO: 353 | SEQ ID NO: 1758 |
| KHK_miR_1354 | SEQ ID NO: 354 | SEQ ID NO: 1759 |
| KHK_miR_1355 | SEQ ID NO: 355 | SEQ ID NO: 1760 |
| KHK_miR_1356 | SEQ ID NO: 356 | SEQ ID NO: 1761 |
| KHK_miR_1357 | SEQ ID NO: 357 | SEQ ID NO: 1762 |
| KHK_miR_1358 | SEQ ID NO: 358 | SEQ ID NO: 1763 |
| KHK_miR_1359 | SEQ ID NO: 359 | SEQ ID NO: 1764 |
| KHK_miR_1360 | SEQ ID NO: 360 | SEQ ID NO: 1765 |

TABLE 1-12

| nucleic acid | | micro-RNA |
|---|---|---|
| name | micro-RNA | precursor |
| | | SEQ ID NO: 1766 |
| | | SEQ ID NO: 1767 |
| KHK_miR_1361 | SEQ ID NO: 361 | SEQ ID NO: 1768 |
| KHK_miR_1362 | SEQ ID NO: 362 | SEQ ID NO: 1769 |
| KHK_miR_1363 | SEQ ID NO: 363 | SEQ ID NO: 1770 |
| | | SEQ ID NO: 1771 |
| KHK_miR_1364 | SEQ ID NO: 364 | SEQ ID NO: 1772 |
| | | SEQ ID NO: 1773 |
| | | SEQ ID NO: 1774 |
| | | SEQ ID NO: 1775 |
| | | SEQ ID NO: 1776 |
| | | SEQ ID NO: 1777 |
| KHK_miR_1365 | SEQ ID NO: 365 | SEQ ID NO: 1778 |
| KHK_miR_1366 | SEQ ID NO: 366 | SEQ ID NO: 1779 |
| KHK_miR_1367 | SEQ ID NO: 367 | SEQ ID NO: 1780 |
| KHK_miR_1368 | SEQ ID NO: 368 | SEQ ID NO: 1781 |
| KHK_miR_1369 | SEQ ID NO: 369 | SEQ ID NO: 1782 |
| KHK_miR_1370 | SEQ ID NO: 370 | SEQ ID NO: 1783 |
| KHK_miR_1371 | SEQ ID NO: 371 | SEQ ID NO: 1784 |
| KHK_miR_1372 | SEQ ID NO: 372 | SEQ ID NO: 1785 |
| KHK_miR_1373 | SEQ ID NO: 373 | SEQ ID NO: 1786 |
| KHK_miR_1374 | SEQ ID NO: 374 | SEQ ID NO: 1787 |
| KHK_miR_1375 | SEQ ID NO: 375 | SEQ ID NO: 1788 |
| KHK_miR_1376 | SEQ ID NO: 376 | SEQ ID NO: 1789 |
| KHK_miR_1377 | SEQ ID NO: 377 | SEQ ID NO: 1790 |
| KHK_miR_1378 | SEQ ID NO: 378 | SEQ ID NO: 1791 |
| KHK_miR_1379 | SEQ ID NO: 379 | SEQ ID NO: 1792 |
| | | SEQ ID NO: 1793 |
| KHK_miR_1380 | SEQ ID NO: 380 | SEQ ID NO: 1794 |
| KHK_miR_1381 | SEQ ID NO: 381 | SEQ ID NO: 1795 |
| KHK_miR_1382 | SEQ ID NO: 382 | SEQ ID NO: 1796 |
| KHK_miR_1383 | SEQ ID NO: 383 | SEQ ID NO: 1797 |
| KHK_miR_1384 | SEQ ID NO: 384 | SEQ ID NO: 1798 |
| KHK_miR_1385 | SEQ ID NO: 385 | SEQ ID NO: 1799 |
| KHK_miR_1386 | SEQ ID NO: 386 | SEQ ID NO: 1800 |
| KHK_miR_1387 | SEQ ID NO: 387 | SEQ ID NO: 1801 |
| KHK_miR_1388 | SEQ ID NO: 388 | SEQ ID NO: 1802 |
| KHK_miR_1389 | SEQ ID NO: 389 | SEQ ID NO: 1803 |
| KHK_miR_1390 | SEQ ID NO: 390 | SEQ ID NO: 1804 |

TABLE 1-13

| nucleic acid | | micro-RNA |
|---|---|---|
| name | micro-RNA | precursor |
| KHK_miR_1391 | SEQ ID NO: 391 | SEQ ID NO: 1805 |
| KHK_miR_1392 | SEQ ID NO: 392 | SEQ ID NO: 1806 |
| KHK_miR_1393 | SEQ ID NO: 393 | SEQ ID NO: 1807 |
| KHK_miR_1394 | SEQ ID NO: 394 | SEQ ID NO: 1808 |
| KHK_miR_1395 | SEQ ID NO: 395 | SEQ ID NO: 1809 |
| KHK_miR_1396 | SEQ ID NO: 396 | SEQ ID NO: 1810 |
| KHK_miR_1397 | SEQ ID NO: 397 | SEQ ID NO: 1811 |
| KHK_miR_1398 | SEQ ID NO: 398 | SEQ ID NO: 1812 |
| | | SEQ ID NO: 1813 |
| KHK_miR_1399 | SEQ ID NO: 399 | SEQ ID NO: 1814 |
| KHK_miR_1400 | SEQ ID NO: 400 | SEQ ID NO: 1815 |
| KHK_miR_1401 | SEQ ID NO: 401 | SEQ ID NO: 1816 |
| KHK_miR_1402 | SEQ ID NO: 402 | SEQ ID NO: 1817 |
| KHK_miR_1403 | SEQ ID NO: 403 | SEQ ID NO: 1818 |
| KHK_miR_1404 | SEQ ID NO: 404 | SEQ ID NO: 1819 |
| KHK_miR_1405 | SEQ ID NO: 405 | SEQ ID NO: 1820 |
| KHK_miR_1406 | SEQ ID NO: 406 | SEQ ID NO: 1821 |
| KHK_miR_1407 | SEQ ID NO: 407 | SEQ ID NO: 1822 |
| KHK_miR_1408 | SEQ ID NO: 408 | SEQ ID NO: 1823 |
| KHK_miR_1409 | SEQ ID NO: 409 | SEQ ID NO: 1824 |
| KHK_miR_1410 | SEQ ID NO: 410 | SEQ ID NO: 1825 |
| KHK_miR_1411 | SEQ ID NO: 411 | SEQ ID NO: 1826 |
| KHK_miR_1412 | SEQ ID NO: 412 | SEQ ID NO: 1827 |
| | | SEQ ID NO: 1828 |
| KHK_miR_1413 | SEQ ID NO: 413 | SEQ ID NO: 1829 |
| KHK_miR_1414 | SEQ ID NO: 414 | SEQ ID NO: 1830 |
| KHK_miR_1415 | SEQ ID NO: 415 | SEQ ID NO: 1831 |
| KHK_miR_1416 | SEQ ID NO: 416 | SEQ ID NO: 1832 |
| KHK_miR_1417 | SEQ ID NO: 417 | SEQ ID NO: 1833 |
| KHK_miR_1418 | SEQ ID NO: 418 | SEQ ID NO: 1834 |
| KHK_miR_1419 | SEQ ID NO: 419 | SEQ ID NO: 1835 |
| KHK_miR_1420 | SEQ ID NO: 420 | SEQ ID NO: 1836 |
| KHK_miR_1421 | SEQ ID NO: 421 | SEQ ID NO: 1837 |
| | | SEQ ID NO: 1838 |
| KHK_miR_1422 | SEQ ID NO: 422 | SEQ ID NO: 1839 |
| KHK_miR_1423 | SEQ ID NO: 423 | SEQ ID NO: 1840 |
| KHK_miR_1424 | SEQ ID NO: 424 | SEQ ID NO: 1841 |
| KHK_miR_1425 | SEQ ID NO: 425 | SEQ ID NO: 1842 |
| | | SEQ ID NO: 1843 |

TABLE 1-14

| nucleic acid | | micro-RNA |
|---|---|---|
| name | micro-RNA | precursor |
| KHK_miR_1426 | SEQ ID NO: 426 | SEQ ID NO: 1844 |
| KHK_miR_1427 | SEQ ID NO: 427 | SEQ ID NO: 1845 |
| KHK_miR_1428 | SEQ ID NO: 428 | SEQ ID NO: 1846 |
| KHK_miR_1429 | SEQ ID NO: 429 | SEQ ID NO: 1847 |
| KHK_miR_1430 | SEQ ID NO: 430 | SEQ ID NO: 1848 |
| KHK_miR_1431 | SEQ ID NO: 431 | SEQ ID NO: 1849 |
| | | SEQ ID NO: 1850 |
| KHK_miR_1432 | SEQ ID NO: 432 | SEQ ID NO: 1851 |
| KHK_miR_1433 | SEQ ID NO: 433 | SEQ ID NO: 1852 |
| KHK_miR_1434 | SEQ ID NO: 434 | SEQ ID NO: 1853 |
| KHK_miR_1435 | SEQ ID NO: 435 | SEQ ID NO: 1854 |
| KHK_miR_1436 | SEQ ID NO: 436 | SEQ ID NO: 1855 |
| KHK_miR_1437 | SEQ ID NO: 437 | SEQ ID NO: 1856 |
| KHK_miR_1438 | SEQ ID NO: 438 | SEQ ID NO: 1857 |
| KHK_miR_1439 | SEQ ID NO: 439 | SEQ ID NO: 1858 |
| KHK_miR_1440 | SEQ ID NO: 440 | SEQ ID NO: 1859 |
| KHK_miR_1441 | SEQ ID NO: 441 | SEQ ID NO: 1860 |
| KHK_miR_1442 | SEQ ID NO: 442 | SEQ ID NO: 1861 |
| KHK_miR_1443 | SEQ ID NO: 443 | SEQ ID NO: 1862 |
| KHK_miR_1444 | SEQ ID NO: 444 | SEQ ID NO: 1863 |
| KHK_miR_1445 | SEQ ID NO: 445 | SEQ ID NO: 1864 |
| KHK_miR_1446 | SEQ ID NO: 446 | SEQ ID NO: 1865 |
| | | SEQ ID NO: 1866 |
| KHK_miR_1447 | SEQ ID NO: 447 | SEQ ID NO: 1867 |
| KHK_miR_1448 | SEQ ID NO: 448 | SEQ ID NO: 1868 |
| KHK_miR_1449 | SEQ ID NO: 449 | SEQ ID NO: 1869 |
| KHK_miR_1450 | SEQ ID NO: 450 | SEQ ID NO: 1870 |
| KHK_miR_1451 | SEQ ID NO: 451 | SEQ ID NO: 1871 |
| KHK_miR_1452 | SEQ ID NO: 452 | SEQ ID NO: 1872 |
| KHK_miR_1453 | SEQ ID NO: 453 | SEQ ID NO: 1873 |
| KHK_miR_1454 | SEQ ID NO: 454 | SEQ ID NO: 1874 |
| KHK_miR_1455 | SEQ ID NO: 455 | SEQ ID NO: 1875 |
| KHK_miR_1456 | SEQ ID NO: 456 | SEQ ID NO: 1876 |
| KHK_miR_1457 | SEQ ID NO: 457 | SEQ ID NO: 1877 |
| KHK_miR_1458 | SEQ ID NO: 458 | SEQ ID NO: 1878 |
| KHK_miR_1459 | SEQ ID NO: 459 | SEQ ID NO: 1879 |
| KHK_miR_1460 | SEQ ID NO: 460 | SEQ ID NO: 1880 |
| KHK_miR_1461 | SEQ ID NO: 461 | SEQ ID NO: 1881 |
| KHK_miR_1462 | SEQ ID NO: 462 | SEQ ID NO: 1882 |

TABLE 1-15

| nucleic acid | | micro-RNA |
|---|---|---|
| name | micro-RNA | precursor |
| KHK_miR_1463 | SEQ ID NO: 463 | SEQ ID NO: 1883 |
| KHK_miR_1464 | SEQ ID NO: 464 | SEQ ID NO: 1884 |
| KHK_miR_1465 | SEQ ID NO: 465 | SEQ ID NO: 1885 |
| KHK_miR_1466 | SEQ ID NO: 466 | SEQ ID NO: 1886 |
| KHK_miR_1467 | SEQ ID NO: 467 | SEQ ID NO: 1887 |
| KHK_miR_1468 | SEQ ID NO: 468 | SEQ ID NO: 1888 |
| KHK_miR_1469 | SEQ ID NO: 469 | SEQ ID NO: 1889 |
| KHK_miR_1470 | SEQ ID NO: 470 | SEQ ID NO: 1890 |
| KHK_miR_1471 | SEQ ID NO: 471 | SEQ ID NO: 1891 |
| KHK_miR_1472 | SEQ ID NO: 472 | SEQ ID NO: 1892 |
| KHK_miR_1473 | SEQ ID NO: 473 | SEQ ID NO: 1893 |
| KHK_miR_1474 | SEQ ID NO: 474 | SEQ ID NO: 1894 |
| KHK_miR_1475 | SEQ ID NO: 475 | SEQ ID NO: 1895 |
| KHK_miR_1476 | SEQ ID NO: 476 | SEQ ID NO: 1896 |
| KHK_miR_1477 | SEQ ID NO: 477 | SEQ ID NO: 1897 |
| KHK_miR_1478 | SEQ ID NO: 478 | SEQ ID NO: 1898 |
| KHK_miR_1479 | SEQ ID NO: 479 | SEQ ID NO: 1899 |
| KHK_miR_1480 | SEQ ID NO: 480 | SEQ ID NO: 1900 |
| KHK_miR_1481 | SEQ ID NO: 481 | SEQ ID NO: 1901 |
| KHK_miR_1482 | SEQ ID NO: 482 | SEQ ID NO: 1902 |
| KHK_miR_1483 | SEQ ID NO: 483 | SEQ ID NO: 1903 |
| KHK_miR_1484 | SEQ ID NO: 484 | SEQ ID NO: 1904 |
| KHK_miR_1485 | SEQ ID NO: 485 | SEQ ID NO: 1905 |
| KHK_miR_1486 | SEQ ID NO: 486 | SEQ ID NO: 1906 |
| KHK_miR_1487 | SEQ ID NO: 487 | SEQ ID NO: 1907 |
| KHK_miR_1488 | SEQ ID NO: 488 | SEQ ID NO: 1908 |
| KHK_miR_1489 | SEQ ID NO: 489 | SEQ ID NO: 1909 |
| KHK_miR_1490 | SEQ ID NO: 490 | SEQ ID NO: 1910 |
| KHK_miR_1491 | SEQ ID NO: 491 | SEQ ID NO: 1911 |
| KHK_miR_1492 | SEQ ID NO: 492 | SEQ ID NO: 1912 |
| KHK_miR_1493 | SEQ ID NO: 493 | SEQ ID NO: 1913 |
| KHK_miR_1494 | SEQ ID NO: 494 | SEQ ID NO: 1914 |
| KHK_miR_1495 | SEQ ID NO: 495 | SEQ ID NO: 1915 |
| KHK_miR_1496 | SEQ ID NO: 496 | SEQ ID NO: 1916 |
| KHK_miR_1497 | SEQ ID NO: 497 | SEQ ID NO: 1917 |
| KHK_miR_1498 | SEQ ID NO: 498 | SEQ ID NO: 1918 |
| KHK_miR_1499 | SEQ ID NO: 499 | SEQ ID NO: 1919 |
| | | SEQ ID NO: 1920 |
| | | SEQ ID NO: 1921 |

TABLE 1-16

| nucleic acid | | micro-RNA |
|---|---|---|
| name | micro-RNA | precursor |
| KHK_miR_1500 | SEQ ID NO: 500 | SEQ ID NO: 1922 |
| KHK_miR_1501 | SEQ ID NO: 501 | SEQ ID NO: 1923 |
| KHK_miR_1502 | SEQ ID NO: 502 | SEQ ID NO: 1924 |
| KHK_miR_1503 | SEQ ID NO: 503 | SEQ ID NO: 1925 |
| KHK_miR_1504 | SEQ ID NO: 504 | SEQ ID NO: 1926 |
| KHK_miR_1505 | SEQ ID NO: 505 | SEQ ID NO: 1927 |
| KHK_miR_1506 | SEQ ID NO: 506 | SEQ ID NO: 1928 |
| KHK_miR_1507 | SEQ ID NO: 507 | SEQ ID NO: 1929 |
| | | SEQ ID NO: 1930 |
| KHK_miR_1508 | SEQ ID NO: 508 | SEQ ID NO: 1931 |
| | | SEQ ID NO: 1932 |
| KHK_miR_1509 | SEQ ID NO: 509 | SEQ ID NO: 1933 |
| KHK_miR_1510 | SEQ ID NO: 510 | SEQ ID NO: 1934 |
| KHK_miR_1511 | SEQ ID NO: 511 | SEQ ID NO: 1935 |
| KHK_miR_1512 | SEQ ID NO: 512 | SEQ ID NO: 1936 |
| KHK_miR_1513 | SEQ ID NO: 513 | SEQ ID NO: 1937 |
| KHK_miR_1514 | SEQ ID NO: 514 | SEQ ID NO: 1938 |
| KHK_miR_1515 | SEQ ID NO: 515 | SEQ ID NO: 1939 |
| KHK_miR_1516 | SEQ ID NO: 516 | SEQ ID NO: 1940 |
| KHK_miR_1517 | SEQ ID NO: 517 | SEQ ID NO: 1941 |
| KHK_miR_1518 | SEQ ID NO: 518 | SEQ ID NO: 1942 |
| KHK_miR_1519 | SEQ ID NO: 519 | SEQ ID NO: 1943 |
| | | SEQ ID NO: 1944 |
| KHK_miR_1520 | SEQ ID NO: 520 | SEQ ID NO: 1945 |
| KHK_miR_1521 | SEQ ID NO: 521 | SEQ ID NO: 1946 |
| KHK_miR_1522 | SEQ ID NO: 522 | SEQ ID NO: 1947 |
| KHK_miR_1523 | SEQ ID NO: 523 | SEQ ID NO: 1948 |
| KHK_miR_1524 | SEQ ID NO: 524 | SEQ ID NO: 1949 |
| KHK_miR_1525 | SEQ ID NO: 525 | SEQ ID NO: 1950 |
| KHK_miR_1526 | SEQ ID NO: 526 | SEQ ID NO: 1951 |
| KHK_miR_1527 | SEQ ID NO: 527 | SEQ ID NO: 1952 |
| KHK_miR_1528 | SEQ ID NO: 528 | SEQ ID NO: 1953 |
| KHK_miR_1529 | SEQ ID NO: 529 | SEQ ID NO: 1954 |
| KHK_miR_1530 | SEQ ID NO: 530 | SEQ ID NO: 1955 |
| KHK_miR_1531 | SEQ ID NO: 531 | SEQ ID NO: 1956 |
| KHK_miR_1532 | SEQ ID NO: 532 | SEQ ID NO: 1957 |
| | | SEQ ID NO: 1958 |
| KHK_miR_1533 | SEQ ID NO: 533 | SEQ ID NO: 1959 |
| KHK_miR_1534 | SEQ ID NO: 534 | SEQ ID NO: 1960 |

TABLE 1-17

| nucleic acid | | micro-RNA |
|---|---|---|
| name | micro-RNA | precursor |
| KHK_miR_1535 | SEQ ID NO: 535 | SEQ ID NO: 1961 |
| KHK_miR_1536 | SEQ ID NO: 536 | SEQ ID NO: 1962 |
| KHK_miR_1537 | SEQ ID NO: 537 | SEQ ID NO: 1963 |
| KHK_miR_1538 | SEQ ID NO: 538 | SEQ ID NO: 1964 |
| KHK_miR_1539 | SEQ ID NO: 539 | SEQ ID NO: 1965 |
| KHK_miR_1540 | SEQ ID NO: 540 | SEQ ID NO: 1966 |
| KHK_miR_1541 | SEQ ID NO: 541 | SEQ ID NO: 1967 |
| KHK_miR_1542 | SEQ ID NO: 542 | SEQ ID NO: 1968 |
| KHK_miR_1543 | SEQ ID NO: 543 | SEQ ID NO: 1969 |
| KHK_miR_1544 | SEQ ID NO: 544 | SEQ ID NO: 1970 |
| KHK_miR_1545 | SEQ ID NO: 545 | SEQ ID NO: 1971 |
| KHK_miR_1546 | SEQ ID NO: 546 | SEQ ID NO: 1972 |
| KHK_miR_1547 | SEQ ID NO: 547 | SEQ ID NO: 1973 |
| KHK_miR_1548 | SEQ ID NO: 548 | SEQ ID NO: 1974 |
| KHK_miR_1549 | SEQ ID NO: 549 | SEQ ID NO: 1975 |
| KHK_miR_1550 | SEQ ID NO: 550 | SEQ ID NO: 1976 |
| KHK_miR_1551 | SEQ ID NO: 551 | SEQ ID NO: 1977 |
| KHK_miR_1552 | SEQ ID NO: 552 | SEQ ID NO: 1978 |
| KHK_miR_1553 | SEQ ID NO: 553 | SEQ ID NO: 1979 |
| KHK_miR_1554 | SEQ ID NO: 554 | SEQ ID NO: 1980 |
| KHK_miR_1555 | SEQ ID NO: 555 | SEQ ID NO: 1981 |
| KHK_miR_1556 | SEQ ID NO: 556 | SEQ ID NO: 1982 |
| KHK_miR_1557 | SEQ ID NO: 557 | SEQ ID NO: 1983 |
| | | SEQ ID NO: 1984 |
| KHK_miR_1558 | SEQ ID NO: 558 | SEQ ID NO: 1985 |
| KHK_miR_1559 | SEQ ID NO: 559 | SEQ ID NO: 1986 |
| KHK_miR_1560 | SEQ ID NO: 560 | SEQ ID NO: 1987 |
| KHK_miR_1561 | SEQ ID NO: 561 | SEQ ID NO: 1988 |
| KHK_miR_1562 | SEQ ID NO: 562 | SEQ ID NO: 1989 |
| KHK_miR_1563 | SEQ ID NO: 563 | SEQ ID NO: 1990 |
| KHK_miR_1564 | SEQ ID NO: 564 | SEQ ID NO: 1991 |
| KHK_miR_1565 | SEQ ID NO: 565 | SEQ ID NO: 1992 |
| KHK_miR_1566 | SEQ ID NO: 566 | SEQ ID NO: 1993 |
| KHK_miR_1567 | SEQ ID NO: 567 | SEQ ID NO: 1994 |
| KHK_miR_1568 | SEQ ID NO: 568 | SEQ ID NO: 1995 |
| | | SEQ ID NO: 1996 |
| | | SEQ ID NO: 1997 |
| KHK_miR_1569 | SEQ ID NO: 569 | SEQ ID NO: 1998 |
| KHK_miR_1570 | SEQ ID NO: 570 | SEQ ID NO: 1999 |

TABLE 1-18

| nucleic acid | | micro-RNA |
|---|---|---|
| name | micro-RNA | precursor |
| KHK_miR_1571 | SEQ ID NO: 571 | SEQ ID NO: 2000 |
| KHK_miR_1572 | SEQ ID NO: 572 | SEQ ID NO: 2001 |
| KHK_miR_1573 | SEQ ID NO: 573 | SEQ ID NO: 2002 |
| KHK_miR_1574 | SEQ ID NO: 574 | SEQ ID NO: 2003 |
| KHK_miR_1575 | SEQ ID NO: 575 | SEQ ID NO: 2004 |
| KHK_miR_1576 | SEQ ID NO: 576 | SEQ ID NO: 2005 |
| | | SEQ ID NO: 2006 |
| | | SEQ ID NO: 2007 |
| | | SEQ ID NO: 2008 |
| | | SEQ ID NO: 2009 |
| KHK_miR_1577 | SEQ ID NO: 577 | SEQ ID NO: 2010 |
| KHK_miR_1578 | SEQ ID NO: 578 | SEQ ID NO: 2011 |
| KHK_miR_1579 | SEQ ID NO: 579 | SEQ ID NO: 2012 |
| KHK_miR_1580 | SEQ ID NO: 580 | SEQ ID NO: 2013 |
| KHK_miR_1581 | SEQ ID NO: 581 | SEQ ID NO: 2014 |
| KHK_miR_1582 | SEQ ID NO: 582 | SEQ ID NO: 2015 |
| KHK_miR_1583 | SEQ ID NO: 583 | SEQ ID NO: 2016 |
| | | SEQ ID NO: 2017 |
| | | SEQ ID NO: 2018 |
| KHK_miR_1584 | SEQ ID NO: 584 | SEQ ID NO: 2019 |
| KHK_miR_1585 | SEQ ID NO: 585 | SEQ ID NO: 2020 |
| KHK_miR_1586 | SEQ ID NO: 586 | SEQ ID NO: 2021 |
| KHK_miR_1587 | SEQ ID NO: 587 | SEQ ID NO: 2022 |
| KHK_miR_1588 | SEQ ID NO: 588 | SEQ ID NO: 2023 |
| KHK_miR_1589 | SEQ ID NO: 589 | SEQ ID NO: 2024 |
| KHK_miR_1590 | SEQ ID NO: 590 | SEQ ID NO: 2025 |
| KHK_miR_1591 | SEQ ID NO: 591 | SEQ ID NO: 2026 |
| KHK_miR_1592 | SEQ ID NO: 592 | SEQ ID NO: 2027 |
| KHK_miR_1593 | SEQ ID NO: 593 | SEQ ID NO: 2028 |
| KHK_miR_1594 | SEQ ID NO: 594 | SEQ ID NO: 2029 |
| KHK_miR_1595 | SEQ ID NO: 595 | SEQ ID NO: 2030 |
| KHK_miR_1596 | SEQ ID NO: 596 | SEQ ID NO: 2031 |
| KHK_miR_1597 | SEQ ID NO: 597 | SEQ ID NO: 2032 |
| KHK_miR_1598 | SEQ ID NO: 598 | SEQ ID NO: 2033 |
| KHK_miR_1599 | SEQ ID NO: 599 | SEQ ID NO: 2034 |
| KHK_miR_1600 | SEQ ID NO: 600 | SEQ ID NO: 2035 |
| | | SEQ ID NO: 2036 |
| | | SEQ ID NO: 2037 |
| KHK_miR_1601 | SEQ ID NO: 601 | SEQ ID NO: 2038 |

TABLE 1-19

| nucleic acid | | micro-RNA |
|---|---|---|
| name | micro-RNA | precursor |
| KHK_miR_1602 | SEQ ID NO: 602 | SEQ ID NO: 2039 |
| KHK_miR_1603 | SEQ ID NO: 603 | SEQ ID NO: 2040 |
| KHK_miR_1604 | SEQ ID NO: 604 | SEQ ID NO: 2041 |
| KHK_miR_1605 | SEQ ID NO: 605 | SEQ ID NO: 2042 |
| KHK_miR_1606 | SEQ ID NO: 606 | SEQ ID NO: 2043 |
| KHK_miR_1607 | SEQ ID NO: 607 | SEQ ID NO: 2044 |
| KHK_miR_1608 | SEQ ID NO: 608 | SEQ ID NO: 2045 |
| KHK_miR_1609 | SEQ ID NO: 609 | SEQ ID NO: 2046 |
| KHK_miR_1610 | SEQ ID NO: 610 | SEQ ID NO: 2047 |
| KHK_miR_1611 | SEQ ID NO: 611 | SEQ ID NO: 2048 |
| KHK_miR_1612 | SEQ ID NO: 612 | SEQ ID NO: 2049 |
| KHK_miR_1613 | SEQ ID NO: 613 | SEQ ID NO: 2050 |
| KHK_miR_1614 | SEQ ID NO: 614 | SEQ ID NO: 2051 |
| KHK_miR_1615 | SEQ ID NO: 615 | SEQ ID NO: 2052 |
| KHK_miR_1616 | SEQ ID NO: 616 | SEQ ID NO: 2053 |
| KHK_miR_1617 | SEQ ID NO: 617 | SEQ ID NO: 2054 |
| KHK_miR_1618 | SEQ ID NO: 618 | SEQ ID NO: 2055 |
| KHK_miR_1619 | SEQ ID NO: 619 | SEQ ID NO: 2056 |
| KHK_miR_1620 | SEQ ID NO: 620 | SEQ ID NO: 2057 |
| KHK_miR_1621 | SEQ ID NO: 621 | SEQ ID NO: 2058 |
| KHK_miR_1622 | SEQ ID NO: 622 | SEQ ID NO: 2059 |
| KHK_miR_1623 | SEQ ID NO: 623 | SEQ ID NO: 2060 |
| KHK_miR_1624 | SEQ ID NO: 624 | SEQ ID NO: 2061 |
| KHK_miR_1625 | SEQ ID NO: 625 | SEQ ID NO: 2062 |
| KHK_miR_1626 | SEQ ID NO: 626 | SEQ ID NO: 2063 |
| KHK_miR_1627 | SEQ ID NO: 627 | SEQ ID NO: 2064 |
| KHK_miR_1628 | SEQ ID NO: 628 | SEQ ID NO: 2065 |
| KHK_miR_1629 | SEQ ID NO: 629 | SEQ ID NO: 2066 |
| KHK_miR_1630 | SEQ ID NO: 630 | SEQ ID NO: 2067 |
| KHK_miR_1631 | SEQ ID NO: 631 | SEQ ID NO: 2068 |
| KHK_miR_1632 | SEQ ID NO: 632 | SEQ ID NO: 2069 |
| KHK_miR_1633 | SEQ ID NO: 633 | SEQ ID NO: 2070 |
| KHK_miR_1634 | SEQ ID NO: 634 | SEQ ID NO: 2071 |
| KHK_miR_1635 | SEQ ID NO: 635 | SEQ ID NO: 2072 |
| KHK_miR_1636 | SEQ ID NO: 636 | SEQ ID NO: 2073 |
| KHK_miR_1637 | SEQ ID NO: 637 | SEQ ID NO: 2074 |
| KHK_miR_1638 | SEQ ID NO: 638 | SEQ ID NO: 2075 |
| | | SEQ ID NO: 2076 |
| | | SEQ ID NO: 2077 |

TABLE 1-20

| nucleic acid | | micro-RNA |
|---|---|---|
| name | micro-RNA | precursor |
| | | SEQ ID NO: 2078 |
| KHK_miR_1639 | SEQ ID NO: 639 | SEQ ID NO: 2079 |
| KHK_miR_1640 | SEQ ID NO: 640 | SEQ ID NO: 2080 |
| KHK_miR_1641 | SEQ ID NO: 641 | SEQ ID NO: 2081 |
| KHK_miR_1642 | SEQ ID NO: 642 | SEQ ID NO: 2082 |
| | | SEQ ID NO: 2083 |
| KHK_miR_1643 | SEQ ID NO: 643 | SEQ ID NO: 2084 |
| KHK_miR_1644 | SEQ ID NO: 644 | SEQ ID NO: 2085 |
| KHK_miR_1645 | SEQ ID NO: 645 | SEQ ID NO: 2086 |
| KHK_miR_1646 | SEQ ID NO: 646 | SEQ ID NO: 2087 |
| KHK_miR_1647 | SEQ ID NO: 647 | SEQ ID NO: 2088 |
| KHK_miR_1648 | SEQ ID NO: 648 | SEQ ID NO: 2089 |
| KHK_miR_1649 | SEQ ID NO: 649 | SEQ ID NO: 2090 |
| KHK_miR_1650 | SEQ ID NO: 650 | SEQ ID NO: 2091 |
| KHK_miR_1651 | SEQ ID NO: 651 | SEQ ID NO: 2092 |
| KHK_miR_1652 | SEQ ID NO: 652 | SEQ ID NO: 2093 |
| KHK_miR_1653 | SEQ ID NO: 653 | SEQ ID NO: 2094 |
| | | SEQ ID NO: 2095 |
| KHK_miR_1654 | SEQ ID NO: 654 | SEQ ID NO: 2096 |
| KHK_miR_1655 | SEQ ID NO: 655 | SEQ ID NO: 2097 |
| KHK_miR_1656 | SEQ ID NO: 656 | SEQ ID NO: 2098 |
| KHK_miR_1657 | SEQ ID NO: 657 | SEQ ID NO: 2099 |
| KHK_miR_1658 | SEQ ID NO: 658 | SEQ ID NO: 2100 |
| | | SEQ ID NO: 2101 |
| KHK_miR_1659 | SEQ ID NO: 659 | SEQ ID NO: 2102 |
| KHK_miR_1660 | SEQ ID NO: 660 | SEQ ID NO: 2103 |
| KHK_miR_1661 | SEQ ID NO: 661 | SEQ ID NO: 2104 |
| KHK_miR_1662 | SEQ ID NO: 662 | SEQ ID NO: 2105 |
| KHK_miR_1663 | SEQ ID NO: 663 | SEQ ID NO: 2106 |
| KHK_miR_1664 | SEQ ID NO: 664 | SEQ ID NO: 2107 |
| KHK_miR_1665 | SEQ ID NO: 665 | SEQ ID NO: 2108 |
| KHK_miR_1666 | SEQ ID NO: 666 | SEQ ID NO: 2109 |
| KHK_miR_1667 | SEQ ID NO: 667 | SEQ ID NO: 2110 |
| KHK_miR_1668 | SEQ ID NO: 668 | SEQ ID NO: 2111 |
| KHK_miR_1669 | SEQ ID NO: 669 | SEQ ID NO: 2112 |
| KHK_miR_1670 | SEQ ID NO: 670 | SEQ ID NO: 2113 |
| KHK_miR_1671 | SEQ ID NO: 671 | SEQ ID NO: 2114 |
| KHK_miR_1672 | SEQ ID NO: 672 | SEQ ID NO: 2115 |
| KHK_miR_1673 | SEQ ID NO: 673 | SEQ ID NO: 2116 |

TABLE 1-21

| nucleic acid | | micro-RNA |
|---|---|---|
| name | micro-RNA | precursor |
| KHK_miR_1674 | SEQ ID NO: 674 | SEQ ID NO: 2117 |
| KHK_miR_1675 | SEQ ID NO: 675 | SEQ ID NO: 2118 |
| KHK_miR_1676 | SEQ ID NO: 676 | SEQ ID NO: 2119 |
| KHK_miR_1677 | SEQ ID NO: 677 | SEQ ID NO: 2120 |
| KHK_miR_1678 | SEQ ID NO: 678 | SEQ ID NO: 2121 |
| | | SEQ ID NO: 2122 |
| KHK_miR_1679 | SEQ ID NO: 679 | SEQ ID NO: 2123 |
| KHK_miR_1680 | SEQ ID NO: 680 | SEQ ID NO: 2124 |
| | | SEQ ID NO: 2125 |
| | | SEQ ID NO: 2126 |
| | | SEQ ID NO: 2127 |
| KHK_miR_1681 | SEQ ID NO: 681 | SEQ ID NO: 2128 |
| KHK_miR_1682 | SEQ ID NO: 682 | SEQ ID NO: 2129 |
| KHK_miR_1683 | SEQ ID NO: 683 | SEQ ID NO: 2130 |
| KHK_miR_1684 | SEQ ID NO: 684 | SEQ ID NO: 2131 |
| KHK_miR_1685 | SEQ ID NO: 685 | SEQ ID NO: 2132 |
| KHK_miR_1686 | SEQ ID NO: 686 | SEQ ID NO: 2133 |
| KHK_miR_1687 | SEQ ID NO: 687 | SEQ ID NO: 2134 |
| KHK_miR_1688 | SEQ ID NO: 688 | SEQ ID NO: 2135 |
| KHK_miR_1689 | SEQ ID NO: 689 | SEQ ID NO: 2136 |
| KHK_miR_1690 | SEQ ID NO: 690 | SEQ ID NO: 2137 |
| KHK_miR_1691 | SEQ ID NO: 691 | SEQ ID NO: 2138 |
| | | SEQ ID NO: 2139 |
| KHK_miR_1692 | SEQ ID NO: 692 | SEQ ID NO: 2140 |
| KHK_miR_1693 | SEQ ID NO: 693 | SEQ ID NO: 2141 |
| KHK_miR_1694 | SEQ ID NO: 694 | SEQ ID NO: 2142 |
| KHK_miR_1695 | SEQ ID NO: 695 | SEQ ID NO: 2143 |
| KHK_miR_1696 | SEQ ID NO: 696 | SEQ ID NO: 2144 |
| KHK_miR_1697 | SEQ ID NO: 697 | SEQ ID NO: 2145 |
| KHK_miR_1698 | SEQ ID NO: 698 | SEQ ID NO: 2146 |
| KHK_miR_1699 | SEQ ID NO: 699 | SEQ ID NO: 2147 |
| KHK_miR_1700 | SEQ ID NO: 700 | SEQ ID NO: 2148 |
| KHK_miR_1701 | SEQ ID NO: 701 | SEQ ID NO: 2149 |
| KHK_miR_1702 | SEQ ID NO: 702 | SEQ ID NO: 2150 |
| KHK_miR_1703 | SEQ ID NO: 703 | SEQ ID NO: 2151 |
| KHK_miR_1704 | SEQ ID NO: 704 | SEQ ID NO: 2152 |
| KHK_miR_1705 | SEQ ID NO: 705 | SEQ ID NO: 2153 |
| KHK_miR_1706 | SEQ ID NO: 706 | SEQ ID NO: 2154 |
| KHK_miR_1707 | SEQ ID NO: 707 | SEQ ID NO: 2155 |

TABLE 1-22

| nucleic acid | | micro-RNA |
|---|---|---|
| name | micro-RNA | precursor |
| KHK_miR_1708 | SEQ ID NO: 708 | SEQ ID NO: 2156 |
| KHK_miR_1709 | SEQ ID NO: 709 | SEQ ID NO: 2157 |
| KHK_miR_1710 | SEQ ID NO: 710 | SEQ ID NO: 2158 |
| KHK_miR_1711 | SEQ ID NO: 711 | SEQ ID NO: 2159 |
| KHK_miR_1712 | SEQ ID NO: 712 | SEQ ID NO: 2160 |
| KHK_miR_1713 | SEQ ID NO: 713 | SEQ ID NO: 2161 |
| KHK_miR_1714 | SEQ ID NO: 714 | SEQ ID NO: 2162 |
| KHK_miR_1715 | SEQ ID NO: 715 | SEQ ID NO: 2163 |
| KHK_miR_1716 | SEQ ID NO: 716 | SEQ ID NO: 2164 |
| KHK_miR_1717 | SEQ ID NO: 717 | SEQ ID NO: 2165 |
| KHK_miR_1718 | SEQ ID NO: 718 | SEQ ID NO: 2166 |
| KHK_miR_1719 | SEQ ID NO: 719 | SEQ ID NO: 2167 |
| KHK_miR_1720 | SEQ ID NO: 720 | SEQ ID NO: 2168 |
| KHK_miR_1721 | SEQ ID NO: 721 | SEQ ID NO: 2169 |
| KHK_miR_1722 | SEQ ID NO: 722 | SEQ ID NO: 2170 |
| KHK_miR_1723 | SEQ ID NO: 723 | SEQ ID NO: 2171 |
| KHK_miR_1724 | SEQ ID NO: 724 | SEQ ID NO: 2172 |
| KHK_miR_1725 | SEQ ID NO: 725 | SEQ ID NO: 2173 |
| KHK_miR_1726 | SEQ ID NO: 726 | SEQ ID NO: 2174 |
| KHK_miR_1727 | SEQ ID NO: 727 | SEQ ID NO: 2175 |
| KHK_miR_1728 | SEQ ID NO: 728 | SEQ ID NO: 2176 |
| KHK_miR_1729 | SEQ ID NO: 729 | SEQ ID NO: 2177 |
| KHK_miR_1730 | SEQ ID NO: 730 | SEQ ID NO: 2178 |
| KHK_miR_1731 | SEQ ID NO: 731 | SEQ ID NO: 2179 |
| KHK_miR_1732 | SEQ ID NO: 732 | SEQ ID NO: 2180 |
| KHK_miR_1733 | SEQ ID NO: 733 | SEQ ID NO: 2181 |
| KHK_miR_1734 | SEQ ID NO: 734 | SEQ ID NO: 2182 |
| KHK_miR_1735 | SEQ ID NO: 735 | SEQ ID NO: 2183 |
| KHK_miR_1736 | SEQ ID NO: 736 | SEQ ID NO: 2184 |
| KHK_miR_1737 | SEQ ID NO: 737 | SEQ ID NO: 2185 |
| KHK_miR_1738 | SEQ ID NO: 738 | SEQ ID NO: 2186 |
| KHK_miR_1739 | SEQ ID NO: 739 | SEQ ID NO: 2187 |
| KHK_miR_1740 | SEQ ID NO: 740 | SEQ ID NO: 2188 |
| KHK_miR_1741 | SEQ ID NO: 741 | SEQ ID NO: 2189 |
| KHK_miR_1742 | SEQ ID NO: 742 | SEQ ID NO: 2190 |
| | | SEQ ID NO: 2191 |
| | | SEQ ID NO: 2192 |
| KHK_miR_1743 | SEQ ID NO: 743 | SEQ ID NO: 2193 |
| KHK_miR_1744 | SEQ ID NO: 744 | SEQ ID NO: 2194 |

TABLE 1-23

| nucleic acid | | micro-RNA |
|---|---|---|
| name | micro-RNA | precursor |
| KHK_miR_1745 | SEQ ID NO: 745 | SEQ ID NO: 2195 |
| KHK_miR_1746 | SEQ ID NO: 746 | SEQ ID NO: 2196 |
| | | SEQ ID NO: 2197 |
| KHK_miR_1747 | SEQ ID NO: 747 | SEQ ID NO: 2198 |
| KHK_miR_1748 | SEQ ID NO: 748 | SEQ ID NO: 2199 |
| KHK_miR_1749 | SEQ ID NO: 749 | SEQ ID NO: 2200 |
| KHK_miR_1750 | SEQ ID NO: 750 | SEQ ID NO: 2201 |
| KHK_miR_1751 | SEQ ID NO: 751 | SEQ ID NO: 2202 |
| KHK_miR_1752 | SEQ ID NO: 752 | SEQ ID NO: 2203 |
| KHK_miR_1753 | SEQ ID NO: 753 | SEQ ID NO: 2204 |
| KHK_miR_1754 | SEQ ID NO: 754 | SEQ ID NO: 2205 |
| KHK_miR_1755 | SEQ ID NO: 755 | SEQ ID NO: 2206 |
| KHK_miR_1756 | SEQ ID NO: 756 | SEQ ID NO: 2207 |
| KHK_miR_1757 | SEQ ID NO: 757 | SEQ ID NO: 2208 |
| KHK_miR_1758 | SEQ ID NO: 758 | SEQ ID NO: 2209 |
| KHK_miR_1759 | SEQ ID NO: 759 | SEQ ID NO: 2210 |
| KHK_miR_1760 | SEQ ID NO: 760 | SEQ ID NO: 2211 |
| KHK_miR_1761 | SEQ ID NO: 761 | SEQ ID NO: 2212 |
| KHK_miR_1762 | SEQ ID NO: 762 | SEQ ID NO: 2213 |
| KHK_miR_1763 | SEQ ID NO: 763 | SEQ ID NO: 2214 |
| KHK_miR_1764 | SEQ ID NO: 764 | SEQ ID NO: 2215 |
| KHK_miR_1765 | SEQ ID NO: 765 | SEQ ID NO: 2216 |
| KHK_miR_1766 | SEQ ID NO: 766 | SEQ ID NO: 2217 |
| KHK_miR_1767 | SEQ ID NO: 767 | SEQ ID NO: 2218 |
| KHK_miR_1768 | SEQ ID NO: 768 | SEQ ID NO: 2219 |
| | | SEQ ID NO: 2220 |
| | | SEQ ID NO: 2221 |
| | | SEQ ID NO: 2222 |
| KHK_miR_1769 | SEQ ID NO: 769 | SEQ ID NO: 2223 |
| KHK_miR_1770 | SEQ ID NO: 770 | SEQ ID NO: 2224 |
| KHK_miR_1771 | SEQ ID NO: 771 | SEQ ID NO: 2225 |
| KHK_miR_1772 | SEQ ID NO: 772 | SEQ ID NO: 2226 |
| KHK_miR_1773 | SEQ ID NO: 773 | SEQ ID NO: 2227 |
| KHK_miR_1774 | SEQ ID NO: 774 | SEQ ID NO: 2228 |
| KHK_miR_1775 | SEQ ID NO: 775 | SEQ ID NO: 2229 |
| | | SEQ ID NO: 2230 |
| | | SEQ ID NO: 2231 |
| KHK_miR_1776 | SEQ ID NO: 776 | SEQ ID NO: 2232 |
| KHK_miR_1777 | SEQ ID NO: 777 | SEQ ID NO: 2233 |

TABLE 1-24

| nucleic acid | | micro-RNA |
|---|---|---|
| name | micro-RNA | precursor |
| KHK_miR_1778 | SEQ ID NO: 778 | SEQ ID NO: 2234 |
| KHK_miR_1779 | SEQ ID NO: 779 | SEQ ID NO: 2235 |
| KHK_miR_1780 | SEQ ID NO: 780 | SEQ ID NO: 2236 |
| KHK_miR_1781 | SEQ ID NO: 781 | SEQ ID NO: 2237 |
| KHK_miR_1782 | SEQ ID NO: 782 | SEQ ID NO: 2238 |
| KHK_miR_1783 | SEQ ID NO: 783 | SEQ ID NO: 2239 |
| KHK_miR_1784 | SEQ ID NO: 784 | SEQ ID NO: 2240 |
| | | SEQ ID NO: 2241 |
| | | SEQ ID NO: 2242 |
| | | SEQ ID NO: 2243 |
| | | SEQ ID NO: 2244 |
| | | SEQ ID NO: 2245 |
| | | SEQ ID NO: 2246 |
| | | SEQ ID NO: 2247 |
| KHK_miR_1785 | SEQ ID NO: 785 | SEQ ID NO: 2248 |
| KHK_miR_1786 | SEQ ID NO: 786 | SEQ ID NO: 2249 |
| KHK_miR_1787 | SEQ ID NO: 787 | SEQ ID NO: 2250 |
| KHK_miR_1788 | SEQ ID NO: 788 | SEQ ID NO: 2251 |
| KHK_miR_1789 | SEQ ID NO: 789 | SEQ ID NO: 2252 |
| KHK_miR_1790 | SEQ ID NO: 790 | SEQ ID NO: 2253 |
| KHK_miR_1791 | SEQ ID NO: 791 | SEQ ID NO: 2254 |
| KHK_miR_1792 | SEQ ID NO: 792 | SEQ ID NO: 2255 |
| KHK_miR_1793 | SEQ ID NO: 793 | SEQ ID NO: 2256 |
| KHK_miR_1794 | SEQ ID NO: 794 | SEQ ID NO: 2257 |
| KHK_miR_1795 | SEQ ID NO: 795 | SEQ ID NO: 2258 |
| KHK_miR_1796 | SEQ ID NO: 796 | SEQ ID NO: 2259 |
| KHK_miR_1797 | SEQ ID NO: 797 | SEQ ID NO: 2260 |
| KHK_miR_1798 | SEQ ID NO: 798 | SEQ ID NO: 2261 |
| KHK_miR_1799 | SEQ ID NO: 799 | SEQ ID NO: 2262 |
| KHK_miR_1800 | SEQ ID NO: 800 | SEQ ID NO: 2263 |
| KHK_miR_1801 | SEQ ID NO: 801 | SEQ ID NO: 2264 |
| KHK_miR_1802 | SEQ ID NO: 802 | SEQ ID NO: 2265 |
| | | SEQ ID NO: 2266 |
| KHK_miR_1803 | SEQ ID NO: 803 | SEQ ID NO: 2267 |
| KHK_miR_1804 | SEQ ID NO: 804 | SEQ ID NO: 2268 |
| KHK_miR_1805 | SEQ ID NO: 805 | SEQ ID NO: 2269 |
| KHK_miR_1806 | SEQ ID NO: 806 | SEQ ID NO: 2270 |
| KHK_miR_1807 | SEQ ID NO: 807 | SEQ ID NO: 2271 |
| KHK_miR_1808 | SEQ ID NO: 808 | SEQ ID NO: 2272 |

TABLE 1-25

| nucleic acid | | micro-RNA |
|---|---|---|
| name | micro-RNA | precursor |
| KHK_miR_1809 | SEQ ID NO: 809 | SEQ ID NO: 2273 |
| KHK_miR_1810 | SEQ ID NO: 810 | SEQ ID NO: 2274 |
| KHK_miR_1811 | SEQ ID NO: 811 | SEQ ID NO: 2275 |
| KHK_miR_1812 | SEQ ID NO: 812 | SEQ ID NO: 2276 |
| KHK_miR_1813 | SEQ ID NO: 813 | SEQ ID NO: 2277 |
| KHK_miR_1814 | SEQ ID NO: 814 | SEQ ID NO: 2278 |
| KHK_miR_1815 | SEQ ID NO: 815 | SEQ ID NO: 2279 |
| KHK_miR_1816 | SEQ ID NO: 816 | SEQ ID NO: 2280 |
| KHK_miR_1817 | SEQ ID NO: 817 | SEQ ID NO: 2281 |
| KHK_miR_1818 | SEQ ID NO: 818 | SEQ ID NO: 2282 |
| KHK_miR_1819 | SEQ ID NO: 819 | SEQ ID NO: 2283 |
| KHK_miR_1820 | SEQ ID NO: 820 | SEQ ID NO: 2284 |
| | | SEQ ID NO: 2285 |
| | | SEQ ID NO: 2286 |
| | | SEQ ID NO: 2287 |
| KHK_miR_1821 | SEQ ID NO: 821 | SEQ ID NO: 2288 |
| KHK_miR_1822 | SEQ ID NO: 822 | SEQ ID NO: 2289 |
| KHK_miR_1823 | SEQ ID NO: 823 | SEQ ID NO: 2290 |
| | | SEQ ID NO: 2291 |
| | | SEQ ID NO: 2292 |
| | | SEQ ID NO: 2293 |
| | | SEQ ID NO: 2294 |
| KHK_miR_1824 | SEQ ID NO: 824 | SEQ ID NO: 2295 |
| KHK_miR_1825 | SEQ ID NO: 825 | SEQ ID NO: 2296 |
| KHK_miR_1826 | SEQ ID NO: 826 | SEQ ID NO: 2297 |
| KHK_miR_1827 | SEQ ID NO: 827 | SEQ ID NO: 2298 |
| KHK_miR_1828 | SEQ ID NO: 828 | SEQ ID NO: 2299 |
| KHK_miR_1829 | SEQ ID NO: 829 | SEQ ID NO: 2300 |
| KHK_miR_1830 | SEQ ID NO: 830 | SEQ ID NO: 2301 |
| | | SEQ ID NO: 2302 |
| KHK_miR_1831 | SEQ ID NO: 831 | SEQ ID NO: 2303 |
| KHK_miR_1832 | SEQ ID NO: 832 | SEQ ID NO: 2304 |
| KHK_miR_1833 | SEQ ID NO: 833 | SEQ ID NO: 2305 |
| KHK_miR_1834 | SEQ ID NO: 834 | SEQ ID NO: 2306 |
| KHK_miR_1835 | SEQ ID NO: 835 | SEQ ID NO: 2307 |
| KHK_miR_1836 | SEQ ID NO: 836 | SEQ ID NO: 2308 |
| KHK_miR_1837 | SEQ ID NO: 837 | SEQ ID NO: 2309 |
| KHK_miR_1838 | SEQ ID NO: 838 | SEQ ID NO: 2310 |
| KHK_miR_1839 | SEQ ID NO: 839 | SEQ ID NO: 2311 |

TABLE 1-26

| nucleic acid | | micro-RNA |
|---|---|---|
| name | micro-RNA | precursor |
| KHK_miR_1840 | SEQ ID NO: 840 | SEQ ID NO: 2312 |
| KHK_miR_1841 | SEQ ID NO: 841 | SEQ ID NO: 2313 |
| KHK_miR_1842 | SEQ ID NO: 842 | SEQ ID NO: 2314 |
| KHK_miR_1843 | SEQ ID NO: 843 | SEQ ID NO: 2315 |
| KHK_miR_1844 | SEQ ID NO: 844 | SEQ ID NO: 2316 |
| KHK_miR_1845 | SEQ ID NO: 845 | SEQ ID NO: 2317 |
| KHK_miR_1846 | SEQ ID NO: 846 | SEQ ID NO: 2318 |
| KHK_miR_1847 | SEQ ID NO: 847 | SEQ ID NO: 2319 |
| KHK_miR_1848 | SEQ ID NO: 848 | SEQ ID NO: 2320 |
| KHK_miR_1849 | SEQ ID NO: 849 | SEQ ID NO: 2321 |
| KHK_miR_1850 | SEQ ID NO: 850 | SEQ ID NO: 2322 |
| KHK_miR_1851 | SEQ ID NO: 851 | SEQ ID NO: 2323 |
| KHK_miR_1852 | SEQ ID NO: 852 | SEQ ID NO: 2324 |
| | | SEQ ID NO: 2325 |
| KHK_miR_1853 | SEQ ID NO: 853 | SEQ ID NO: 2326 |
| KHK_miR_1854 | SEQ ID NO: 854 | SEQ ID NO: 2327 |
| KHK_miR_1855 | SEQ ID NO: 855 | SEQ ID NO: 2328 |
| KHK_miR_1856 | SEQ ID NO: 856 | SEQ ID NO: 2329 |
| KHK_miR_1857 | SEQ ID NO: 857 | SEQ ID NO: 2330 |
| KHK_miR_1858 | SEQ ID NO: 858 | SEQ ID NO: 2331 |
| KHK_miR_1859 | SEQ ID NO: 859 | SEQ ID NO: 2332 |
| KHK_miR_1860 | SEQ ID NO: 860 | SEQ ID NO: 2333 |
| KHK_miR_1861 | SEQ ID NO: 861 | SEQ ID NO: 2334 |
| KHK_miR_1862 | SEQ ID NO: 862 | SEQ ID NO: 2335 |
| KHK_miR_1863 | SEQ ID NO: 863 | SEQ ID NO: 2336 |
| KHK_miR_1864 | SEQ ID NO: 864 | SEQ ID NO: 2337 |
| KHK_miR_1865 | SEQ ID NO: 865 | SEQ ID NO: 2338 |
| KHK_miR_1866 | SEQ ID NO: 866 | SEQ ID NO: 2339 |
| KHK_miR_1867 | SEQ ID NO: 867 | SEQ ID NO: 2340 |
| KHK_miR_1868 | SEQ ID NO: 868 | SEQ ID NO: 2341 |
| KHK_miR_1869 | SEQ ID NO: 869 | SEQ ID NO: 2342 |
| KHK_miR_1870 | SEQ ID NO: 870 | SEQ ID NO: 2343 |
| | | SEQ ID NO: 2344 |
| KHK_miR_1871 | SEQ ID NO: 871 | SEQ ID NO: 2345 |
| KHK_miR_1872 | SEQ ID NO: 872 | SEQ ID NO: 2346 |
| KHK_miR_1873 | SEQ ID NO: 873 | SEQ ID NO: 2347 |
| KHK_miR_1874 | SEQ ID NO: 874 | SEQ ID NO: 2348 |
| KHK_miR_1875 | SEQ ID NO: 875 | SEQ ID NO: 2349 |
| KHK_miR_1876 | SEQ ID NO: 876 | SEQ ID NO: 2350 |

TABLE 1-27

| nucleic acid | | micro-RNA |
|---|---|---|
| name | micro-RNA | precursor |
| KHK_miR_1877 | SEQ ID NO: 877 | SEQ ID NO: 2351 |
| KHK_miR_1878 | SEQ ID NO: 878 | SEQ ID NO: 2352 |
| KHK_miR_1879 | SEQ ID NO: 879 | SEQ ID NO: 2353 |
| | | SEQ ID NO: 2354 |
| KHK_miR_1880 | SEQ ID NO: 880 | SEQ ID NO: 2355 |
| KHK_miR_1881 | SEQ ID NO: 881 | SEQ ID NO: 2356 |
| KHK_miR_1882 | SEQ ID NO: 882 | SEQ ID NO: 2357 |
| KHK_miR_1883 | SEQ ID NO: 883 | SEQ ID NO: 2358 |
| KHK_miR_1884 | SEQ ID NO: 884 | SEQ ID NO: 2359 |
| KHK_miR_1885 | SEQ ID NO: 885 | SEQ ID NO: 2360 |
| KHK_miR_1886 | SEQ ID NO: 886 | SEQ ID NO: 2361 |
| KHK_miR_1887 | SEQ ID NO: 887 | SEQ ID NO: 2362 |
| KHK_miR_1888 | SEQ ID NO: 888 | SEQ ID NO: 2363 |
| KHK_miR_1889 | SEQ ID NO: 889 | SEQ ID NO: 2364 |
| KHK_miR_1890 | SEQ ID NO: 890 | SEQ ID NO: 2365 |
| KHK_miR_1891 | SEQ ID NO: 891 | SEQ ID NO: 2366 |
| KHK_miR_1892 | SEQ ID NO: 892 | SEQ ID NO: 2367 |
| KHK_miR_1893 | SEQ ID NO: 893 | SEQ ID NO: 2368 |
| KHK_miR_1894 | SEQ ID NO: 894 | SEQ ID NO: 2369 |
| | | SEQ ID NO: 2370 |
| | | SEQ ID NO: 2371 |
| KHK_miR_1895 | SEQ ID NO: 895 | SEQ ID NO: 2372 |
| KHK_miR_1896 | SEQ ID NO: 896 | SEQ ID NO: 2373 |
| KHK_miR_1897 | SEQ ID NO: 897 | SEQ ID NO: 2374 |
| KHK_miR_1898 | SEQ ID NO: 898 | SEQ ID NO: 2375 |
| | | SEQ ID NO: 2376 |
| | | SEQ ID NO: 2377 |
| KHK_miR_1899 | SEQ ID NO: 899 | SEQ ID NO: 2378 |
| KHK_miR_1900 | SEQ ID NO: 900 | SEQ ID NO: 2379 |
| KHK_miR_1901 | SEQ ID NO: 901 | SEQ ID NO: 2380 |
| KHK_miR_1902 | SEQ ID NO: 902 | SEQ ID NO: 2381 |
| | | SEQ ID NO: 2382 |
| KHK_miR_1903 | SEQ ID NO: 903 | SEQ ID NO: 2383 |
| | | SEQ ID NO: 2384 |
| KHK_miR_1904 | SEQ ID NO: 904 | SEQ ID NO: 2385 |
| KHK_miR_1905 | SEQ ID NO: 905 | SEQ ID NO: 2386 |
| KHK_miR_1906 | SEQ ID NO: 906 | SEQ ID NO: 2387 |
| KHK_miR_1907 | SEQ ID NO: 907 | SEQ ID NO: 2388 |
| | | SEQ ID NO: 2389 |

TABLE 1-28

| nucleic acid | | micro-RNA |
|---|---|---|
| name | micro-RNA | precursor |
| KHK_miR_1908 | SEQ ID NO: 908 | SEQ ID NO: 2390 |
| KHK_miR_1909 | SEQ ID NO: 909 | SEQ ID NO: 2391 |
| KHK_miR_1910 | SEQ ID NO: 910 | SEQ ID NO: 2392 |
| KHK_miR_1911 | SEQ ID NO: 911 | SEQ ID NO: 2393 |
| KHK_miR_1912 | SEQ ID NO: 912 | SEQ ID NO: 2394 |
| KHK_miR_1913 | SEQ ID NO: 913 | SEQ ID NO: 2395 |
| KHK_miR_1914 | SEQ ID NO: 914 | SEQ ID NO: 2396 |
| KHK_miR_1915 | SEQ ID NO: 915 | SEQ ID NO: 2397 |
| KHK_miR_1916 | SEQ ID NO: 916 | SEQ ID NO: 2398 |
| KHK_miR_1917 | SEQ ID NO: 917 | SEQ ID NO: 2399 |
| KHK_miR_1918 | SEQ ID NO: 918 | SEQ ID NO: 2400 |
| KHK_miR_1919 | SEQ ID NO: 919 | SEQ ID NO: 2401 |
| KHK_miR_1920 | SEQ ID NO: 920 | SEQ ID NO: 2402 |
| | | SEQ ID NO: 2403 |
| | | SEQ ID NO: 2404 |
| | | SEQ ID NO: 2405 |
| | | SEQ ID NO: 2406 |
| | | SEQ ID NO: 2407 |
| KHK_miR_1921 | SEQ ID NO: 921 | SEQ ID NO: 2408 |
| | | SEQ ID NO: 2409 |
| | | SEQ ID NO: 2410 |
| KHK_miR_1922 | SEQ ID NO: 922 | SEQ ID NO: 2411 |
| KHK_miR_1923 | SEQ ID NO: 923 | SEQ ID NO: 2412 |
| | | SEQ ID NO: 2413 |
| KHK_miR_1924 | SEQ ID NO: 924 | SEQ ID NO: 2414 |
| KHK_miR_1925 | SEQ ID NO: 925 | SEQ ID NO: 2415 |
| | | SEQ ID NO: 2416 |
| | | SEQ ID NO: 2417 |
| KHK_miR_1926 | SEQ ID NO: 926 | SEQ ID NO: 2418 |
| KHK_miR_1927 | SEQ ID NO: 927 | SEQ ID NO: 2419 |
| KHK_miR_1928 | SEQ ID NO: 928 | SEQ ID NO: 2420 |
| KHK_miR_1929 | SEQ ID NO: 929 | SEQ ID NO: 2421 |
| | | SEQ ID NO: 2422 |
| KHK_miR_1930 | SEQ ID NO: 930 | SEQ ID NO: 2423 |
| KHK_miR_1931 | SEQ ID NO: 931 | SEQ ID NO: 2424 |
| | | SEQ ID NO: 2425 |
| | | SEQ ID NO: 2426 |
| | | SEQ ID NO: 2427 |
| KHK_miR_1932 | SEQ ID NO: 932 | SEQ ID NO: 2428 |

TABLE 1-29

| nucleic acid | | micro-RNA |
|---|---|---|
| name | micro-RNA | precursor |
| | | SEQ ID NO: 2429 |
| | | SEQ ID NO: 2430 |
| KHK_miR_1933 | SEQ ID NO: 933 | SEQ ID NO: 2431 |
| KHK_miR_1934 | SEQ ID NO: 934 | SEQ ID NO: 2432 |
| KHK_miR_1935 | SEQ ID NO: 935 | SEQ ID NO: 2433 |
| | | SEQ ID NO: 2434 |
| KHK_miR_1936 | SEQ ID NO: 936 | SEQ ID NO: 2435 |
| KHK_miR_1937 | SEQ ID NO: 937 | SEQ ID NO: 2436 |
| | | SEQ ID NO: 2437 |
| KHK_miR_1938 | SEQ ID NO: 938 | SEQ ID NO: 2438 |
| KHK_miR_1939 | SEQ ID NO: 939 | SEQ ID NO: 2439 |
| KHK_miR_1940 | SEQ ID NO: 940 | SEQ ID NO: 2440 |
| KHK_miR_1941 | SEQ ID NO: 941 | SEQ ID NO: 2441 |
| | | SEQ ID NO: 2442 |
| | | SEQ ID NO: 2443 |
| KHK_miR_1942 | SEQ ID NO: 942 | SEQ ID NO: 2444 |
| | | SEQ ID NO: 2445 |
| KHK_miR_1943 | SEQ ID NO: 943 | SEQ ID NO: 2446 |
| KHK_miR_1944 | SEQ ID NO: 944 | SEQ ID NO: 2447 |
| KHK_miR_1945 | SEQ ID NO: 945 | SEQ ID NO: 2448 |
| KHK_miR_1946 | SEQ ID NO: 946 | SEQ ID NO: 2449 |
| KHK_miR_1947 | SEQ ID NO: 947 | SEQ ID NO: 2450 |
| KHK_miR_1948 | SEQ ID NO: 948 | SEQ ID NO: 2451 |
| | | SEQ ID NO: 2452 |
| KHK_miR_1949 | SEQ ID NO: 949 | SEQ ID NO: 2453 |
| | | SEQ ID NO: 2454 |
| KHK_miR_1950 | SEQ ID NO: 950 | SEQ ID NO: 2455 |
| KHK_miR_1951 | SEQ ID NO: 951 | SEQ ID NO: 2456 |
| KHK_miR_1952 | SEQ ID NO: 952 | SEQ ID NO: 2457 |
| KHK_miR_1953 | SEQ ID NO: 953 | SEQ ID NO: 2458 |
| | | SEQ ID NO: 2459 |
| | | SEQ ID NO: 2460 |
| KHK_miR_1954 | SEQ ID NO: 954 | SEQ ID NO: 2461 |
| | | SEQ ID NO: 2462 |
| KHK_miR_1955 | SEQ ID NO: 955 | SEQ ID NO: 2463 |
| KHK_miR_1956 | SEQ ID NO: 956 | SEQ ID NO: 2464 |
| KHK_miR_1957 | SEQ ID NO: 957 | SEQ ID NO: 2465 |
| KHK_miR_1958 | SEQ ID NO: 958 | SEQ ID NO: 2466 |
| KHK_miR_1959 | SEQ ID NO: 959 | SEQ ID NO: 2467 |

TABLE 1-30

| nucleic acid | | micro-RNA |
|---|---|---|
| name | micro-RNA | precursor |
| KHK_miR_1960 | SEQ ID NO: 960 | SEQ ID NO: 2468 |
| KHK_miR_1961 | SEQ ID NO: 961 | SEQ ID NO: 2469 |
| KHK_miR_1962 | SEQ ID NO: 962 | SEQ ID NO: 2470 |
| KHK_miR_1963 | SEQ ID NO: 963 | SEQ ID NO: 2471 |
| | | SEQ ID NO: 2472 |
| | | SEQ ID NO: 2473 |
| KHK_miR_1964 | SEQ ID NO: 964 | SEQ ID NO: 2474 |
| KHK_miR_1965 | SEQ ID NO: 965 | SEQ ID NO: 2475 |
| KHK_miR_1966 | SEQ ID NO: 966 | SEQ ID NO: 2476 |
| KHK_miR_1967 | SEQ ID NO: 967 | SEQ ID NO: 2477 |
| | | SEQ ID NO: 2478 |
| KHK_miR_1968 | SEQ ID NO: 968 | SEQ ID NO: 2479 |
| KHK_miR_1969 | SEQ ID NO: 969 | SEQ ID NO: 2480 |
| KHK_miR_1970 | SEQ ID NO: 970 | SEQ ID NO: 2481 |
| KHK_miR_1971 | SEQ ID NO: 971 | SEQ ID NO: 2482 |
| KHK_miR_1972 | SEQ ID NO: 972 | SEQ ID NO: 2483 |
| KHK_miR_1973 | SEQ ID NO: 973 | SEQ ID NO: 2484 |
| KHK_miR_1974 | SEQ ID NO: 974 | SEQ ID NO: 2485 |
| KHK_miR_1975 | SEQ ID NO: 975 | SEQ ID NO: 2486 |
| KHK_miR_1976 | SEQ ID NO: 976 | SEQ ID NO: 2487 |
| KHK_miR_1977 | SEQ ID NO: 977 | SEQ ID NO: 2488 |
| KHK_miR_1978 | SEQ ID NO: 978 | SEQ ID NO: 2489 |
| KHK_miR_1979 | SEQ ID NO: 979 | SEQ ID NO: 2490 |
| KHK_miR_1980 | SEQ ID NO: 980 | SEQ ID NO: 2491 |
| KHK_miR_1981 | SEQ ID NO: 981 | SEQ ID NO: 2492 |
| KHK_miR_1982 | SEQ ID NO: 982 | SEQ ID NO: 2493 |
| KHK_miR_1983 | SEQ ID NO: 983 | SEQ ID NO: 2494 |
| KHK_miR_1984 | SEQ ID NO: 984 | SEQ ID NO: 2495 |
| KHK_miR_1985 | SEQ ID NO: 985 | SEQ ID NO: 2496 |
| KHK_miR_1986 | SEQ ID NO: 986 | SEQ ID NO: 2497 |
| KHK_miR_1987 | SEQ ID NO: 987 | SEQ ID NO: 2498 |
| KHK_miR_1988 | SEQ ID NO: 988 | SEQ ID NO: 2499 |
| KHK_miR_1989 | SEQ ID NO: 989 | SEQ ID NO: 2500 |
| KHK_miR_1990 | SEQ ID NO: 990 | SEQ ID NO: 2501 |
| KHK_miR_1991 | SEQ ID NO: 991 | SEQ ID NO: 2502 |
| KHK_miR_1992 | SEQ ID NO: 992 | SEQ ID NO: 2503 |
| KHK_miR_1993 | SEQ ID NO: 993 | SEQ ID NO: 2504 |
| KHK_miR_1994 | SEQ ID NO: 994 | SEQ ID NO: 2505 |
| KHK_miR_1995 | SEQ ID NO: 995 | SEQ ID NO: 2506 |

TABLE 1-31

| nucleic acid | | micro-RNA |
|---|---|---|
| name | micro-RNA | precursor |
| KHK_miR_1996 | SEQ ID NO: 996 | SEQ ID NO: 2507 |
| KHK_miR_1997 | SEQ ID NO: 997 | SEQ ID NO: 2508 |
| KHK_miR_1998 | SEQ ID NO: 998 | SEQ ID NO: 2509 |
| KHK_miR_1999 | SEQ ID NO: 999 | SEQ ID NO: 2510 |
| KHK_miR_2000 | SEQ ID NO: 1000 | SEQ ID NO: 2511 |
| KHK_miR_2001 | SEQ ID NO: 1001 | SEQ ID NO: 2512 |
| | | SEQ ID NO: 2513 |
| KHK_miR_2002 | SEQ ID NO: 1002 | SEQ ID NO: 2514 |
| KHK_miR_2003 | SEQ ID NO: 1003 | SEQ ID NO: 2515 |
| KHK_miR_2004 | SEQ ID NO: 1004 | SEQ ID NO: 2516 |
| KHK_miR_2005 | SEQ ID NO: 1005 | SEQ ID NO: 2517 |
| KHK_miR_2006 | SEQ ID NO: 1006 | SEQ ID NO: 2518 |
| KHK_miR_2007 | SEQ ID NO: 1007 | SEQ ID NO: 2519 |
| KHK_miR_2008 | SEQ ID NO: 1008 | SEQ ID NO: 2520 |
| KHK_miR_2009 | SEQ ID NO: 1009 | SEQ ID NO: 2521 |
| KHK_miR_2010 | SEQ ID NO: 1010 | SEQ ID NO: 2522 |
| KHK_miR_2011 | SEQ ID NO: 1011 | SEQ ID NO: 2523 |
| KHK_miR_2012 | SEQ ID NO: 1012 | SEQ ID NO: 2524 |
| KHK_miR_2013 | SEQ ID NO: 1013 | SEQ ID NO: 2525 |
| KHK_miR_2014 | SEQ ID NO: 1014 | SEQ ID NO: 2526 |
| KHK_miR_2015 | SEQ ID NO: 1015 | SEQ ID NO: 2527 |
| KHK_miR_2016 | SEQ ID NO: 1016 | SEQ ID NO: 2528 |
| KHK_miR_2017 | SEQ ID NO: 1017 | SEQ ID NO: 2529 |
| | | SEQ ID NO: 2530 |
| | | SEQ ID NO: 2531 |
| KHK_miR_2018 | SEQ ID NO: 1018 | SEQ ID NO: 2532 |
| KHK_miR_2019 | SEQ ID NO: 1019 | SEQ ID NO: 2533 |
| KHK_miR_2020 | SEQ ID NO: 1020 | SEQ ID NO: 2534 |
| KHK_miR_2021 | SEQ ID NO: 1021 | SEQ ID NO: 2535 |
| KHK_miR_2022 | SEQ ID NO: 1022 | SEQ ID NO: 2536 |
| KHK_miR_2023 | SEQ ID NO: 1023 | SEQ ID NO: 2537 |
| KHK_miR_2024 | SEQ ID NO: 1024 | SEQ ID NO: 2538 |
| KHK_miR_2025 | SEQ ID NO: 1025 | SEQ ID NO: 2539 |
| | | SEQ ID NO: 2540 |
| | | SEQ ID NO: 2541 |
| | | SEQ ID NO: 2542 |
| KHK_miR_2026 | SEQ ID NO: 1026 | SEQ ID NO: 2543 |
| KHK_miR_2027 | SEQ ID NO: 1027 | SEQ ID NO: 2544 |
| KHK_miR_2028 | SEQ ID NO: 1028 | SEQ ID NO: 2545 |

TABLE 1-32

| nucleic acid | | micro-RNA |
|---|---|---|
| name | micro-RNA | precursor |
| KHK_miR_2029 | SEQ ID NO: 1029 | SEQ ID NO: 2546 |
| KHK_miR_2030 | SEQ ID NO: 1030 | SEQ ID NO: 2547 |
| KHK_miR_2031 | SEQ ID NO: 1031 | SEQ ID NO: 2548 |
| KHK_miR_2032 | SEQ ID NO: 1032 | SEQ ID NO: 2549 |
| KHK_miR_2033 | SEQ ID NO: 1033 | SEQ ID NO: 2550 |
| KHK_miR_2034 | SEQ ID NO: 1034 | SEQ ID NO: 2551 |
| KHK_miR_2035 | SEQ ID NO: 1035 | SEQ ID NO: 2552 |
| KHK_miR_2036 | SEQ ID NO: 1036 | SEQ ID NO: 2553 |
| KHK_miR_2037 | SEQ ID NO: 1037 | SEQ ID NO: 2554 |
| | | SEQ ID NO: 2555 |
| | | SEQ ID NO: 2556 |
| | | SEQ ID NO: 2557 |
| KHK_miR_2038 | SEQ ID NO: 1038 | SEQ ID NO: 2558 |
| KHK_miR_2039 | SEQ ID NO: 1039 | SEQ ID NO: 2559 |
| KHK_miR_2040 | SEQ ID NO: 1040 | SEQ ID NO: 2560 |
| KHK_miR_2041 | SEQ ID NO: 1041 | SEQ ID NO: 2561 |
| KHK_miR_2042 | SEQ ID NO: 1042 | SEQ ID NO: 2562 |
| KHK_miR_2043 | SEQ ID NO: 1043 | SEQ ID NO: 2563 |
| KHK_miR_2044 | SEQ ID NO: 1044 | SEQ ID NO: 2564 |
| KHK_miR_2045 | SEQ ID NO: 1045 | SEQ ID NO: 2565 |
| KHK_miR_2046 | SEQ ID NO: 1046 | SEQ ID NO: 2566 |
| KHK_miR_2047 | SEQ ID NO: 1047 | SEQ ID NO: 2567 |
| KHK_miR_2048 | SEQ ID NO: 1048 | SEQ ID NO: 2568 |
| KHK_miR_2049 | SEQ ID NO: 1049 | SEQ ID NO: 2569 |
| | | SEQ ID NO: 2570 |
| KHK_miR_2050 | SEQ ID NO: 1050 | SEQ ID NO: 2571 |
| KHK_miR_2051 | SEQ ID NO: 1051 | SEQ ID NO: 2572 |
| KHK_miR_2052 | SEQ ID NO: 1052 | SEQ ID NO: 2573 |
| KHK_miR_2053 | SEQ ID NO: 1053 | SEQ ID NO: 2574 |
| KHK_miR_2054 | SEQ ID NO: 1054 | SEQ ID NO: 2575 |
| KHK_miR_2055 | SEQ ID NO: 1055 | SEQ ID NO: 2576 |
| KHK_miR_2056 | SEQ ID NO: 1056 | SEQ ID NO: 2577 |
| KHK_miR_2057 | SEQ ID NO: 1057 | SEQ ID NO: 2578 |
| KHK_miR_2058 | SEQ ID NO: 1058 | SEQ ID NO: 2579 |
| KHK_miR_2059 | SEQ ID NO: 1059 | SEQ ID NO: 2580 |
| KHK_miR_2060 | SEQ ID NO: 1060 | SEQ ID NO: 2581 |
| KHK_miR_2061 | SEQ ID NO: 1061 | SEQ ID NO: 2582 |
| KHK_miR_2062 | SEQ ID NO: 1062 | SEQ ID NO: 2583 |
| KHK_miR_2063 | SEQ ID NO: 1063 | SEQ ID NO: 2584 |

TABLE 1-33

| nucleic acid | | micro-RNA |
|---|---|---|
| name | micro-RNA | precursor |
| KHK_miR_2064 | SEQ ID NO: 1064 | SEQ ID NO: 2585 |
| KHK_miR_2065 | SEQ ID NO: 1065 | SEQ ID NO: 2586 |
| KHK_miR_2066 | SEQ ID NO: 1066 | SEQ ID NO: 2587 |
| KHK_miR_2067 | SEQ ID NO: 1067 | SEQ ID NO: 2588 |
| KHK_miR_2068 | SEQ ID NO: 1068 | SEQ ID NO: 2589 |
| KHK_miR_2069 | SEQ ID NO: 1069 | SEQ ID NO: 2590 |
| KHK_miR_2070 | SEQ ID NO: 1070 | SEQ ID NO: 2591 |
| KHK_miR_2071 | SEQ ID NO: 1071 | SEQ ID NO: 2592 |
| KHK_miR_2072 | SEQ ID NO: 1072 | SEQ ID NO: 2593 |
| KHK_miR_2073 | SEQ ID NO: 1073 | SEQ ID NO: 2594 |
| | | SEQ ID NO: 2595 |
| KHK_miR_2074 | SEQ ID NO: 1074 | SEQ ID NO: 2596 |
| KHK_miR_2075 | SEQ ID NO: 1075 | SEQ ID NO: 2597 |
| KHK_miR_2076 | SEQ ID NO: 1076 | SEQ ID NO: 2598 |
| KHK_miR_2077 | SEQ ID NO: 1077 | SEQ ID NO: 2599 |
| KHK_miR_2078 | SEQ ID NO: 1078 | SEQ ID NO: 2600 |
| KHK_miR_2079 | SEQ ID NO: 1079 | SEQ ID NO: 2601 |
| KHK_miR_2080 | SEQ ID NO: 1080 | SEQ ID NO: 2602 |
| KHK_miR_2081 | SEQ ID NO: 1081 | SEQ ID NO: 2603 |
| KHK_miR_2082 | SEQ ID NO: 1082 | SEQ ID NO: 2604 |
| KHK_miR_2083 | SEQ ID NO: 1083 | SEQ ID NO: 2605 |
| | | SEQ ID NO: 2606 |
| KHK_miR_2084 | SEQ ID NO: 1084 | SEQ ID NO: 2607 |
| KHK_miR_2085 | SEQ ID NO: 1085 | SEQ ID NO: 2608 |
| KHK_miR_2086 | SEQ ID NO: 1086 | SEQ ID NO: 2609 |
| KHK_miR_2087 | SEQ ID NO: 1087 | SEQ ID NO: 2610 |
| KHK_miR_2088 | SEQ ID NO: 1088 | SEQ ID NO: 2611 |
| KHK_miR_2089 | SEQ ID NO: 1089 | SEQ ID NO: 2612 |
| KHK_miR_2090 | SEQ ID NO: 1090 | SEQ ID NO: 2613 |
| KHK_miR_2091 | SEQ ID NO: 1091 | SEQ ID NO: 2614 |
| KHK_miR_2092 | SEQ ID NO: 1092 | SEQ ID NO: 2615 |
| KHK_miR_2093 | SEQ ID NO: 1093 | SEQ ID NO: 2616 |
| KHK_miR_2094 | SEQ ID NO: 1094 | SEQ ID NO: 2617 |
| KHK_miR_2095 | SEQ ID NO: 1095 | SEQ ID NO: 2618 |
| KHK_miR_2096 | SEQ ID NO: 1096 | SEQ ID NO: 2619 |
| KHK_miR_2097 | SEQ ID NO: 1097 | SEQ ID NO: 2620 |
| | | SEQ ID NO: 2621 |
| KHK_miR_2098 | SEQ ID NO: 1098 | SEQ ID NO: 2622 |
| | | SEQ ID NO: 2623 |

TABLE 1-34

| nucleic acid | | micro-RNA |
|---|---|---|
| name | micro-RNA | precursor |
| | | SEQ ID NO: 2624 |
| KHK_miR_2099 | SEQ ID NO: 1099 | SEQ ID NO: 2625 |
| KHK_miR_2100 | SEQ ID NO: 1100 | SEQ ID NO: 2626 |
| | | SEQ ID NO: 2627 |
| | | SEQ ID NO: 2628 |
| | | SEQ ID NO: 2629 |
| | | SEQ ID NO: 2630 |
| KHK_miR_2101 | SEQ ID NO: 1101 | SEQ ID NO: 2631 |
| KHK_miR_2102 | SEQ ID NO: 1102 | SEQ ID NO: 2632 |
| KHK_miR_2103 | SEQ ID NO: 1103 | SEQ ID NO: 2633 |
| KHK_miR_2104 | SEQ ID NO: 1104 | SEQ ID NO: 2634 |
| KHK_miR_2105 | SEQ ID NO: 1105 | SEQ ID NO: 2635 |
| KHK_miR_2106 | SEQ ID NO: 1106 | SEQ ID NO: 2636 |
| KHK_miR_2107 | SEQ ID NO: 1107 | SEQ ID NO: 2637 |
| KHK_miR_2108 | SEQ ID NO: 1108 | SEQ ID NO: 2638 |
| KHK_miR_2109 | SEQ ID NO: 1109 | SEQ ID NO: 2639 |
| KHK_miR_2110 | SEQ ID NO: 1110 | SEQ ID NO: 2640 |
| KHK_miR_2111 | SEQ ID NO: 1111 | SEQ ID NO: 2641 |
| KHK_miR_2112 | SEQ ID NO: 1112 | SEQ ID NO: 2642 |
| KHK_miR_2113 | SEQ ID NO: 1113 | SEQ ID NO: 2643 |
| KHK_miR_2114 | SEQ ID NO: 1114 | SEQ ID NO: 2644 |
| KHK_miR_2115 | SEQ ID NO: 1115 | SEQ ID NO: 2645 |
| KHK_miR_2116 | SEQ ID NO: 1116 | SEQ ID NO: 2646 |
| KHK_miR_2117 | SEQ ID NO: 1117 | SEQ ID NO: 2647 |
| KHK_miR_2118 | SEQ ID NO: 1118 | SEQ ID NO: 2648 |
| KHK_miR_2119 | SEQ ID NO: 1119 | SEQ ID NO: 2649 |
| | | SEQ ID NO: 2650 |
| KHK_miR_2120 | SEQ ID NO: 1120 | SEQ ID NO: 2651 |
| KHK_miR_2121 | SEQ ID NO: 1121 | SEQ ID NO: 2652 |
| KHK_miR_2122 | SEQ ID NO: 1122 | SEQ ID NO: 2653 |
| KHK_miR_2123 | SEQ ID NO: 1123 | SEQ ID NO: 2654 |
| KHK_miR_2124 | SEQ ID NO: 1124 | SEQ ID NO: 2655 |
| KHK_miR_2125 | SEQ ID NO: 1125 | SEQ ID NO: 2656 |
| KHK_miR_2126 | SEQ ID NO: 1126 | SEQ ID NO: 2657 |
| KHK_miR_2127 | SEQ ID NO: 1127 | SEQ ID NO: 2658 |
| KHK_miR_2128 | SEQ ID NO: 1128 | SEQ ID NO: 2659 |
| KHK_miR_2129 | SEQ ID NO: 1129 | SEQ ID NO: 2660 |
| KHK_miR_2130 | SEQ ID NO: 1130 | SEQ ID NO: 2661 |
| KHK_miR_2131 | SEQ ID NO: 1131 | SEQ ID NO: 2662 |

TABLE 1-35

| nucleic acid | | micro-RNA |
|---|---|---|
| name | micro-RNA | precursor |
| KHK_miR_2132 | SEQ ID NO: 1132 | SEQ ID NO: 2663 |
| KHK_miR_2133 | SEQ ID NO: 1133 | SEQ ID NO: 2664 |
| KHK_miR_2134 | SEQ ID NO: 1134 | SEQ ID NO: 2665 |
| KHK_miR_2135 | SEQ ID NO: 1135 | SEQ ID NO: 2666 |
| KHK_miR_2136 | SEQ ID NO: 1136 | SEQ ID NO: 2667 |
| KHK_miR_2137 | SEQ ID NO: 1137 | SEQ ID NO: 2668 |
| KHK_miR_2138 | SEQ ID NO: 1138 | SEQ ID NO: 2669 |
| KHK_miR_2139 | SEQ ID NO: 1139 | SEQ ID NO: 2670 |
| KHK_miR_2140 | SEQ ID NO: 1140 | SEQ ID NO: 2671 |
| KHK_miR_2141 | SEQ ID NO: 1141 | SEQ ID NO: 2672 |
| KHK_miR_2142 | SEQ ID NO: 1142 | SEQ ID NO: 2673 |
| KHK_miR_2143 | SEQ ID NO: 1143 | SEQ ID NO: 2674 |
| KHK_miR_2144 | SEQ ID NO: 1144 | SEQ ID NO: 2675 |
| KHK_miR_2145 | SEQ ID NO: 1145 | SEQ ID NO: 2676 |
| KHK_miR_2146 | SEQ ID NO: 1146 | SEQ ID NO: 2677 |
| KHK_miR_2147 | SEQ ID NO: 1147 | SEQ ID NO: 2678 |
| KHK_miR_2148 | SEQ ID NO: 1148 | SEQ ID NO: 2679 |
| KHK_miR_2149 | SEQ ID NO: 1149 | SEQ ID NO: 2680 |
| KHK_miR_2150 | SEQ ID NO: 1150 | SEQ ID NO: 2681 |
| KHK_miR_2151 | SEQ ID NO: 1151 | SEQ ID NO: 2682 |
| KHK_miR_2152 | SEQ ID NO: 1152 | SEQ ID NO: 2683 |
| KHK_miR_2153 | SEQ ID NO: 1153 | SEQ ID NO: 2684 |
| KHK_miR_2154 | SEQ ID NO: 1154 | SEQ ID NO: 2685 |
| KHK_miR_2155 | SEQ ID NO: 1155 | SEQ ID NO: 2686 |
| KHK_miR_2156 | SEQ ID NO: 1156 | SEQ ID NO: 2687 |
| KHK_miR_2157 | SEQ ID NO: 1157 | SEQ ID NO: 2688 |
| KHK_miR_2158 | SEQ ID NO: 1159 | SEQ ID NO: 2689 |
| KHK_miR_2159 | SEQ ID NO: 1159 | SEQ ID NO: 2690 |
| KHK_miR_2160 | SEQ ID NO: 1160 | SEQ ID NO: 2691 |
| KHK_miR_2161 | SEQ ID NO: 1161 | SEQ ID NO: 2692 |
| KHK_miR_2162 | SEQ ID NO: 1162 | SEQ ID NO: 2693 |
| KHK_miR_2163 | SEQ ID NO: 1163 | SEQ ID NO: 2694 |
| KHK_miR_2164 | SEQ ID NO: 1164 | SEQ ID NO: 2695 |
| KHK_miR_2165 | SEQ ID NO: 1165 | SEQ ID NO: 2696 |
| KHK_miR_2166 | SEQ ID NO: 1166 | SEQ ID NO: 2697 |
| KHK_miR_2167 | SEQ ID NO: 1167 | SEQ ID NO: 2698 |
| KHK_miR_2168 | SEQ ID NO: 1168 | SEQ ID NO: 2699 |
| KHK_miR_2169 | SEQ ID NO: 1169 | SEQ ID NO: 2700 |
| | | SEQ ID NO: 2701 |

TABLE 1-36

| nucleic acid | | micro-RNA |
|---|---|---|
| name | micro-RNA | precursor |
| KHK_miR_2170 | SEQ ID NO: 1170 | SEQ ID NO: 2702 |
| KHK_miR_2171 | SEQ ID NO: 1171 | SEQ ID NO: 2703 |
| KHK_miR_2172 | SEQ ID NO: 1172 | SEQ ID NO: 2704 |
| KHK_miR_2173 | SEQ ID NO: 1173 | SEQ ID NO: 2705 |
| | | SEQ ID NO: 2706 |
| KHK_miR_2174 | SEQ ID NO: 1174 | SEQ ID NO: 2707 |
| KHK_miR_2175 | SEQ ID NO: 1175 | SEQ ID NO: 2708 |
| | | SEQ ID NO: 2709 |
| | | SEQ ID NO: 2710 |
| KHK_miR_2176 | SEQ ID NO: 1176 | SEQ ID NO: 2711 |
| KHK_miR_2177 | SEQ ID NO: 1177 | SEQ ID NO: 2712 |
| | | SEQ ID NO: 2713 |
| | | SEQ ID NO: 2714 |
| KHK_miR_2178 | SEQ ID NO: 1178 | SEQ ID NO: 2715 |
| KHK_miR_2179 | SEQ ID NO: 1179 | SEQ ID NO: 2716 |
| KHK_miR_2180 | SEQ ID NO: 1180 | SEQ ID NO: 2717 |
| KHK_miR_2181 | SEQ ID NO: 1181 | SEQ ID NO: 2718 |
| KHK_miR_2182 | SEQ ID NO: 1182 | SEQ ID NO: 2719 |
| KHK_miR_2183 | SEQ ID NO: 1183 | SEQ ID NO: 2720 |
| KHK_miR_2184 | SEQ ID NO: 1184 | SEQ ID NO: 2721 |
| KHK_miR_2185 | SEQ ID NO: 1185 | SEQ ID NO: 2722 |
| KHK_miR_2186 | SEQ ID NO: 1186 | SEQ ID NO: 2723 |
| KHK_miR_2187 | SEQ ID NO: 1187 | SEQ ID NO: 2724 |
| KHK_miR_2188 | SEQ ID NO: 1188 | SEQ ID NO: 2725 |
| KHK_miR_2189 | SEQ ID NO: 1189 | SEQ ID NO: 2726 |
| KHK_miR_2190 | SEQ ID NO: 1190 | SEQ ID NO: 2727 |
| KHK_miR_2191 | SEQ ID NO: 1191 | SEQ ID NO: 2728 |
| KHK_miR_2192 | SEQ ID NO: 1192 | SEQ ID NO: 2729 |
| KHK_miR_2193 | SEQ ID NO: 1193 | SEQ ID NO: 2730 |
| KHK_miR_2194 | SEQ ID NO: 1194 | SEQ ID NO: 2731 |
| KHK_miR_2195 | SEQ ID NO: 1195 | SEQ ID NO: 2732 |
| KHK_miR_2196 | SEQ ID NO: 1196 | SEQ ID NO: 2733 |
| KHK_miR_2197 | SEQ ID NO: 1197 | SEQ ID NO: 2734 |
| KHK_miR_2198 | SEQ ID NO: 1198 | SEQ ID NO: 2735 |
| KHK_miR_2199 | SEQ ID NO: 1199 | SEQ ID NO: 2736 |
| KHK_miR_2200 | SEQ ID NO: 1200 | SEQ ID NO: 2737 |
| KHK_miR_2201 | SEQ ID NO: 1201 | SEQ ID NO: 2738 |
| KHK_miR_2202 | SEQ ID NO: 1202 | SEQ ID NO: 2739 |
| KHK_miR_2203 | SEQ ID NO: 1203 | SEQ ID NO: 2740 |

TABLE 1-37

| nucleic acid | | micro-RNA |
|---|---|---|
| name | micro-RNA | precursor |
| | | SEQ ID NO: 2741 |
| | | SEQ ID NO: 2742 |
| KHK_miR_2204 | SEQ ID NO: 1204 | SEQ ID NO: 2743 |
| KHK_miR_2205 | SEQ ID NO: 1205 | SEQ ID NO: 2744 |
| KHK_miR_2206 | SEQ ID NO: 1206 | SEQ ID NO: 2745 |
| | | SEQ ID NO: 2746 |
| KHK_miR_2207 | SEQ ID NO: 1207 | SEQ ID NO: 2747 |
| KHK_miR_2208 | SEQ ID NO: 1208 | SEQ ID NO: 2748 |
| KHK_miR_2209 | SEQ ID NO: 1209 | SEQ ID NO: 2749 |
| KHK_miR_2210 | SEQ ID NO: 1210 | SEQ ID NO: 2750 |
| KHK_miR_2211 | SEQ ID NO: 1211 | SEQ ID NO: 2751 |
| KHK_miR_2212 | SEQ ID NO: 1212 | SEQ ID NO: 2752 |
| KHK_miR_2213 | SEQ ID NO: 1213 | SEQ ID NO: 2753 |
| KHK_miR_2214 | SEQ ID NO: 1214 | SEQ ID NO: 2754 |
| KHK_miR_2215 | SEQ ID NO: 1215 | SEQ ID NO: 2755 |
| KHK_miR_2216 | SEQ ID NO: 1216 | SEQ ID NO: 2756 |
| KHK_miR_2217 | SEQ ID NO: 1217 | SEQ ID NO: 2757 |
| KHK_miR_2218 | SEQ ID NO: 1218 | SEQ ID NO: 2758 |
| KHK_miR_2219 | SEQ ID NO: 1219 | SEQ ID NO: 2759 |
| | | SEQ ID NO: 2760 |
| KHK_miR_2220 | SEQ ID NO: 1220 | SEQ ID NO: 2761 |
| KHK_miR_2221 | SEQ ID NO: 1221 | SEQ ID NO: 2762 |
| KHK_miR_2222 | SEQ ID NO: 1222 | SEQ ID NO: 2763 |
| | | SEQ ID NO: 2764 |
| KHK_miR_2223 | SEQ ID NO: 1223 | SEQ ID NO: 2765 |
| KHK_miR_2224 | SEQ ID NO: 1224 | SEQ ID NO: 2766 |
| KHK_miR_2225 | SEQ ID NO: 1225 | SEQ ID NO: 2767 |
| KHK_miR_2226 | SEQ ID NO: 1226 | SEQ ID NO: 2768 |
| KHK_miR_2227 | SEQ ID NO: 1227 | SEQ ID NO: 2769 |
| KHK_miR_2228 | SEQ ID NO: 1228 | SEQ ID NO: 2770 |
| KHK_miR_2229 | SEQ ID NO: 1229 | SEQ ID NO: 2771 |
| KHK_miR_2230 | SEQ ID NO: 1230 | SEQ ID NO: 2772 |
| KHK_miR_2231 | SEQ ID NO: 1231 | SEQ ID NO: 2773 |
| KHK_miR_2232 | SEQ ID NO: 1232 | SEQ ID NO: 2774 |
| | | SEQ ID NO: 2775 |
| | | SEQ ID NO: 2776 |
| KHK_miR_2233 | SEQ ID NO: 1233 | SEQ ID NO: 2777 |
| KHK_miR_2234 | SEQ ID NO: 1234 | SEQ ID NO: 2778 |
| | | SEQ ID NO: 2779 |

TABLE 1-38

| nucleic acid | | micro-RNA |
|---|---|---|
| name | micro-RNA | precursor |
| | | SEQ ID NO: 2780 |
| | | SEQ ID NO: 2781 |
| | | SEQ ID NO: 2782 |
| KHK_miR_2235 | SEQ ID NO: 1235 | SEQ ID NO: 2783 |
| KHK_miR_2236 | SEQ ID NO: 1236 | SEQ ID NO: 2784 |
| KHK_miR_2237 | SEQ ID NO: 1237 | SEQ ID NO: 2785 |
| KHK_miR_2238 | SEQ ID NO: 1238 | SEQ ID NO: 2786 |
| | | SEQ ID NO: 2787 |
| | | SEQ ID NO: 2788 |
| KHK_miR_2239 | SEQ ID NO: 1239 | SEQ ID NO: 2789 |
| KHK_miR_2240 | SEQ ID NO: 1240 | SEQ ID NO: 2790 |
| KHK_miR_2241 | SEQ ID NO: 1241 | SEQ ID NO: 2791 |
| KHK_miR_2242 | SEQ ID NO: 1242 | SEQ ID NO: 2792 |
| KHK_miR_2243 | SEQ ID NO: 1243 | SEQ ID NO: 2793 |
| KHK_miR_2244 | SEQ ID NO: 1244 | SEQ ID NO: 2794 |
| KHK_miR_2245 | SEQ ID NO: 1245 | SEQ ID NO: 2795 |
| KHK_miR_2246 | SEQ ID NO: 1246 | SEQ ID NO: 2796 |
| KHK_miR_2247 | SEQ ID NO: 1247 | SEQ ID NO: 2797 |
| KHK_miR_2248 | SEQ ID NO: 1248 | SEQ ID NO: 2798 |
| KHK_miR_2249 | SEQ ID NO: 1249 | SEQ ID NO: 2799 |
| KHK_miR_2250 | SEQ ID NO: 1250 | SEQ ID NO: 2800 |
| KHK_miR_2251 | SEQ ID NO: 1251 | SEQ ID NO: 2801 |
| KHK_miR_2252 | SEQ ID NO: 1252 | SEQ ID NO: 2802 |
| | | SEQ ID NO: 2803 |
| | | SEQ ID NO: 2804 |
| | | SEQ ID NO: 2805 |
| KHK_miR_2253 | SEQ ID NO: 1253 | SEQ ID NO: 2806 |
| KHK_miR_2254 | SEQ ID NO: 1254 | SEQ ID NO: 2807 |
| KHK_miR_2255 | SEQ ID NO: 1255 | SEQ ID NO: 2808 |
| KHK_miR_2256 | SEQ ID NO: 1256 | SEQ ID NO: 2809 |
| KHK_miR_2257 | SEQ ID NO: 1257 | SEQ ID NO: 2810 |
| KHK_miR_2258 | SEQ ID NO: 1258 | SEQ ID NO: 2811 |
| KHK_miR_2259 | SEQ ID NO: 1259 | SEQ ID NO: 2812 |
| | | SEQ ID NO: 2813 |
| KHK_miR_2260 | SEQ ID NO: 1260 | SEQ ID NO: 2814 |
| KHK_miR_2261 | SEQ ID NO: 1261 | SEQ ID NO: 2815 |
| KHK_miR_2262 | SEQ ID NO: 1262 | SEQ ID NO: 2816 |
| | | SEQ ID NO: 2817 |
| | | SEQ ID NO: 2818 |

TABLE 1-39

| nucleic acid | | micro-RNA |
|---|---|---|
| name | micro-RNA | precursor |
| | | SEQ ID NO: 2819 |
| | | SEQ ID NO: 2820 |
| KHK_miR_2263 | SEQ ID NO: 1263 | SEQ ID NO: 2821 |
| KHK_miR_2264 | SEQ ID NO: 1264 | SEQ ID NO: 2822 |
| KHK_miR_2265 | SEQ ID NO: 1265 | SEQ ID NO: 2823 |
| KHK_miR_2267 | SEQ ID NO: 1266 | SEQ ID NO: 2824 |
| KHK_miR_2269 | SEQ ID NO: 1267 | SEQ ID NO: 2825 |
| KHK_miR_2271 | SEQ ID NO: 1268 | SEQ ID NO: 2826 |
| KHK_miR_2272 | SEQ ID NO: 1269 | SEQ ID NO: 2827 |
| KHK_miR_2273 | SEQ ID NO: 1270 | SEQ ID NO: 2828 |
| KHK_miR_2274 | SEQ ID NO: 1271 | SEQ ID NO: 2829 |
| KHK_miR_2278 | SEQ ID NO: 1272 | SEQ ID NO: 2830 |
| KHK_miR_2280 | SEQ ID NO: 1273 | SEQ ID NO: 2831 |
| KHK_miR_2281 | SEQ ID NO: 1274 | SEQ ID NO: 2832 |
| KHK_miR_2282 | SEQ ID NO: 1275 | SEQ ID NO: 2833 |
| KHK_miR_2283 | SEQ ID NO: 1276 | SEQ ID NO: 2834 |
| KHK_miR_2284 | SEQ ID NO: 1277 | SEQ ID NO: 2835 |
| KHK_miR_2285 | SEQ ID NO: 1278 | SEQ ID NO: 2836 |
| KHK_miR_2286 | SEQ ID NO: 1279 | SEQ ID NO: 2837 |
| KHK_miR_2287 | SEQ ID NO: 1280 | SEQ ID NO: 2838 |
| KHK_miR_2288 | SEQ ID NO: 1281 | SEQ ID NO: 2839 |
| KHK_miR_2289 | SEQ ID NO: 1282 | SEQ ID NO: 2840 |
| KHK_miR_2290 | SEQ ID NO: 1283 | SEQ ID NO: 2841 |
| KHK_miR_2291 | SEQ ID NO: 1284 | SEQ ID NO: 2842 |
| KHK_miR_2292 | SEQ ID NO: 1285 | SEQ ID NO: 2843 |
| KHK_miR_2293 | SEQ ID NO: 1286 | SEQ ID NO: 2844 |
| KHK_miR_2294 | SEQ ID NO: 1287 | SEQ ID NO: 2845 |
| KHK_miR_2295 | SEQ ID NO: 1288 | SEQ ID NO: 2846 |
| KHK_miR_2296 | SEQ ID NO: 1289 | SEQ ID NO: 2847 |
| KHK_miR_2297 | SEQ ID NO: 1290 | SEQ ID NO: 2848 |
| KHK_miR_2298 | SEQ ID NO: 1291 | SEQ ID NO: 2849 |
| KHK_miR_2299 | SEQ ID NO: 1292 | SEQ ID NO: 2850 |
| KHK_miR_2300 | SEQ ID NO: 1293 | SEQ ID NO: 2851 |
| KHK_miR_2301 | SEQ ID NO: 1294 | pre-hsa-let-7a-1 |
| KHK_miR_2302 | SEQ ID NO: 1295 | pre-hsa-let-7d |
| KHK_miR_2303 | SEQ ID NO: 1296 | pre-hsa-let-7f-2 |
| KHK_miR_2304 | SEQ ID NO: 1297 | pre-hsa-mir-101-1 |
| KHK_miR_2305 | SEQ ID NO: 1298 | pre-hsa-mir-106b |
| KHK_miR_2306 | SEQ ID NO: 1299 | pre-hsa-mir-125a |

TABLE 1-40

| nucleic acid | | micro-RNA |
|---|---|---|
| name | micro-RNA | precursor |
| KHK_miR_2307 | SEQ ID NO: 1300 | pre-hsa-mir-128a |
| KHK_miR_2308 | SEQ ID NO: 1301 | pre-hsa-mir-130b |
| KHK_miR_2309 | SEQ ID NO: 1302 | pre-hsa-mir-132 |
| KHK_miR_2310 | SEQ ID NO: 1303 | pre-hsa-mir-141 |
| KHK_miR_2311 | SEQ ID NO: 1304 | pre-hsa-mir-148a |
| KHK_miR_2312 | SEQ ID NO: 1305 | pre-hsa-mir-15a |
| KHK_miR_2313 | SEQ ID NO: 1306 | pre-hsa-mir-16-1 |
| KHK_miR_2314 | SEQ ID NO: 1307 | pre-hsa-mir-16-2 |
| KHK_miR_2315 | SEQ ID NO: 1308 | pre-hsa-mir-196b |
| KHK_miR_2316 | SEQ ID NO: 1309 | pre-hsa-mir-19b-1 |
| KHK_miR_2317 | SEQ ID NO: 1310 | pre-hsa-mir-210 |
| KHK_miR_2318 | SEQ ID NO: 1311 | pre-hsa-mir-22 |
| KHK_miR_2319 | SEQ ID NO: 1312 | pre-hsa-mir-221 |
| KHK_miR_2320 | SEQ ID NO: 1313 | pre-hsa-mir-223 |
| KHK_miR_2321 | SEQ ID NO: 1314 | pre-hsa-mir-24-2 |
| KHK_miR_2322 | SEQ ID NO: 1315 | pre-hsa-mir-25 |
| KHK_miR_2323 | SEQ ID NO: 1316 | pre-hsa-mir-26a-2 |
| KHK_miR_2324 | SEQ ID NO: 1317 | pre-hsa-mir-26b |
| KHK_miR_2325 | SEQ ID NO: 1318 | pre-hsa-mir-29a |
| KHK_miR_2326 | SEQ ID NO: 1319 | pre-hsa-mir-29b-1 |
| KHK_miR_2327 | SEQ ID NO: 1320 | pre-hsa-mir-30c-1 |
| KHK_miR_2328 | SEQ ID NO: 1321 | pre-hsa-mir-30d |
| KHK_miR_2329 | SEQ ID NO: 1322 | pre-hsa-mir-33 |
| KHK_miR_2330 | SEQ ID NO: 1323 | pre-hsa-mir-339 |
| KHK_miR_2331 | SEQ ID NO: 1324 | pre-hsa-mir-340 |
| KHK_miR_2332 | SEQ ID NO: 1325 | pre-hsa-mir-342 |
| KHK_miR_2333 | SEQ ID NO: 1326 | pre-hsa-mir-34a |
| KHK_miR_2334 | SEQ ID NO: 1327 | pre-hsa-mir-361 |
| KHK_miR_2336 | SEQ ID NO: 1328 | pre-hsa-mir-500 |
| KHK_miR_2337 | SEQ ID NO: 1329 | pre-hsa-mir-93 |
| KHK_miR_2338 | SEQ ID NO: 1330 | pre-hsa-mir-140 |
| KHK_miR_2339 | SEQ ID NO: 1331 | pre-hsa-mir-151 |
| KHK_miR_2340 | SEQ ID NO: 1332 | pre-hsa-mir-181b-1 |
| KHK_miR_2341 | SEQ ID NO: 1333 | pre-hsa-mir-29b-2 |
| KHK_miR_2342 | SEQ ID NO: 1334 | pre-hsa-mir-29c |
| KHK_miR_2343 | SEQ ID NO: 1335 | pre-hsa-mir-423 |
| KHK_miR_2344 | SEQ ID NO: 1336 | pre-hsa-mir-7-1 |

As nucleic acids of the present invention, a nucleic acid consisting of a nucleotide sequence complementary to a nucleic acid mentioned above, and a double-stranded nucleic acid consisting of a nucleic acid mentioned above and a nucleic acid consisting of a nucleotide sequence complementary to the nucleotide sequence of the nucleic acid can also be mentioned.

In the present invention, a nucleic acid consisting of a nucleotide sequence having an identity of 90% or more to the nucleotide sequence of any one of SEQ ID NOs:1 to 1336 means a nucleic acid having an identity of at least 90% or more, preferably 91% or more, more preferably 92% or more, still more preferably 93% or more, particularly preferably 94% or more, and most preferably 95% or more, to a nucleic acid consisting of the nucleotide sequence of any one of SEQ ID NOs:1 to 1336, as calculated using an analytical software program such as BLAST [J. Mol. Biol., 215, 403 (1990)] or FASTA [Methods in Enzymology, 183, 63 (1990)]. In the present invention, a nucleic acid consisting of a nucleotide sequence having an identity of 80% or more to the nucleotide sequence of any one of SEQ ID NOs:1337 to 2851 means a nucleic acid having an identity of at least 80% or more, preferably 85% or more, more preferably 90% or more, still more preferably 92% or more, particularly preferably 95% or more, and most preferably 96% or more, to a nucleic acid consisting of the nucleotide sequence of any one of SEQ ID NOs:1337 to 2851, as calculated using an analytical software program such as BLAST [J. Mol. Biol., 215, 403 (1990)] or FASTA [Methods in Enzymology, 183, 63 (1990)].

In the present invention, a nucleic acid that hybridizes under stringent conditions includes, for example, a nucleic acid that can be identified by adding a probe RNA labeled γ-³²P-ATP to a Hybridization buffer consisting of 7.5 mL of 20×SSC, 0.6 mL of 1 M Na₂HPO₄ (pH 7.2), 21 mL of 10% SDS, 0.6 mL of 50× Denhardt′s solution, and 0.3 mL of 10 mg/mL sonicated salmon sperm DNA, wherein the probe is a nucleic acid having the nucleotide sequence of any of SEQ ID NOs:1 to 2851 or a partial fragment thereof, carrying out a reaction at 50° C. overnight, thereafter washing the membrane with 5×SSC/5% SDS liquid at 50° C. for 10 minutes, and further washing the same with 1×SSC/1% SDS liquid at 50° C. for 10 minutes, thereafter taking out the membrane, and applying it to an X-ray film.

In the present invention, the nucleic acid may be any molecule, as far as it is a molecule resulting from polymerization of a nucleotide or a molecule functionally equivalent to the nucleotide; for example, an RNA, which is a ribonucleotide polymer, a DNA, which is a deoxyribonucleotide polymer, a mixed polymer of RNA and DNA, and a nucleotide polymer, including a nucleotide analogue, can be mentioned; furthermore, the nucleic acid may be a nucleotide polymer, including a nucleic acid derivative, and may be a single-stranded nucleic acid or a double-stranded nucleic acid. A micro-RNA or a derivative thereof and a micro-RNA precursor or a derivative thereof are also included in nucleic acids of the present invention.

In the present invention, the nucleotide analogue may be any molecule, as far as it is a molecule prepared by modifying a ribonucleotide, a deoxyribonucleotide, an RNA or a DNA in order to improve the nuclease resistance thereof, to stabilize the same, to increase the affinity thereof for a complementary chain nucleic acid, to increase the cell permeability thereof, or to visualize the same, compared with the RNA or DNA; the analogue may be a naturally occurring molecule or a non-natural molecule; for example, a nucleotide analogue modified at the sugar moiety thereof, a nucleotide analogue modified by phosphodiester binding and the like can be mentioned.

The nucleotide analogue modified at the sugar moiety thereof may be any one, as far as an optionally chosen chemical structural substance has been added to, or substituted for, a portion or all of the chemical structure of the sugar of the nucleotide; for example, a nucleotide analogue substituted by 2′-O-methylribose, a nucleotide analogue substituted by 2′-O-propylribose, a nucleotide analogue substituted by 2′-methoxyethoxyribose, a nucleotide analogue substituted by 2′-O-methoxyethylribose, a nucleotide analogue substituted by 2′-O-[2-(guanidium)ethyl]ribose, a nucleotide analogue substituted by 2′-O-fluororibose, a bridged nucleic acid (BNA) having two cyclic structures as a result of introduction of a bridging structure into the sugar moiety, more specifically a locked nucleic acid (LNA) wherein the oxygen atom at the 2′ position and the carbon atom at the 4′ position have been bridged via methylene, and an ethylene bridged nucleic acid (ENA) [Nucleic Acid Research, 32, e175 (2004)] can be mentioned, and a peptide nucleic acid (PNA) [Acc. Chem. Res., 32, 624 (1999)], an oxypeptide nucleic acid (OPNA) [J. Am. Chem. Soc., 123, 4653 (2001)], and a peptide ribonucleic acid (PRNA) [J. Am. Chem. Soc., 122, 6900 (2000)] and the like can also be mentioned.

The nucleotide analogue modified by phosphodiester binding may be any one, as far as an optionally chosen chemical substance has been added to, or substituted for, a portion or all of the chemical structure of the phosphodiester bond of the nucleotide; for example, a nucleotide analogue substituted by a phosphorothioate bond, a nucleotide analogue substituted by an N3′-P5′ phosphoamidate bond, and the like can be mentioned [SAIBO KOGAKU, 16, 1463-1473 (1997)] [RNAi Method and Antisense Method, Kodansha (2005)].

In the present invention, the nucleic acid derivative may be any molecule, as far as it is a molecule prepared by adding another chemical substance to the nucleic acid in order to improve the nuclease resistance thereof, to stabilize the same, to increase the affinity thereof for a complementary chain nucleic acid, to increase the cell permeability thereof, or to visualize the same, compared with the nucleic acid; for example, a 5′-polyamine addition derivative, a cholesterol addition derivative, a steroid addition derivative, a bile acid addition derivative, a vitamin addition derivative, a Cy5 addition derivative, a Cy3 addition derivative, a 6-FAM addition derivative, a biotin addition derivative and the like can be mentioned.

As examples of the other nucleic acid derivatives, specifically as derivatives modified at the sugar moiety, an oligonucleotide derivative substituted by 2′-O-propylribose, an oligonucleotide derivative substituted by 2′-methoxyethoxyribose, an oligonucleotide derivative substituted by 2′-O-methylribose, an oligonucleotide derivative substituted by 2′-O-methoxyethylribose, an oligonucleotide derivative substituted by 2′-O-[2-(guanidium)ethyl]ribose, an oligonucleotide derivative substituted by 2′-O-fluororibose and the like can be mentioned; as derivatives modified at the phosphate group, an oligonucleotide derivative wherein a phosphodiester bond in an oligonucleotide has been converted to a phosphorothioate bond, an oligonucleotide derivatives wherein a phosphodiester bond in an oligonucleotide has been converted to an N3′-P5′phosphoamidate bond and the like can be mentioned [SAIBO KOGAKU, 16, 1463-1473 (1997)] [RNAi Method and Antisense Method, Kodansha (2005)].

In the present invention, the micro-RNA derivative may be any polymer comprising a molecule, other than a ribonucleotide, that is functionally equivalent to the micro-RNA; for example, a DNA, which is a deoxyribonucleotide polymer, a mixed polymer of RNA and DNA, and a nucleotide polymer, including a nucleotide analogue, can be mentioned; furthermore, the micro-RNA derivative may be a nucleotide polymer, including a nucleic acid derivative, and may be a single-stranded nucleic acid or a double-stranded nucleic acid.

The micro-RNA precursor derivative may be any one, as far as it is a polymer comprising a molecule, other than a ribonucleotide, that is functionally equivalent to the micro-RNA precursor; for example, a DNA, which is a deoxyribonucleotide polymer, a mixed polymer of RNA and DNA, and a nucleotide polymer, including a nucleotide analogue, can be mentioned; furthermore, the micro-RNA precursor derivative may be a nucleotide polymer, including a nucleic acid derivative, and may be a single-stranded nucleic acid or a double-stranded nucleic acid.

The method of producing a nucleic acid of the present invention is not particularly limited; the same can be produced by a method using a known chemical synthesis, or an enzymatic transcription method and the like. As methods using a known chemical synthesis, the phosphoroamidite method, the phosphorothioate method, the phosphotriester method, the CEM method [Nucleic Acid Research, 35, 3287 (2007)] and the like can be mentioned; for example, the same can be synthesized using the ABI3900 high throughput nucleic acid synthesizer (manufactured by Applied Biosystems). As an enzymatic transcription method, transcription with a plasmid or DNA having a desired nucleotide sequence as the template using a typical phage RNA polymerase, for example, T7, T3, or SP6RNA polymerase, can be mentioned.

The method of detecting the expression of a nucleic acid such as a micro-RNA or a micro-RNA precursor using a nucleic acid of the present invention may be any method that enables detection of a nucleic acid in a sample; for example, (1) Northern hybridization, (2) dot blot hybridization, (3) in situ hybridization, (4) quantitative PCR, (5) differential hybridization, (6) microarray, (7) ribonuclease protection assay and the like can be mentioned.

The method of detecting a mutation of a nucleic acid such as a micro-RNA or a micro-RNA precursor using a nucleic acid of the present invention may be any method that enables detection of a mutation of the nucleotide sequence of a nucleic acid in a sample; for example, a method wherein a heteroduplex formed by hybridization of a nucleic acid having a non-mutated nucleotide sequence and a nucleic acid having a mutated nucleotide sequence are detected, or a method wherein a sample-derived nucleotide sequence is directly sequenced to detect the presence or absence of a mutation and the like can be mentioned.

The method of separating a cell that expresses a nucleic acid such as a micro-RNA or a micro-RNA precursor using a nucleic acid of the present invention may be any method that enables separation of a cell that expresses a nucleic acid such as a micro-RNA or a micro-RNA precursor from a mixture of various cells; for example, a method wherein a probe prepared by fluorescently labeling a nucleic acid having a sequence complementary to the nucleotide sequence of a nucleic acid of the present invention is introduced into a cell to cause hybridization with the probe, and only the cells that have hybridized with the labeled probe are separated using a flow cytometer with sorting function, and the like can be mentioned.

A vector that expresses a nucleic acid of the present invention refers to a vector designed for a nucleic acid of the present invention to be biosynthesized by being transcribed in a cell or in vitro, and the vector may be any vector having a promoter capable of transcribing a nucleic acid of the present invention in a cell or in vitro. Specifically, pcDNA6.2-GW/miR (manufactured by Invitrogen), pSilencer 4.1-CMV (manufactured by Ambion), pSINsi-hH1 DNA (manufactured by Takara Bio Inc.), pSINsi-hU6 DNA (manufactured by Takara Bio Inc.), pENTR/U6 (manufactured by Invitrogen) and the like can be mentioned.

The method of suppressing the expression of a gene having a target nucleotide sequence of a nucleic acid, such as a micro-RNA, of the present invention (hereinafter referred to as a target gene) may be any method that suppresses the expression of a target gene by means of the activity to suppress the expression of an mRNA having a target nucleotide sequence using a nucleic acid, such as a micro-RNA, of the present invention. Here, to suppress the expression encompasses a case where the translation of an mRNA is suppressed, and a case where cleavage or decomposition of an mRNA results in a decreased amount of protein translated from the mRNA.

A target nucleotide sequence refers to the nucleotide sequence of a nucleic acid consisting of several nucleotides recognized by a nucleic acid, such as a micro-RNA, of the present invention. The translation of an mRNA having the nucleotide sequence is suppressed by a nucleic acid, such as a micro-RNA, of the present invention. Because an mRNA having a nucleotide sequence complementary to the sequence of the 2nd to 8th nucleotides on the 5′ terminal side of a micro-RNA undergoes suppression of the translation thereof by the micro-RNA [Current Biology, 15, R458-R460 (2005)], a nucleotide sequence complementary to the sequence of the 2nd to 8th nucleotides on the 5′ terminal side of a nucleic acid, such as a micro-RNA, of the present invention, can be mentioned as a target nucleotide sequence of the nucleic acid, such as the micro-RNA. For example, by providing a target sequence complementary to the 2nd to 8th nucleotides on the 5′ terminal side of a micro-RNA, and selecting an mRNA comprising a sequence completely identical to a set of 3′ UTR nucleotide sequences of human mRNAs by a method such as character sequence search, the target nucleotide sequence can be determined. A set of 3′ UTR nucleotide sequences of human mRNAs can be prepared using information on genome sequences and gene positions that can be acquired from “UCSC Human Genome Browser Gateway (http://genome.ucsc.edu/cgi-bin/hgGateway)”. As specific examples of genes having a target nucleotide sequence of a micro-RNA of SEQ ID NOs:1 to 1336, the genes shown in Tables 2-1 to 2-35, represented by names (Official Symbols and Gene IDs) used in the EntreGene database (http://www.ncbi.nlm.nih.gov/Entrez/) of the US National Center for Biotechnology Information (NCBI), can be mentioned. The gene names used are names in the EntreGene database as of March 2006.

TABLE 2-1

| SEQ | |
|---|---|
| ID NO | Target gene |
| 1 | PRKCZ(5590); TNFRSF14(8764); TNFRSF9(3604); RCC2(55920); NBL1(4681); LDLRAD2(401944); CDC42(998); E2F2 |
| | (1870); RCC1(1104); SDC3(9672); KHDRBS1(10657); HPCA(3208); RIMS3(9783); TOE1(114034); SERBP1(26135); |
| | SYPL2(284612); SLC6A17(388662); MCL1(4170); TNRC4(11189); C1orf60(65123); IL6R(3570); ZBTB7B(51043); LMNA |
| | (4000); MEF2D(4209); PEA15(8682); DEDD(9191); QSCN6(5768); ADORA1(134); IL24(11009); TMEM63A(9725); RHOB |
| | (388); KCNK3(3777); ZFP36L2(678); HTRA2(27429); ADRA2B(151); BRRN1(23397); BCL2L11(10018); BBS5(129880); |
| | CASP10(843); ALS2CR19(117583); INHA(3623); HDAC4(9759); KIF1A(547); SEPT2(4735); NEU4(129807); ZAK |
| | (51776); OGG1(4968); HDAC11(79885); ZNF651(92999); ZNF445(353274); DAG1(1605); HEMK1(51409); RBM15B |
| | (29890); RNF7(9616); ALG3(10195); TP73L(8626); FLJ37478(339983); LRPAP1(4043); APBB2(323); PRLR(5618); ARSB |
| | (411); SEMA6A(57556); RAD50(10111); PFDN1(5201); WDR55(54853); N4BP3(23138); ATXN1(6310); E2F3(1871); |
| | IER3(8870); MCCD1(401250); C6orf47(57827); ZBTB12(221527); CREBL1(1388); PBX2(5089); BAK1(578); IHPK3(117283); |
| | CDKN1A(1026); ZFAND3(60685); KCNK5(8645); NFYA(4800); FOXO3A(2309); SNX8(29888); TRIAD3(54476); |
| | CARD4(10392); INHBA(3624); DKFZp761I2123(83637); ADCY1(107); YWHAG(7532); FLJ39237(375607); LRRC4(64101); |
| | EPHA1(2041); CDK5(1020); TNFRSF10B(8795); TNFRSF10C(8794); PTK2B(2185); KIF13B(23303); UNC5D(137970); |
| | EXT1(2131); DGAT1(8694); MGC70857(414919); GAS1(2619); SEMA4D(10507); TRAF1(7185); CDK9(1025); |
| | ST6GALNAC6(30815); ABL1(25); BRD3(8019); TRAF2(7186); C9orf140(89958); FLJ20245(54863); FRMD4A(55691); |
| | SPOCK2(9806); LOC439985(439985); PTEN(5728); SLIT1(6585); NOLC1(9221); C10orf46(143384); FAM53B(9679); |
| | FLJ46300(399827); MUCDHL(53841); TOLLIP(54472); BRSK2(9024); IGF2(3481); OSBPL5(114879); C11orf49(79096); |
| | VEGFB(7423); CDCA5(113130); DPF2(5977); SSSCA1(10534); RELA(5970); PPP1CA(5499); RPS6KB2(6199); |
| | CCND1(595); FADD(8772); INPPL1(3636); GAB2(9846); SCN4B(6330); ABCG4(64137); EI24(9538); ST3GAL4(6484); |
| | CACNA1C(775); TSPAN9(10867); CCND2(894); ING4(51147); PTMS(5763); FGD4(121512); FLJ20489(55652); HDAC7A |
| | (51564); RND1(27289); MCRS1(10445); FAIM2(23017); RARG(5916); GDF11(10220); CENTG1(116986); PPTC7 |
| | (160760); PPP1CC(5501); SCARB1(949); RAN(5901); DIP13B(55198); SLC7A1(6541); FLJ40296(122183); POU4F1(5457); |
| | LAMP1(3916); BCL2L2(599); TM9SF1(10548); FLJ39779(400223); ERH(2079); VASH1(22846); CHES1(1112); TRAF3 |
| | (7187); FLJ42486(388021); AKT1(207); NDN(4692); KLF13(51621); BMF(90427); BAHD1(22893); CSK(1445); IGF1R |
| | (3480); PIGQ(9091); KIAA1924(197335); CCDC78(124093); C1QTNF8(390664); NME3(4832); TBL3(10607); TSC2 |
| | (7249); TRAF7(84231); CCNF(899); TNFRSF12A(51330); MMPL1(4328); N-PAC(84656); GPRC5B(51704); PLK1(5347); |
| | MAPK3(5595); FBXL19(54620); POLR2C(5432); TRADD(8717); LOC283849(283849); E2F4(1874); SLC12A4(6560); |
| | SLC7A6(9057); SMPD3(55512); TERF2(7014); KIAA0513(9764); PCOLN3(5119); PRDM7(11105); PARD6A(50855); |
| | ALOX15B(247); PAFAH1B1(5048); DHX33(56919); VAMP2(6844); GAS7(8522); CORO6(84940); GOSR1(9527); |
| | RASL10B(91608); DUSP3(1845); UBTF(7343); SLC4A1(6521); MAPT(4137); CDK5RAP3(80279); CROP(51747); MSI2 |
| | (124540); ERN1(2081); PRKCA(5578); SPHK1(8877); TMC6(11322); BIRC5(332); SOCS3(9021); FLJ21865(64772); |
| | CARD14(79092); CIDEA(1149); CABLES1(91768); MAPRE2(10982); BCL2(596); TXNL4A(10907); APC2(10297); MKNK2 |
| | (2872); C19orf36(113177); FZR1(51343); MATK(4145); DAPK3(1613); ZBTB7A(51341); TNFAIP8L1(126282); MAPK7 |
| | (5609); CDKN2D(1032); TNPO2(30000); GADD45GIP1(90480); BTBD14B(112939); DPF1(8193); MAP3K10(4294); |
| | AKT2(208); PRX(57716); AXL(558); ARHGEF1(9138); ERF(2077); IGSF4C(199731); APOE(348); SPHK2(56848); |
| | RPS11(6205); SPIB(6689); LOC284361(284361); SAPS1(22870); HMG20B(10362); GRLF1(2909); CSNK2A1(1457); |
| | TM9SF4(9777); E2F1(1869); GGTL3(2686); LPIN3(64900); MYBL2(4605); DIDO1(11083); BIRC7(79444); ARFGAP1 |
| | (55738); PLAC4(191585); FLJ41733(400870); C21orf123(378832); BCL2L13(23786); TSSK2(23617); DGCR14(8220); |
| | LOC128977(128977); SEPT5(5413); HIC2(23119); MAPK1(5594); SMARCB1(6598); ADORA2A(135); MN1(4330); GAS2L1 |
| | (10634); CABP7(164633); KIAA1904(114794); CBX6(23466); CBX7(23492); MGAT3(4248); PPARA(5465); FLJ41993 |
| | (400935); SAPS2(9701); SBF1(6305); MAPK8IP2(23542); MGC3731(79159); PCTK1(5127); SMC1L1(8243); :SNX12 |
| | (29934); BTK(695); AGTR2(186); ZDHHC9(51114); EMD(2010); AFF2(2334); |
| 2 | E2F2(1870); CCDC21(64793); SDC3(9672); RBBP4(5928); KIAA0319L(79932); C1orf84(149469); AGBL4(84871); CELSR2 |
| | (1952); RAP1A(5906); SV2A(9900); C1orf60(65123); SHC1(6464); UBQLN4(56893); MEF2D(4209); BCAN(63827); |
| | ISF4B(57863); NAV1(89796); PPP1R12B(4660); RIPK5(25778); C1orf95(375057); YPEL5(51646); TEX261(113419); |
| | KCNIP3(30818); IMP4(92856); PNKD(25953); ATG9A(79065); COPS7B(64708); ATP2B2(491); HEMK1(51409); RAB6B |
| | (51560); SLCO2A1(6578); SLC26A1(10861); EVC(2121); FLJ46481(389197); SETD7(80854); KIAA1909(153478); |
| | PCDH1(5097); PDGFRB(5159); NDST1(3340); CPLX2(10814); N4BP3(23138); RNF5(6048); PBX2(5089); BAK1(578); |
| | PACSIN1(29993); MOCS1(4337); FOXP4(116113); MGG45491(221416); TRAM2(9697); PSCD3(9265); AQP1(358); |
| | POM121(9883); CASP2(835); VIPR2(7434); C9orf25(203259); TPM2(7169); NTRK2(4915); C9orf47(286223); ST6GALNAC6 |
| | (30815); SPOCK2(9806); SH3PXD2A(9644); VAX1(11023); MTG1(92170); SPRN(503542); C11orf11(747); PACS1 |
| | (55690); LOC399947(399947); BCL9L(283149); CACNA1C(775); FLJ20489(55652); FMNL3(91010); CBX5(23468); |
| | MYL6(4637); GLS2(27165); CTDSP2(10106); DDX54(79039); B4GALNT1(2583); SLC8A3(6547); PCNX(22990); |
| | KIAA0125(9834); BMF(90427); TLN2(83660); PML(5371); NEIL1(79661); ABHD2(11057); EMP2(2013); UBPH(56061); |
| | SBK1(388228); ATP2A(487); N4BP1(9683); CCL22(6367); POLR2C(5432); AFG3L1(172); SMG6(23293); GAS7(8522); |
| | C17orf63(55731); ACACA(31); MLLT6(4302); LASP1(3927); WIRE(147179); PTRF(284119); MAPT(4137); PNPO(55163); |
| | NGFR(4804); NXPH3(11248); LIMD2(80774); CDC42EP4(23580); C17orf28(283987); SEPT9(10801); PCYT2(5833); |
| | MAFG(4097); RNF165(494410); FZR1(51343); TUBB4(10382); PDE4A(5141); NFIX(4784); RAB8A(4218); ZNF585A |
| | (199704); APOE(348); DMWD(1762); SHANK1(50944); MGC2752(65996); LOC389286(389286); C20orf27(54976); |
| | SOX12(6666); ProSAPiP1(9762); P$$MS4(140730); DIDO1(11083); BTBD4(140685); TRPM3(7226); POFUT2(23275); |
| | MICAL-L1(85377); MGAT3(4248); TEF(7008); NFAM1(150372); TTLL12(23170); SSX4B(548313); SYN1(6853); SSX |

TABLE 2-2

| | |
|---|---|
| | 6(280657); RP11-114H20.1(401589); SSX2(6757); SMC1L1(8243); |
| 3 | TNFRSF14(8764); NBL1(4681); CDC42(998); LIN28(79727); KHDRBS1(10657); JMJD2A(9682); GFI1(2672); CDC14A |
| | (8556); AMIGO1(57463); MCL1(4170); RORC(6097); MEF2D(4209); PEA15(8682); UHMK1(127933); ADORA1(134); |
| | NUCKS1(64710); TP53BP2(7159); C1orf69(200205); KIF21B(23046); RHOB(388); KCNK3(3777); PRKCE(5581); MXD1 |
| | (4084); KCNIP3(30818); BRRN1(23397); BBS5(129880); CFLAR(8837); CASP10(843); CTDSP1(58190); INHA(3623); |
| | PAX3(5077); HDAC4(9759); KIF1A(547); SRGAP3(9901); VHL(7428); RBMS3(27303); AXUD1(64651); ZNF651(92999); |
| | RBM15B(29890); MAGI1(9223); FXR1(8087); TP73L(8626); PPP1R2(5504); G3BP2(9908); PRLR(5618); FLJ39155 |
| | (133584); ITGA2(3673); RAD17(5884); SEMA6A(57556); RAD50(10111); PHF15(23338); HNRPA0(10949); CDC23 |
| | (8697); CAMK2A(815); CD74(972); NDST1(3340); DDR1(780); ZBTB12(221527); SKIV2L(6499); SYNGAP1(8831); BAK1 |
| | (578); MAPK13(5603); ZFAND3(60685); TRAM2(9697); FOXO3A(2309); L3MBTL3(84456); STX7(8417); TNFAIP3 |
| | (7128); PDE10A(10846); PDCD2(5134); TRIAD3(54476); SCRN1(9805); IGFBP3(3486); EGFR(1956); YWHAG(7532); LEP |
| | (3952); FASTK(10922); TNFRSF10B(8795); TNFRSF10A(8797); TRIM35(23087); CLU(1191); KIF13B(23303); DUSP4 |
| | (1846); UBE2V2(7336); MTSS1(9788); CDC37L1(55664); CDK9(1025); C9orf140(89958); GPR107(57720); ADARB2 |
| | (105); SPOCK2(9806); NOLC1(9221); TCF7L2(6934); C10orf46(143384); TIAL1(7073); LRRC56(115399); IGF2(3481); |
| | IPO7(10527); API5(8539); PRPF19(27339); GANAB(23193); CDCA5(113130); DPF2(5977); SSSCA1(10534); NPAS4 |
| | (266743); PPP1CA(5499); FGF4(2249); SESN3(143686); YAP1(10413); PPP2R1B(5519); FLJ25530(220296); EI24 |
| | (9538); ARF3(377); C12orf22(81566); LOC401720(401720); GDF11(10220); SUDS3(64426); HNRPA1(3178); LATS2 |
| | (26524); FLJ40296(122183); POU4F1(5457); BCL2L2(599); JPH4(84502); ERH(2079); VASH1(22846); CHES1(1112); |
| | ANKRD9(122416); AKT1(207); KLF13(51621); TPM1(7168); CSK(1445); IGF1R(3480); CCDC78(124093); C1QTNF8 |
| | (390664); TSC2(7249); TRAF7(84231); TNFRSF12A(51330); ERN2(10595); MAPK3(5595); FBXL19(54620); CIAPIN1 |
| | (57019); CMTM4(146223); TRADD(8717); LOC283849(283849); SLC12A4(6560); SMPD3(55512); KIAA0513(9764); CDT1 |
| | (81620); RPL13(6137); ALOX15B(247); MNT(4335); PAFAH1B1(5048); DLG4(1742); TNFSF12(8742); GOSR1(9527); |
| | NF1(4763); RAB11FIP4(84440); RASL10B(91608); NR1D1(9572); HDAC5(10014); CRHR1(1394); MAPT(4137); |
| | NGFR(4804); SPHK1(8877); TMC6(11322); SOCS3(9021); PCYT2(5833); CIDEA(1149); CABLES1(91768); PHLPP(23239); |
| | BCL2(596); TXNL4A(10907); FZR1(51343); ZBTB7A(51341); SEMA6B(10501); TNFSF9(8744); CDC37(11140); |
| | CDKN2D(1032); ILF3(3609); DNM2(1785); RAB3D(9545); LPPR2(64748); ELAVL3(1995); TNPO2(30000); BTBD14B |
| | (112939); F2RL3(9002); ELL(8178); DPF1(8193); MAP3K10(4294); ERF(2077); IGSF4C(199731); BCL3(602); APOE |
| | (348); FOSB(2354); BBC3(27113); SLC8A2(6543); BAX(581); CD37(951); BCL2L12(83596); PPP2R1A(5518); MYADM |
| | (91663); CACNG7(59284); U2AF2(11338); ZNF264(9422); ZNF17(7565); GRLF1(2909); SCRT2(85508); E2F1(1869); |
| | UBE2V1(7335); CABLES2(81928); DIDO1(11083); ADARB1(104); BCL2L13(23786); DGCR14(8220); SEPT5(5413); |
| | MAPK1(5594); SMARCB1(6598); MN1(4330); GAS2L1(10634); LIF(3976); MCM5(4174); KIAA1904(114794); CARD10 |
| | (29775); C22orf5(25829); CBX6(23466); CBX7(23492); TCF20(6942); PHF21B(112885); PPARA(5465); SAPS2(9701); |
| | SBF1(6305); MAPK8IP2(23542); RS1(6247); PCTK1(5127); ELK1(2002); ZNF81(347344); SMO1L1(8243); RP1-112K5.2 |
| | (90121); MLLT7(4303); BRWD3(254065); SEPT6(23157); MTCP1(4515); AFF2(2334); |
| 4 | BOK(666) |
| 5 | GAS7(8522) |
| 6 | PI15(51050) |
| 7 | TAGLN(6876) |
| 8 | SAMD11(148398); CDC2L2(985); TP73(7161); HES2(54626); THAP3(90326); TNFRSF9(3604); LDLRAP1(26119); NUDC |
| | (10726); RCC1(1104); TRIM62(55223); KIAA0319L(79932); MPL(4352); CDC20(991); SH3GLB1(51100); PROK1 |
| | (84432); KCNJ9(3765); DEDD(9191); GPR161(23432); TNNI1(7135); TMEM63A(9725); C1Oorf95(375057); C1orf69(200205); |
| | MTR(4548); RHOB(388); CGREF1(10669); BRRN1(23397); BCL2L11(10018); WDR33(55339); PLEKHB2(55041); |
| | MCM6(4175); KIF1A(547); RAF1(5894); AXUD1(64651); RASSF1(11186); NEK4(6787); TMEM110(375346); CLSTN2 |
| | (64084); LOC285382(285382); LETM1(3954); SH3BP2(6452); HD(3064); BTC(685); UNC5C(8633); NEK1(4750); TPPP |
| | (11076); ERBB2IP(55914); MRPS27(23107); CDC23(8697); PDGFRB(5159); HMP19(51617); DHX16(8449); AIF1(199); |
| | MAPK13(5603); CDKN1A(1026); LATS1(9113); ESR1(2099); RPS6KA2(6196); LOC90639(90639); MAD1L1(8379); |
| | FOXK1(221937); TRIAD3(54476); HDAC9(9734); INHBA(3624); TMED4(222068); ADCY1(107); IGFBP3(3486); YWHAG |
| | (7532); KIAA0773(9715); LOC155060(155060); RBM33(155435); MAFK(7975); CLN8(2055); BIN3(55909); TNFRSF10C |
| | (8794); CLU(1191); BAG1(573); SHB(6461); C9orf47(286223); TRIM14(9830); TRAF1(7185); ASB6(140459); FNBP1 |
| | (23048); ABL1(25); TSC1(7248); QSCN6L1(169714); UNC5B(219699); C10orf104(119504); FAS(355); LZTS2(84445); |
| | SUFU(51684); SPRN(503542); IGF2(3481); KCNQ1(3784); WT1(7490); SLC15A3(51296); LOC144097(144097); |
| | CDC42EP2(10435); BRMS1(25855); CCND1(595); FADD(8772); LRRC51(220074); GAB2(9846); SESN3(143686); CASP5 |
| | (838); EXPH5(23086); TAGLN(6876); ETV6(2120); FLJ20489(55652); HDAC7A(51564); MCRS1(10445); FMNL3 |
| | (91010); FAIM2(23017); CBX5(23468); RBMS2(5939); BTG1(694); SOCS2(8835); GAS2L3(283431); TCF1(6927); C12orf43 |
| | (64897); DIABLO(56616); SCARB1(949); LATS2(26524); WDFY2(115825); LTB4R2(56413); SPTB(6710); RAB15 |
| | (376267); LTBP2(4053); VASH1(22846); SLC24A4(123041); TRAF3(7187); PACS2(23241); KIAA0125(9834); FLJ43339 |
| | (388115); CA12(771); DAPK2(23604); PDCD7(10081); CLN6(54982); ARNT2(9915); C15orf38(348110); IDH2(3418); |
| | IGF1R(3480); LOC440313(440313); LRRK1(79705); RAB40C(57799); KIAA1924(197335); CCDC78(124093); CLCN7 |
| | (1186); TRAF7(84231); CCNF(899); PKMYT1(9088); BTBD12(84464); DNAJA3(9093); C16orf5(29965); GSPT1(2935); |
| | XYLT1(64131); RPS15A(6210); PLK1(5347); SBK1(388228); STX1B2(112755); FTS(64400); GNAO1(2775); LIN1 |

TABLE 2-3

| | |
|---|---|
| | 0(80262); LOC283849(283849); RANBP10(57610); SLC12A4(6560); SMPD3(55512); KIAA0513(9764); FLJ45121(400556); |
| | CDT1(81620); CDK10(8558); ANAPC11(51529); ALOX15B(247); TUSC5(286753); HIC1(3090); TNFSF12(8742); |
| | GAS7(8522); MFAP4(4239); WSB1(26118); POLDIP2(26073); HCP1(113235); EZH1(2145); MAPT(4137); NFE2L1 |
| | (4779); CROP(51747); KIAA0195(9772); EXOC7(23265); TBC1D16(125058); BAIAP2(10458); DUS1L(64118); MAPK4 |
| | (5596); DCC(1630); MBP(4155); HMHA1(23526); ZBTB7A(51341); MAP2K7(5609); ELAVL3(1995); PRDX2(7001); F2RL3 |
| | (9002); KIAA0892(23383); ZNF43(7594); MGC20255(90324); APOE(348); LOC400707(400707); CARD8(22900); |
| | RPS11(6205); HKR2(342945); SNPH(9751); KCNK15(60598); CSE1L(1434); UBE2V1(7335); LAMA5(3911); DIDO1(11083); |
| | CHRNA4(1137); AGPAT3(56894); TRPM2(7226); UBE2G2(7327); COL18A1(80781); C22orf25(128989); HTF9C |
| | (27037); MAPK1(5594); PPM1F(9647); SMARCB1(6598); GAS2L1(10634); NF2(4771); SSTR3(6753); TOB2(10766); |
| | SEPT3(55964); PPARA(5465); GTSE1(51512); ADM2(79924); APOL6(80830); ZC3H12B(340554); RNF12(51132); MECP2 |
| | (4204); |
| 9 | GM632(57473) |
| 10 | CEBPA(1050) |
| 11 | RBPSUH(3516) |
| 12 | BOK(666) |
| 13 | PACS2(23241) |
| 14 | KIF1B(23095); SPATA6(54558); LEPR(3953); GBP6(163351); LRRC8C(84230); ABCD3(5825); AGL(178); CDC14A(8556); |
| | UHMK1(127933); RGS4(5999); YOD1(55432); NCOA1(8648); CRIM1(51232); FLJ13910(64795); HNMT(3176); LOC339745 |
| | (339745); ITGA4(3676); PGAP1(80055); CREB1(1385); TRPM8(79054); MGAT4A(11320); IGSF4D(253559); |
| | VGLL3(389136); ZPLD1(131368); IFT57(55081); NCK1(4690); DBR1(51163); DHX36(170506); PPAT(5471); TMPRSS11B |
| | (132724); RASSF6(166824); CXCL5(6374); PAQR3(152559); FGF5(2250); AFF1(4299); DAPP1(27071); DNAJB14 |
| | (79982); BBS7(55212); SLC7A11(23657); FLJ38482(201931); ASB5(140458); IL7R(3575); LIFR(3977); FLJ21657 |
| | (64417); HCN1(348980); ITGA2(3673); ELOVL7(79993); R7BP(401190); TNPO1(3842); POLK(51426); HOMER1(9456); |
| | LOC153364(153364); ACSL6(23305); FLJ37562(134553); DCDC2(51473); ICK(22858); ASCC3(10973); C6or68(116150); |
| | AHI1(54806); TXLNB(167838); PDE10A (10846); MGC42090(256130); CREB5(9586); LOC441257(441257); STEAP4 |
| | (79689); STEAP2(261729); BCAP29(55973); WNT16(51384); NDUFA5(4698); CALD1(800); PLAG1(5324); YTHDF3 |
| | (253943); PMP2(5375); ANGPT1(284); TRPS1(7227); KLHL9(55958); TRPM6(140803); FRMD3(251019); NTRK2 |
| | (4915); C9orf5(23731); PAPPA(5069); RP11-142I17.1(26095); PLEKHK1(219790); FAS(355); IFIT5(24138); ADD3(120); |
| | PGM2L1(283209); HBXAP(51773); PANX1(24145); SESN3(143686); KIAA0999(23387); CBL(867); KRAS(3845); |
| | STK38L(23012); MON2(23041); LIN7A(8825); SLC41A2(84102); TMEM132B(114795); MGC40069(348035); KIAA1333 |
| | (55632); MIPOL1(145282); C14orf138(79609); SOS2(6655); PIGH(5283); PCNX(22990); GTF2A1(2957); SEL1L(6400); |
| | UBE3A(7337); PLDN(26258); MYO5A(4644); USP31(57478); NF1(4763); MSI2(124540); C18orf1(753); DSG2(1829); |
| | SMAD2(4087); FVT1(2531); DOK6(220164); NETO1(81832); ZNF568(374900); BMP2(650); RAB22A(57403); BTBD4 |
| | (140685); CXADR(1525); PKNOX1(5316); KAL1(3730); DMD(1756); SCML1(6322); |
| 15 | RAB40C(57799) |
| 16 | NFAT5(10725) |
| 17 | LAMP1(3916) |
| 18 | PRX(57716) |
| 19 | GIPC3(126326) |
| 20 | TNFRSF1B(7133); RCC2(55920); TTMB(399474); FABP3(2170); GIPC2(54810); CDC14A(8556); CDC42SE1(56882); |
| | DEDD(9191); FASLG(356); ZBTB41(360023); TNNI1(7135); LPGAT1(9926); C1orf140(400804); NID1(4811); AKT3(10000); |
| | C1orf173(127254); SOX11(6664); CAD(790); FOSL2(2355); REEP1(65055); IL1RL1(9173); IL1A(3552); PTD004 |
| | (29789); CASP10(843); CREB1(1385); SEPT2(4735); ZAK(51776); RBMS3(27303); EIF4E3(317649); KALRN(8997); |
| | SLO9A9(285195); SLC2A2(6514); TNFSF10(8743); DKFZP686A01247(22998); GABRA4(2557); FLJ38991(285521); |
| | SNCA(6622); CASP6(839); ADCY2(108); PRLR(5618); LOC153561(153561); ZBED3(84327); APC(324); DNAJC18(202052); |
| | PCDHGB4(8641); UNC5A(90249); PRPF4B(8899); FLJ45422(441140); FKBP5(2289); RPL7L1(285855); SYNCRIP |
| | (10492); PLAGL1(5325); RABGEF1(27342); PODXL(5420); ABCF2(10061); REXO1L1(254958); DLGAP2(9228); |
| | EFHA2(286097); UNC5D(137970); CHD7(55636); MTFR1(9650); UBE2W(55284); ATP6V0D2(245972); CCNE2(9134); |
| | PTPLAD2(401494); SMU1(55234); TLR4(7099); MAP3K8(1326); C10orf54(64115); KIF11(3832); TIAL1(7073); GAS2 |
| | (2620); SSSCA1(10534); PGM2L1(283209); HBXAP(51773); NDUFC2(4718); DLG2(1740); MTMR2(8898); SNF1LK2 |
| | (23235); CBL(867); LOC283174(283174); KRAS(3845); CBX5(23468); RBMS2(5939); TMCC3(57458); KIAA0152(9761); |
| | TNFRSF19(55504); TFDP1(7027); RHOJ(57381); FLJ39531(400360); LOC405753(405753); PDCD7(10081); RAB11A |
| | (8766); AP3S2(10239); IQGAP1(8826); PHLPPL(23035); CROP(51747); WBP2(23558); DLGAP1(9229); C18orf1 |
| | (753); AQP4(361); SMAD2(4087); DOK6(220164); TNFSF14(8740); DNAJB1(3337); APOE(348); ZNF347(84671); RNF24 |
| | (11237); TP53INP2(58476); RBL1(5933); ABCC13(150000); NF2(4771); SYN3(8224); RAXLX(91464); ATRX(546); |

TABLE 2-4

| | CD40LG(959); AFF2(2334); |
|---|---|
| 21 | DFFA(1676); TNFRSF1B(7133); NBL1(4681); NUDC(10726); SESN2(83667); RCC1(1104); TTMB(399474); KIAA0319L |
| | (79932); NFYC(4802); TESK2(10420); CDC7(8317); RAP1A(5906); NRAS(4893); SNX27(81609); CD84(8832); UHMK1 |
| | (127933); QSCN6(5768); PPP1R12B(4660); CCNL2(81669); E2F6(1876); RBJ(51277); KHK(3795); OXER1(165140); |
| | TGFA(7039); KCNIP3(30818); BCL2L11(10018); ANAPC1(64682); RIF1(55183); CASP10(843); OGG1(4968); FANCD2 |
| | (2177); STAG1(10274); CHRD(8646); TP73L(8626); PCGF3(10336); CD38(952); EREG(2069); BTC(685); SEPT11(55752); |
| | KIAA1909(153478); UNG2(10309); F2R(2149); FGF1(2246); RNF130(55819); RIPK1(8737); LY86(9450); E2F3(1871); |
| | MAPK13(5603); TRAM2(9697); DST(667); SASH1(23328); FLJ34503(285759); MGC11257(84310); TRIAD3(54476); |
| | INHBA(3624); PURB(5814); CALN1(83698); CDK6(1021); CASP2(835); PSD3(23362); POLR3D(661); BIN3(55909); |
| | TNFRSF10B(8795); BNIP3L(665); CLU(1191); UNC5D(137970); UBE2V2(7336); STK3(6788); KIAA1875(340390); GAS1 |
| | (2619); SEMA4D(10507); FGD3(89846); TRIM14(9830); SPTAN1(6709); ADAMTS13(11093); CARD9(64170); ZMYND11 |
| | (10771); ZWINT(11130); SIRT1(23411); UNC5B(219699); LZTS2(84445); SUFU(51684); C10orf46(143384); HRAS |
| | (3265); PARVA(55742); HIPK3(10114); DAK(26007); DPF2(5977); DLG2(1740); SCN4B(6330); MLL(4297); H2AFX |
| | (3014); SRPR(6734); RAD52(5893); CCND2(894); TNFRSF7(939); CDKN1B(1027); IAPP(3375); HDAC7A(51564); FAIM2 |
| | (23017); C12orf22(81566); PDE1B(5153); DDIT3(1649); CENTG1(116986); PCTK2(5128); APAF1(317); HRK(8739); |
| | EP400NL(347918); TNFRSF19(55504); RB1(5925); KLF12(11278); CUL4A(8451); ACIN1(22985); BCL2L2(599); JPH4 |
| | (84502); MNAT1(4331); PSEN1(5663); TGFB3(7043); VASH1(22846); PPP1R13B(23368); MAPK6(5597); FLJ38723 |
| | (255180); KIF23(9493); IGF1R(3480); ALPK3(57538); RAB40C(57799); TRAF7(84231); PKMYT1(9088); DNAJA3 |
| | (9093); GSPT1(2935); PLK1(5347); ERN2(10595); PRRT2(112476); RNF40(9810); FTS(64400); CIAPIN1(57019); CTCF |
| | (10664); SMPD3(55512); CDK10(8558); ANAPC11(51529); MNT(4335); TNFSF12(8742); GAS7(8522); HCP1(113235); |
| | CDK5RAP3(80279); C17orf73(55018); MPO(4353); ABC1(63897); BIRC5(332); EMILIN2(84034); GNAL(2774); CABLES1 |
| | (91768); PHLPP(23239); BCL2(596); FZR1(51343); DPF1(8193); SIRT2(22933); SAMD4B(55095); LYPD5(284348); |
| | APOE(348); OPA3(80207); BBC3(27113); BAX(581); FLJ38288(284309); GRLF1(2909); PLCB1(23236); CSE1L |
| | (1434); ZGPAT(84619); MAPK1(5594); NF2(4771); CARD10(29775); NPTXR(23467); TNRC6B(23112); TOB2(10766); |
| | FIGF(2277); SMC1L1(8243); SEPT6(23157); MECP2(4204); |
| 22 | CDC2L2(985); TNFRSF14(8764); CHD5(26038); CDC42(998); E2F2(1870); WASF2(10163); RCC1(1104); KHDRBS1 |
| | (10657); LCK(3932); KIAA0319L(79932); RIMS3(9783); CDC20(991); CDKN2C(1031); NOTCH2(4853); CKS1B(1163); |
| | C1orf21(81563); PPP1R12B(4660); SYT2(127833); ADORA1(134); PIK3C2B(5287); IL10(3586); IL24(11009); PLXNA2 |
| | (5362); VANGL1(81839); KIF21B(23046); RHOB(388); MAPRE3(22924); TGFA(7039); HK2(3099); BRRN1(23397); INHBB |
| | (3625); ERCC3(2071); PROC(5624); TAIP-2(80034); TLK1(9874); BARD1(580); TNS1(7145); PAX3(5077); HDAC4 |
| | (9759); BOK(666); ZAK(51776); ZNF651(92999); RASSF1(11186); HEMK1(51409); RBM15B(29890); RNF7(9616); TP73L |
| | (8626); FAM43A(131583); LETM1(3954); HD(3064); EREG(2069); CCNG2(901); TPPP(11076); DAP(1611); PRKAA1 |
| | (5562); RPL37(6167); ERBB2IP(55914); APC(324); MCC(4163); HDAC3(8841); CPLX2(10814); NSD1(64324); IER3 |
| | (8870); C2(717); BAK1(578); IHPK3(117283); PACSIN1(29993); MAPK13(5603); CDKN1A(1026); TREML2(79865); PTK7 |
| | (5754); SRF(6722); VEGF(7422); SUPT3H(8464); IL17(3605); FOXO3A(2309); HECA(51696); ELMO1(9844); FOXK1 |
| | (221937); TRIAD3(54476); HDAC9(9734); ADCY1(107); IGFBP3(3486); EGFR(1956); FLJ37538(222950); CASP2(835); |
| | KIAA0773(9715); VIPR2(7434); LZTS1(11178); POLR3D(661); BIN3(55909); TNFRSF10A(8797); TRIM35(23087); |
| | PTK2B(2185); DUSP4(1846); UNC5D(137970); TERF1(7013); TP53INP1(94241); LYNX1(66004); MGC21881(389741); |
| | UHRF2(115426); CDKN2B(1030); TAF1L(138474); BAG1(573); FANCG(2189); SEMA4D(10507); TNFSF15(9966); TNFSF8 |
| | (944); TRAF1(7185); DAB2IP(153090); FAM102A(399665); ADARB2(105); ZWINT(11130); SIRT1(23411); SGPL1 |
| | (8879); C10orf54(64115); CHST3(9469); KIF11(3832); LZTS2(84445); FGF8(2253); C10orf76(79591); SUFU(51684); |
| | PDCD11(22984); NEURL(9148); BAG3(9531); FAM53B(9679); APBB1(322); MDK(4192); CDCA5(113130); DPF2(5977); |
| | CLCF1(23529); PPP1CA(5499); FADD(8772); RAB30(27314); SCN2B(6327); H2AFX(3014); EI24(9538); CDCA3 |
| | (83461); BCAT1(586); KRAS(3845); HNDAC7A(51564); MCRS1(10445); TEGT(7009); FAIM2(23017); CBX5(23468); PDE1B |
| | (5153); ITGA7(3679); MYL6(4637); CRADD(8738); FOXO1A(2308); RFP2(10206); POU4F1(5457); LOC283487(283487); |
| | BCL2L2(599); RGS6(9628); PSEN1(5663); LTBP2(4053); NEK9(91754); VASH1(22846); TRAF3(7187); BAG5 |
| | (9529); AKT1(207); JAG2(3714); BMF(90427); FLJ43339(388115); FLJ38723(255180); SNX1(6642); PDCD7(10081); |
| | CSK(1445); ABHD2(11057); IGF1R(3480); NME3(4832); TSC2(7249); TRAF7(84231); CCNF(899); TNFRSF12A(51330); |
| | ERN2(10595); P8(26471); PRRT2(112476); CYLD(1540); FTS(64400); LOC283849(283849); E2F4(1874); CTCF(10664); |
| | SLC12A4(6560); KIAA0513(9764); PCOLN3(5119); ABR(29); HIC1(3090); PAFAH1B1(5048); GARNL4(23108); |
| | TNFSF12(8742); TP53(7157); GAS7(8522); TOM1L2(146691); POLDIP2(26073); TRAF4(9618); NF1(4763); MLLT6(4302); |
| | PTRF(284119); BRCA1(672); TBKBP1(9755); IGF2BP1(10642); PHB(5245); NGFR(4804); CROP(51747); DYNLL2 |
| | (140735); MPO(4353); PRKCA(5578); CDK3(1018); SOCS3(9021); BAIAP2(10458); CABLES1(91768); RNF165(494470); |
| | KIAA0427(9811); BCL2(596); NFATC1(4772); SF3A2(8175); GNG7(2788); FZR1(51343); PIN1(5300); CDC37(11140); |
| | TNPO2(30000); LPHN1(22859); SIRT2(22933); IGSF4C(199731); FOSB(2354); DMWD(1762); BBC3(27113); RPS11 |
| | (6205); PPP2R1A(5518); GRLF1(2909); ZNF343(79175); C20orf116(65992); E2F1(1869); CEP250(11190); PPP1R16B |
| | (26051); ZHX3(23051); RIMS4(140730); STK4(6789); DIDO1(11083); TNFRSF6B(8771); ZGPAT(84619); APP |
| | (351); ADARB1(104); LSS(4047); HIC2(23119); MAPK1(5594); PPM1F(9647); MIF(4282); ADORA2A(135); NF2(4171); |
| | KIAA1904(114794); MGAT3(4248); ZC3H7B(23264); NHP2L1(4809); SEPT3(55964); GTSE1(51512); PCTK1(5127); IQSEC2 |
| | (23096); TMEM28(27112); MLLT7(4303); RP6-213H19.1(51765); CD40LG(959); HCFC1(3054); PRKY(5616); |
| 23 | ANKRD36(375248) |

TABLE 2-5

| | |
|---|---|
| 24 | NFIX(4784) |
| 25 | SDF4(51150); B3GALT6(126792); UBE2J2(118424); CENTB5(116983); SKI(6497); C1orf93(127281); SPSB1(80176); |
| | PIK3CD(5293); TINAGL1(64129); KIAA0319L(79932); IGSF4B(57863); ZNF648(127665); WNT3A(89780); OR2W5(441932); |
| | OR2C3(81472); ATAD3C(219293); INPP5D(3635); ANKRD23(200539); RAMP1(10267); RIS1(25907); PH-4(54681); |
| | IHPK1(9807); PLXNA1(5361); CLDN18(51208); LIPH(200879); GP5(2814); CPLX1(10815); MAEA(10296); RGS12 |
| | (6002); LOC93622(93622); DCAMKL2(166614); DUX4(22947); AHRR(57491); TPPP(11076); CTNND2(1501); PITX1 |
| | (5307); PCDHA1(56147); PCDHA2(56146); PCDHA3(56145); PCDHA4(56144); PCDHA5(56143); PCDHA6(56142); |
| | PCDHA7(56141); PCDHA8(56140); PCDHA9(9752); PCDHA10(56139); PCDHA11(56138); PCDHA12(56137); PCDHA13 |
| | (56136); PCDHAC1(56135); PCDHAC2(56134); SH3PXD2B(285590); UNC5A(90249); DBN1(1627); LOC51149(51149); |
| | C6orf85(63027); C6orf145(221749); TULP1(7287); COL12A1(1303); TCF21(6943); RPS6KA2(6196); VGF(7425); |
| | BCAP29(55973); EPHA1(2041); ATG9B(285973); LRCH4(4034); KIAA1688(80728); CDKN2B(1030); B4GALT1(2683); |
| | KLF9(687); IPPK(64768); DAB2IP(153090); BRD3(8019); PTGDS(5730); FLJ45224(401562); C9orf140(89958); MGC61598 |
| | (441478); LOC399706(399706); C10orf114(399726); LOC439985(439985); C10orf116(10974); FRAT1(10023); |
| | MARVELD1(83742); C10orf39(282973); STK32C(282974); INPP5A(3632); FBXO3(26273); CTTN(2017); DLG2(1740); |
| | DKFZp547C195(257160); KIRREL3(84623); HOM-TES-103(25900); DDN(23109); METTL7A(25840); HOXC13 |
| | (3229); CDK4(1019); IPF1(3651); C13orf21(387923); SLITRK5(26050); ATP11A(23250); GPR68(8111); PPP2R5C(5527); |
| | BRF1(2972); PACS2(23241); C15orf37(283687); UBE2I(7329); IFT140(9742); CASKIN1(57524); ZNF213(7760); ZNF500 |
| | (26048); SEPT1(1731); FBXL19(54620); GPT2(84706); MON1B(22879); MGC51025(353149); TRAF4(9618); ANKRD13B |
| | (124930); CCDC55(84081); RAB11FIP4(84440); LHX1(3975); PCGF2(7703); HLF(3131); KIAA0195(9772); C1QTNF1 |
| | (114897); HOXB8(3218); C18orf43(10650); RNF165(494470); KIAA0427(9811); CDH20(28316); TXNL4A(10907); |
| | WDR18(57418); MKNK2(2872); MOBKL2A(126308); LMNB2(84823); DAPK3(1613); TNFAIP8L1(126282); JMJD2B |
| | (23030); TIMM44(10469); RAVER1(125950); NFIX(4784); F2RL3(9002); ZBTB32(27033); RASGRP4(115727); CAPN12 |
| | (147968); PSCD2(9266); SPHK2(56848); BCAT2(587); LIN7B(64130); PRKCG(5582); LENG8(114823); LOC352909 |
| | (352909); ICAM4(3386); EEF2(1938); GRLF1(2909); UBOX5(22888); BLCAP(10904); SPATA2(9825); D21S2056E |
| | (8568); POFUT2(23275); SEPT5(5413); TBX1(6899); LOC150223(150223); BRD1(23774); EDA(1896); FAM11A(84548); |
| | CHM(1121); |
| 26 | ZFP41(286128) |
| 27 | PRDM16(63976) |
| 28 | CLASP1(23332) |
| 29 | ZNF655(79027) |
| 30 | FAIM2(23017) |
| 31 | ZGPAT(84619) |
| 32 | E2F2(1870); CCDC21(64793); LAPTM5(7805); ZBTB8(127557); MGC33556(339541); RAB3B(5865); CDC42SE1(56882); |
| | UHMK1(127933); IL24(11009); TRAF5(7188); C1orf95(375057); VANGL1(81839); SFXN5(94097); CASP10(843); |
| | CD28(940); COL4A3(1285); FLJ10996(54520); GRM7(2917); SRGAP3(9901); PH-4(54681); RASSF1(11186); PRICKLE2 |
| | (166336); ST3GAL6(10402); PCNP(57092); RNF7(9616); HIP2(3093); TMPRSS11E(28983); OSMR(9180); F2R(2149); |
| | TICAM2(353376); SEMA6A(57556); N4BP3(23138); ELOVL2(54898); IHPK3(117283); CCND3(896); HSP90AB1 |
| | (3326); PAQR8(85315); CCNC(892); ATG5(9474); FOXO3A(2309); SLC22A3(6581); ELMO1(9844); NUDCD3(23386); |
| | CALN1(83698); BCAP29(55973); MSR1(4481); DUSP4(1846); UNC5D(137970); TACC1(6867); UBE2V2(7336); YTHDF3 |
| | (253943); EXT1(2131); LRRC6(23639); ST3GAL1(6482); C8orf30A(51236); MGC21881(389741); KIAA1815(79956); |
| | RECK(8434); SEMA4D(10507); TNFSF8(944); C10orf9(219771); ARID5B(84159); SGPL1(8879); HIF1AN(55662); |
| | FAM26C(255022); C10orf137(26098); FLJ46154(196296); TRAF6(7189); ACAT1(38); SNF1LK2(23235); CDON(50937); |
| | KCNA6(3742); PTMS(5763); BCAT1(586); KRAS(3845); HDAC7A(51564); TEGT(7009); C12orf22(81566); ZBTB39 |
| | (9880); FLJ11259(55332); THRAP2(23389); RFP2(10206); STRN3(29966); C14orf118(55668); UBE3A(7337); KLF13 |
| | (51621); Gcom1(145781); THSD4(79875); GRIN2A(2903); CTCF(10664); MPHOSPH6(10200); POLDIP2(26073); SARM1 |
| | (23098); NF1(4763); EIF1(10209); TMEM101(84336); PPM1D(8493); HOXB9(3219); BCL2(596); PRX(57716); ERF |
| | (2077); GRLF1(2909); BTBD4(140685); PARD6B(84612); MAPK1(5594); CACNA1I(8911); TNRC6B(23112); SEPT3(55964); |
| | SSX4B(548313); PCYT1B(9468); SRPX(8406); OATL1(4943); SMC1L1(8243); SPRY3(10251); |
| 33 | ADCY1(107) |
| 34 | CNNM4(26504) |
| 35 | FUT3(2525) |
| 36 | RER1(11079); SASS6(163786); CDC14A(8556); RAP1A(5906); MCL1(4170); CKS1B(1163); F11R(50848); BRP44(25874); |
| | DNM3(26052); RGS2(5997); MFSD4(148808); PFKFB2(5208); PROX1(5629); CCNL2(81669); MSH2(4436); SERTAD2 |
| | (9792); EDAR(10913); ZC3H6(376940); WDR33(55339); MCM6(4175); CYBRD1(79901); PTD004(29789); STAT1 |
| | (6772); CASP10(843); CUL3(8452); HDAC4(9759); FLJ45964(401040); SRGAP3(9901); TMEM16K(55129); TDGF1( |

TABLE 2-6

| | 6997); APPL(26060); AER61(285203); PROK2(60675); CLDND1(56650); ACPP(55); CLSTN2(64084); XRN1(54464); SMC4L1 |
|---|---|
| | (10051); KLHL6(89857); YEATS2(55689); SENP2(59343); TP73L(8626); MGC21675(92070); HD(3064); DKFZp761B107 |
| | (91050); IL8(3576); FRAS1(80144); PRKG2(5593); DNAJB14(79962); TIFA(92610); MAD2L1(4085); FGF2 |
| | (2247); LOC134145(134145); PRLR(5618); LIFR(3977); PLCXD3(345557); MRPS30(10884); LOC153364(153364); APC |
| | (324); MCC(4163); SLC12A2(6558); FGF1(2246); G3BP(10146); GCNT2(2651); TCF19(6941); TNFRSF21(27242); ICK |
| | (22858); COL19A1(1310); CNR1(1268); HACE1(57531); BVES(11149); ATG5(9474); SESN1(27244); SERINC1(57515); |
| | HEY2(23493); TNFAIP3(7128); LOC90639(90639); TRIAD3(54476); LOC90693(90693); CYCS(54205); CREB5 |
| | (9586); CDK6(1021); NAPE-PLD(222236); RELN(5649); ZC3HAV1(56829); CNOT7(29883); MTMR7(9108); ADRA1A |
| | (148); UNC5D(137970); SPFH2(11160); BRF2(55290); UBE2V2(7336); UBE2W(55284); LRRCC1(85444); E2F5(1875); |
| | MTSS1(9788); GNAQ(2776); C9orf47(286223); CDC14B(8555); NR4A3(8013); C1QL3(389941); PARD3(56288); ZNF365 |
| | (22891); SGPL1(8879); COX15(1355); NOLC1(9221); GPR26(2849); EIF4G2(1982); DLG2(1740); CUL5(8065); PPP2R1B |
| | (5519); ARHGEF12(23365); BCL2L14(79370); ATF7IP(55729); CPNE8(144402); TMEM16F(196527); SP1(6667); |
| | EEA1(8411); APAF1(317); HCFC2(29915); FBXO21(23014); 15E1.2(283459); P2RX7(5027); TMEM132B(114795); |
| | ETNK1(55500); DIP13B(55198); LATS2(26524); TNFRSF19(55504); FLT1(2321); HSPH1(10808); FOXO1A(2308); SUGT1 |
| | (10910); RAP2A(5911); FLJ10769(55739); CUL4A(8451); METTL3(56339); KIAA1443(57594); PPM1A(5494); MAP3K9 |
| | (4293); C14orf43(91748); LTBP2(4053); DIO2(1734); ITPK1(3705); EIF5(1983); JAG2(3714); GOLGA8G(283768); |
| | TP53BP1(7158); FGF7(2252); NARG2(79664); FEM1B(10116); IREB2(3658); ABHD2(11057); GSPT1(2935); MKL2 |
| | (57496); GABARAPL2(11345); GAN(8139); CDH13(1012); MNT(4335); TRAF4(9618); BRCA1(672); NPEPPS(9520); |
| | MXRA7(439921); CARD14(79092); RAB12(201475); VAPA(9218); CEP192(55125); C18orf1(753); CTAGE1(64693); |
| | MALT1(10892); PHLPP(23239); CLEC4M(10332); GGTL3(2686); C20orf117(140710); RBL1(5933); UBE2V1(7335); CSTF1 |
| | (1477); CABLES2(81928); SON(6651); SLC5A3(6526); CDC45L(8318); MAPK1(5594); MN1(4330); DEPDC5(9681); |
| | MCM5(4174); TNRC6B(23112); LOC63929(63929); SEPT3(55964); ATXN10(25814); ZBED4(9889); PRKX(5613); |
| | AGTR2(186); SEPT6(23157); ATP1B4(23439); RP6-213H19.1(51765); FMR1(2332); DKC1(1736); AFF2(2334); |
| 37 | ZKSCAN1(7586) |
| 38 | TPPP(11076) |
| 39 | SRGAP3(9901) |
| 40 | DFFA(1676) |
| 41 | LIMD1(8994) |
| 42 | FSTL3(10272) |
| 43 | NUDCD3(23386) |
| 44 | GIPC3(126326) |
| 45 | IQCE(23288) |
| 46 | CMTM4(146223) |
| 47 | LETM1(3954) |
| 48 | GMFB(2764) |
| 49 | NFIX(4784) |
| 50 | PIK3R1(5295) |
| 51 | ATXN1(6310) |
| 52 | RP5-875H10.1(389432) |
| 53 | TPI1(7167) |
| 54 | ZNF192(7745) |
| 55 | ABO(28) |
| 56 | PRX(57716) |
| 57 | PALM2(114299) |
| 58 | NFIX(4784) |

TABLE 2-7

| | |
|---|---|
| 59 | RBMY1A1(5940) |
| 60 | TNFRSF8(943) |
| 61 | CRTC1(23373) |
| 62 | BBS5(129880) |
| 63 | COL27A1(85301) |
| 64 | DGCR14(8220) |
| 65 | EXOSC6(118460) |
| 66 | EIF2C4(192670) |
| 67 | RAB30(27314) |
| 68 | KCNMA1(3778) |
| 69 | CCND2(894) |
| 70 | ADARB2(105) |
| 71 | FYCO1(79443) |
| 72 | ADCY7(113) |
| 73 | IGSF4D(253559) |
| 74 | GNG13(51764) |
| 75 | ENTPD7(57089) |
| 76 | TMEM110(375346) |
| 77 | CREB3L3(84699) |
| 78 | ATXN1(6310) |
| 79 | ADARB2(105) |
| 80 | PRLR(5618) |
| 81 | ADARB2(105) |
| 82 | XAB1(11321) |
| 83 | MGAT4A(11320) |
| 84 | NBPF11(200030) |
| 85 | LIN10(80262) |
| 86 | SPOCK2(9806) |
| 87 | NTN1(9423) |
| 88 | ISG20L2(81875) |
| 89 | GGTL3(2686) |
| 90 | LRPAP1(4043) |
| 91 | GGTL3(2686) |
| 92 | IGF2(3481) |
| 93 | NEURL(9148) |
| 94 | TNRC6B(23112) |
| 95 | FN3K(64122) |
| 96 | BRWD1(54014) |
| 97 | DISC1(27185) |
| 98 | ENAH(55740) |
| 99 | GDF11(10220) |
| 100 | RAB22A(57403) |
| 101 | APOE(348) |
| 102 | PSD3(23362) |
| 103 | PSD3(23362) |
| 104 | NKTR(4820) |

TABLE 2-8

| | |
|---|---|
| 105 | JDP2(122953) |
| 106 | RAB40C(57799) |
| 107 | CNTN2(6900) |
| 108 | PRX(57716) |
| 109 | APOL6(80830) |
| 110 | FOXK1(221937) |
| 111 | YIPF6(286451) |
| 112 | HOXB8(3218) |
| 113 | CLN8(2055) |
| 114 | PRX(57716) |
| 115 | EVC(2121) |
| 116 | VANGL1(81839) |
| 117 | TNRC6B(23112) |
| 118 | JPH4(84502) |
| 119 | PHACTR2(9749) |
| 120 | LRRC27(80313) |
| 121 | TBL1X(6907) |
| 122 | BBS7(55212) |
| 123 | BDKRB2(624) |
| 124 | LPP(4026) |
| 125 | SAMD11(148398) |
| 126 | TNRC6B(23112) |
| 127 | ZGPAT(84619) |
| 128 | TNRC6B(23112) |
| 129 | RASSF5(83593) |
| 130 | USH1G(124590) |
| 131 | HBXAP(51773) |
| 132 | HIF3A(64344) |
| 133 | LRPAP1(4043) |
| 134 | DIAPH2(1730) |
| 135 | RAB8A(4218) |
| 136 | PRDM16(63976) |
| 137 | STX3A(6809) |
| 138 | AK1(203) |
| 139 | ENAH(55740) |
| 140 | PURB(5814) |
| 141 | MON1B(22879) |
| 142 | EVC(2121) |
| 143 | PQLC2(54896) |
| 144 | BRSK2(9024) |
| 145 | ATP11A(23250) |
| 146 | TSPAN14(81619) |
| 147 | CYP4F3(4051) |
| 148 | COPG2(26958) |
| 149 | PTPRT(11122) |
| 150 | ZNF84(7637) |

TABLE 2-10

| | |
|---|---|
| 151 | CACNA1A(773) |
| 152 | GIPC3(126326) |
| 153 | RAB36(9609) |
| 154 | DNM3(26052) |
| 155 | GABRA4(2557) |
| 156 | FOXK1(221937) |
| 157 | LAT(27040) |
| 158 | ABHD2(11057) |
| 159 | ENTPD7(57089) |
| 160 | ATP11A(23250) |
| 161 | CLCA1(1179) |
| 162 | MARCH3(115123) |
| 163 | USH1G(124590) |
| 164 | FOXK1(221937) |
| 165 | PRSS16(10279) |
| 166 | BLR1(643) |
| 167 | ANKRD30B(374860) |
| 168 | DCX(1641) |
| 169 | CACNA1A(773) |
| 170 | KATNAL1(84056) |
| 171 | PRTG(283659) |
| 172 | ADCY7(113) |
| 173 | EIF4EBP2(1979) |
| 174 | HEXDC(284004) |
| 175 | H6PD(9563) |
| 176 | APC2(10297) |
| 177 | SF4(57794) |
| 178 | BBS5(129880) |
| 179 | TBC1D16(125058) |
| 180 | CHD5(26038) |
| 181 | CBX5(23468) |
| 182 | TGOLN2(10618) |
| 183 | ICMT(23463) |
| 184 | SELI(85465) |
| 185 | WASF2(10163) |
| 186 | TMC6(11322) |
| 187 | MUC17(140453) |
| 188 | ADCY1(107) |
| 189 | UNQ6125(442092) |
| 190 | LRRC27(80313) |
| 191 | RAB30(27314) |
| 192 | CBL(867) |
| 193 | GOLGA3(2802) |
| 194 | PTGFR(5737) |
| 195 | CDK6(1021) |
| 196 | PURB(5814) |

TABLE 2-11

| | |
|---|---|
| 197 | PTGFR(5737) |
| 198 | FUT4(2526) |
| 199 | LYNX1(66004) |
| 200 | SNX8(29886) |
| 201 | CDK6(1021) |
| 202 | PRKAA2(5563) |
| 203 | ADCY1(107) |
| 204 | IPO9(55705) |
| 205 | TBL3(10607) |
| 206 | MLLT6(4302) |
| 207 | ANKRD30B(374860) |
| 208 | MAP6(4135) |
| 209 | CLN8(2055) |
| 210 | GRM4(2914) |
| 211 | NTRK2(4915) |
| 212 | REXO1L1(254958) |
| 213 | EPB41L5(57669) |
| 214 | TMED4(222068) |
| 215 | ERBB4(2066) |
| 216 | RIPK5(25778) |
| 217 | APOE(348) |
| 218 | BOK(666) |
| 219 | BAG5(9529) |
| 220 | TRAF7(84231) |
| 221 | LRRC27(80313) |
| 222 | HOXB8(3218) |
| 223 | PRKAA2(5563) |
| 224 | RAB40C(57799) |
| 225 | AFF4(27125) |
| 226 | GIPC3(126326) |
| 227 | H-plk(51351) |
| 228 | PIK3CA(5290) |
| 229 | LRRC14(9684) |
| 230 | NAV1(89796) |
| 231 | PRTG(283659) |
| 232 | SPRY3(10251) |
| 233 | PDE4D(5144) |
| 234 | ZNF440L(284390) |
| 235 | PSD3(23362) |
| 236 | GABRA4(2557) |
| 237 | ADCY1(107) |
| 238 | F7(2155) |
| 239 | KLF7(8609) |
| 240 | RP6-166C19.1(255313) |
| 241 | NFAT5(10725) |
| 242 | PRTG(283659) |

TABLE 2-12

| | |
|---|---|
| 243 | SAMD11(148398) |
| 244 | ZGPAT(84619) |
| 245 | RAB40C(57799) |
| 246 | GRIN2A(2903) |
| 247 | LIMD1(8994) |
| 248 | TNRC6B(23112) |
| 249 | GIPC3(126326) |
| 250 | GABRA4(2557) |
| 251 | TNRC6B(23112) |
| 252 | TMED4(222068) |
| 253 | MXRA7(439921) |
| 254 | AFG3L1(172) |
| 255 | TTYH3(80727) |
| 256 | DCP2(167227) |
| 257 | QKI(9444) |
| 258 | NFIX(4784) |
| 259 | ENAH(55740) |
| 260 | WDR37(22884) |
| 261 | NUDT3(11165) |
| 262 | KBTBD8(84541) |
| 263 | CD59(966) |
| 264 | UNQ6125(442092) |
| 265 | SH3PXD2A(9644) |
| 266 | FOXK1(221937) |
| 267 | CREB3L2(64764) |
| 268 | HOXB8(3218) |
| 269 | YPEL1(29799) |
| 270 | PRX(57716) |
| 271 | CXorf15(55787) |
| 272 | FOXK1(221937) |
| 273 | GIPC3(126326) |
| 274 | SCLY(51540) |
| 275 | RP11-114H20.1(401589) |
| 276 | SMU1(55234) |
| 277 | CALML4(91860) |
| 278 | SNX8(29886) |
| 279 | TP73L(8626) |
| 280 | ENTPD7(57089) |
| 281 | AFG3L1(172) |
| 282 | RAPH1(65059) |
| 283 | SLC36A1(206358) |
| 284 | RBJ(51277) |
| 285 | MUCDHL(53841) |
| 286 | KRTHB5(3891) |
| 287 | SEMA3E(9723) |
| 288 | WDR35(57539) |

TABLE 2-13

| | |
|---|---|
| 289 | SIPA1L3(23094) |
| 290 | GGTL3(2686) |
| 291 | PCYT2(5833) |
| 292 | ENAH(55740) |
| 293 | ANK1(286) |
| 294 | GRIA1(2890) |
| 295 | GIPC3(126326) |
| 296 | VAPB(9217) |
| 297 | PDPR(55066) |
| 298 | BRCC3(79184) |
| 299 | LETM1(3954) |
| 300 | SARM1(23098) |
| 301 | DCP2(167227) |
| 302 | TAGLN(6876) |
| 303 | TPPP(11076) |
| 304 | SEC14L1(6397) |
| 305 | H-plk(51351) |
| 306 | GIPC3(126326) |
| 307 | HS6ST1(9394) |
| 308 | EVC(2121) |
| 309 | SLC6A3(6531) |
| 310 | SCRT1(83482) |
| 311 | RP11-114H20.1(401589) |
| 312 | JPH4(84502) |
| 313 | CREB3L3(84699) |
| 314 | BOK(666) |
| 315 | ZFPL(162967) |
| 316 | POU3F1(5453) |
| 317 | GM632(57473) |
| 318 | ZNF213(7760) |
| 319 | PHF15(23338) |
| 320 | PRX(57716) |
| 321 | TRAF6(7189) |
| 322 | ANKRD36(375248) |
| 323 | NFATC1(4772) |
| 324 | FBXO40(51725) |
| 325 | ZGPAT(84619) |
| 326 | PDXK(8566) |
| 327 | SFRS8(6433) |
| 328 | CACNA1I(8911) |
| 329 | AGPAT4(56895) |
| 330 | EVC(2121) |
| 331 | JARID1A(5927) |
| 332 | ZNF641(121274) |
| 333 | PGAP1(80055) |
| 334 | DSC2(1824) |

TABLE 2-14

| | |
|---|---|
| 335 | ABHD2(11057) |
| 336 | BDKRB2(624) |
| 337 | BCAM(4059) |
| 338 | SCYL1(57410) |
| 339 | MARCH1(55016) |
| 340 | USP31(57478) |
| 341 | GDF6(392255) |
| 342 | BCL2(596) |
| 343 | DKFZP434B0335(25851) |
| 344 | PLCXD3(345557) |
| 345 | IGF2(3481) |
| 346 | MTHFR(4524) |
| 347 | WDR33(55339) |
| 348 | KCNA7(3743) |
| 349 | ADCY1(107) |
| 350 | CHD5(26038) |
| 351 | AFF2(2334) |
| 352 | SPN(6693) |
| 353 | JARID1A(5927) |
| 354 | HIF3A(64344) |
| 355 | TMC6(11322) |
| 356 | ENTPD7(57089) |
| 357 | LTB4R2(56413) |
| 358 | ADARB2(105) |
| 359 | TRO(7216) |
| 360 | ASB1(51665) |
| 361 | MAPK1(5594) |
| 362 | NFAT5(10725) |
| 363 | EEA1(8411) |
| 364 | ZNF705CP(389631) |
| 365 | CREB5(9586) |
| 366 | EXOSC6(118460) |
| 367 | FMNL3(91010) |
| 368 | LETM1(3954) |
| 369 | GGTL3(2686) |
| 370 | CDK6(1021) |
| 371 | SBK1(388228) |
| 372 | EIF4EBP2(1979) |
| 373 | OPCML(4978) |
| 374 | MECP2(4204) |
| 375 | TNFRSF13B(23495) |
| 376 | TNRC6B(23112) |
| 377 | STX7(8417) |
| 378 | CREB1(1385) |
| 379 | CBX5(23468) |
| 380 | NFIX(4784) |

TABLE 2-15

| | |
|---|---|
| 381 | ADARB2(105) |
| 382 | TNS1(7145) |
| 383 | SELI(85465) |
| 384 | LETM1(3954) |
| 385 | FGFRL1(53834) |
| 386 | ZNF440L(284390) |
| 387 | GABRG1(2565) |
| 388 | DECR2(26063) |
| 389 | HTRA2(27429) |
| 390 | LPAL2(80350) |
| 391 | OPCML(4978) |
| 392 | USP37(57695) |
| 393 | ITR(160897) |
| 394 | SFRS8(6433) |
| 395 | APOE(348) |
| 396 | FGD3(89846) |
| 397 | LRRC27(80313) |
| 398 | H6PD(9563) |
| 399 | ADAM22(53616) |
| 400 | LRRC27(80313) |
| 401 | CREB3L3(84699) |
| 402 | OXCT2(64064) |
| 403 | DISC1(27185) |
| 404 | NEURL(9148) |
| 405 | WDR55(54853) |
| 406 | FAM8A1(51439) |
| 407 | GABRB2(2561) |
| 408 | NELF(26012) |
| 409 | H6PD(9563) |
| 410 | TNFRSF13B(23495) |
| 411 | WNT7B(7477) |
| 412 | ADARB2(105) |
| 413 | CXorf6(10046) |
| 414 | TPPP(11076) |
| 415 | DGCR14(8220) |
| 416 | PKN3(29941) |
| 417 | LRPAP1(4043) |
| 418 | CER1(9350) |
| 419 | VGLL3(389136) |
| 420 | PCDHGB4(8641) |
| 421 | RAB40C(57799) |
| 422 | YOD1(55432) |
| 423 | CREB1(1385) |
| 424 | SLC7A1(6541) |
| 425 | CHTF18(63922) |
| 426 | CALN1(83698) |

TABLE 2-16

| | |
|---|---|
| 427 | BSDC1(55108) |
| 428 | NUAK1(9891) |
| 429 | GIPC3(126326) |
| 430 | RASGEF1B(153020) |
| 431 | MAGI1(9223) |
| 432 | BSDC1(55108) |
| 433 | VGLL3(389136) |
| 434 | NF2(4771) |
| 435 | PDXK(8566) |
| 436 | GABRG1(2565) |
| 437 | LIMD1(8994) |
| 438 | L3MBTL2(83746) |
| 439 | TPPP(11076) |
| 440 | PRKAA2(5563) |
| 441 | BRWD1(54014) |
| 442 | SELI(85465) |
| 443 | LRRC27(80313) |
| 444 | CALN1(83698) |
| 445 | GATAD2A(54815) |
| 446 | SH3BP2(6452) |
| 447 | CDR1(1038) |
| 448 | POU3F3(5455) |
| 449 | TMEM56(148534) |
| 450 | CDR1(1038) |
| 451 | ASB1(51665) |
| 452 | GPIAP1(4076) |
| 453 | VAV2(7410) |
| 454 | JARID1A(5927) |
| 455 | TRIM14(9830) |
| 456 | TPPP(11076) |
| 457 | BDKRB2(624) |
| 458 | E2F2(1870) |
| 459 | APBB3(10307) |
| 460 | LRPAP1(4043) |
| 461 | RGPD2(440872) |
| 462 | PALM2(114299) |
| 463 | APPL(26060) |
| 464 | TPPP(11076) |
| 465 | GGTL3(2686) |
| 466 | RAB40C(57799) |
| 467 | PRMT8(56341) |
| 468 | POFUT2(23275) |
| 469 | STK38L(23012) |
| 470 | BOK(666) |
| 471 | CRTC1(23373) |
| 472 | FAM20B(9917) |

TABLE 2-17

| | |
|---|---|
| 473 | ICMT(23463) |
| 474 | BAK1(578) |
| 475 | SLC36A4(120103) |
| 476 | SEPT5(5413) |
| 477 | EVC(2121) |
| 478 | PRTG(283659) |
| 479 | TRIM33(51592) |
| 480 | ZFP41(286128) |
| 481 | PDXK(8566) |
| 482 | TEAD1(7003) |
| 483 | ENAH(55740) |
| 484 | PLXDC1(57125) |
| 485 | PRX(57716) |
| 486 | AGPAT3(56894) |
| 487 | RAB40C(57799) |
| 488 | LRPAP1(4043) |
| 489 | PRELP(5549) |
| 490 | ZNF44(51710) |
| 491 | GNG13(51764) |
| 492 | PRKAA2(5563) |
| 493 | PRTG(283659) |
| 494 | GABRA4(2557) |
| 495 | CRTAP(10491) |
| 496 | PSD3(23362) |
| 497 | BCAT1(586) |
| 498 | RP11-308D16.4(441522) |
| 499 | LRPAP1(4043) |
| 500 | PFKFB2(5208) |
| 501 | PPM1F(9647) |
| 502 | GPR123(84435) |
| 503 | FMNL3(91010) |
| 504 | PRTG(283659) |
| 505 | GRIN2A(2903) |
| 506 | POU3F3(5455) |
| 507 | SNX27(81609) |
| 508 | BOK(666) |
| 509 | RAB8A(4218) |
| 510 | TNRC6B(23112) |
| 511 | ERBB4(2066) |
| 512 | GABRA4(2557) |
| 513 | ASB1(51665) |
| 514 | MECP2(4204) |
| 515 | ZNF264(9422) |
| 516 | ASB1(51665) |
| 517 | RBMS2(5939) |
| 518 | GRIN2A(2903) |

TABLE 2-18

| | |
|---|---|
| 519 | SOX11(6664) |
| 520 | KLF12(11278) |
| 521 | CBL(867) |
| 522 | PGAP1(80055) |
| 523 | DDEF2(8853) |
| 524 | MINA(84864) |
| 525 | TNRC6B(23112) |
| 526 | LRPAP1(4043) |
| 527 | AXL(558) |
| 528 | ZNF289(84364) |
| 529 | NEURL(9148) |
| 530 | FZD4(8322) |
| 531 | PRLR(5618) |
| 532 | PB1(55193) |
| 533 | ARF3(377) |
| 534 | JARID1A(5927) |
| 535 | POLR2J2(246721) |
| 536 | SYNCRIP(10492) |
| 537 | PAG1(55824) |
| 538 | EXOC5(10640) |
| 539 | DENR(8562) |
| 540 | RAB40C(57799) |
| 541 | RNF165(494470) |
| 542 | SLC38A5(92745) |
| 543 | FOXK1(221937) |
| 544 | SOCS7(30837) |
| 545 | RNF40(9810) |
| 546 | LRPAO1(4043) |
| 547 | STX7(8417) |
| 548 | EVC(2121) |
| 549 | ERGIC1(57222) |
| 550 | LPAL2(80350) |
| 551 | DDR2(4921) |
| 552 | ENAH(55740) |
| 553 | ENAH(55740) |
| 554 | FN3K(64122) |
| 555 | ZNF214(7760) |
| 556 | LIMD1(8994) |
| 557 | SNPH(9751) |
| 558 | PRX(57716) |
| 559 | SAMD11(148398) |
| 560 | FREQ(23413) |
| 561 | HNRPU(3192) |
| 562 | TMC6(11322) |
| 563 | RAB40C(57799) |
| 564 | WDR37(22884) |

TABLE 2-19

| | |
|---|---|
| 565 | EGFR(1956) |
| 566 | FAM9C(171484) |
| 567 | TMED4(222068) |
| 568 | FAM102B(284611) |
| 569 | GNG4(2786) |
| 570 | RP6-166C19.1(255313) |
| 571 | KSR1(8844) |
| 572 | DZIP1(22873) |
| 573 | TPPP(11076) |
| 574 | RP6-166C19.1(255313) |
| 575 | ISG20L2(81875) |
| 576 | BMPR2(659) |
| 577 | ATP9A(10079) |
| 578 | GAS7(8522) |
| 579 | ENAH(55740) |
| 580 | BTRC(8945) |
| 581 | SUPT3H(8464) |
| 582 | RPS15A(6210) |
| 583 | GATAD1(57798) |
| 584 | FOXK1(221937) |
| 585 | AP3S2(10239) |
| 586 | HEMK1(51409) |
| 587 | ARNT2(9915) |
| 588 | H6PD(9563) |
| 589 | APOE(348) |
| 590 | PRX(57716) |
| 591 | LETM1(3954) |
| 592 | CRAMP1L(57585) |
| 593 | LTB4R2(56413) |
| 594 | RASSF2(9770) |
| 595 | TPPP(11076) |
| 596 | ARGFX(503582) |
| 597 | SRGAP3(9901) |
| 598 | DUSP8(1850) |
| 599 | PRX(57716) |
| 600 | SEPT5(5413) |
| 601 | RBMS3(27303) |
| 602 | MECP2(4204) |
| 603 | SORD(6652) |
| 604 | ARFGAP1(55738) |
| 605 | GIPC3(126326) |
| 606 | MGAT2(4247) |
| 607 | ITPK1(3705) |
| 608 | SPFH2(11160) |
| 609 | TNRC6B(23112) |
| 610 | CREB1(1385) |

TABLE 2-20

| | |
|---|---|
| 611 | HOXB8(3218) |
| 612 | GGTL3(2686) |
| 613 | RBMS3(27303) |
| 614 | PSD3(23362) |
| 615 | BACH2(60468) |
| 616 | PIP5K1C(23396) |
| 617 | MTHFR(4524) |
| 618 | RBM14(10432) |
| 619 | LRRC27(80313) |
| 620 | APC2(10297) |
| 621 | LRRC14(9684) |
| 622 | PTGFR(5737) |
| 623 | TBC1D16(125058) |
| 624 | Ells1(222166) |
| 625 | ILF3(3609) |
| 626 | TNRC6B(23112) |
| 627 | RBM3(5935) |
| 628 | HCN2(610) |
| 629 | NT5DC2(64943) |
| 630 | MAPK1(5594) |
| 631 | PRX(57716) |
| 632 | AK2(204) |
| 633 | MTHFR(4524) |
| 634 | SEC24C(9632) |
| 635 | ING5(84289) |
| 636 | SCN5A(6331) |
| 637 | PTGDS(5730) |
| 638 | ZXDC(79364) |
| 639 | HIP1(3092) |
| 640 | FGFRL1(53834) |
| 641 | BIRC4(331) |
| 642 | FAIM2(23017) |
| 643 | CREB3L2(64764) |
| 644 | PAX2(5076) |
| 645 | GLS2(27165) |
| 646 | PCYT2(5833) |
| 647 | SRGAP3(9901) |
| 648 | GIPC3(126326) |
| 649 | PRRT2(112476) |
| 650 | SCRT1(83482) |
| 651 | GIPC3(126326) |
| 652 | SCRT1(83482) |
| 653 | ZYG11B(79699) |
| 654 | DRCTNNB1A(84668) |
| 655 | GGTL3(2686) |
| 656 | TPPP(11076) |

TABLE 2-21

| | |
|---|---|
| 657 | FSTL4(23105) |
| 658 | EVC(2121) |
| 659 | BRCA1(672) |
| 660 | AXL(558) |
| 661 | H-plk(51351) |
| 662 | TMEM10(93377) |
| 663 | DISC1(27185) |
| 664 | RPE(6120) |
| 665 | EVC(2121) |
| 666 | TPPP(11076) |
| 667 | FDPS(2224) |
| 668 | ZNF721(170960) |
| 669 | WNK3(65267) |
| 670 | SLC6A3(6531) |
| 671 | RBMS3(27303) |
| 672 | TRIM14(9830) |
| 673 | CBX5(23468) |
| 674 | ZNF493(284443) |
| 675 | GABRA4(2557) |
| 676 | CACNB4(785) |
| 677 | GPR123(84435) |
| 678 | TMEM132B(114795) |
| 679 | PMS2L2(5380) |
| 680 | KLF13(51621) |
| 681 | DAPK3(1613) |
| 682 | STK17A(9263) |
| 683 | SNX8(29886) |
| 684 | KCNMA1(3778) |
| 685 | GIPC3(126326) |
| 686 | AFG3L1(172) |
| 687 | TUSC5(286753) |
| 688 | TPPP(11076) |
| 689 | FN3K(64122) |
| 690 | WARS2(10352) |
| 691 | CSNK1G1(53944) |
| 692 | SH3BP2(6452) |
| 693 | FMNL3(91010) |
| 694 | GM632(57473) |
| 695 | PALM2(114299) |
| 696 | ZFP41(286128) |
| 697 | FOXK1(221937) |
| 698 | PRKAA2(5563) |
| 699 | LRRC27(80313) |
| 700 | ZF(58487) |
| 701 | BRCA1(672) |
| 702 | MGAT4A(11320) |

TABLE 2-22

| | |
|---|---|
| 703 | TRIM33(51592) |
| 704 | LTBP3(4054) |
| 705 | CUGBP2(10659) |
| 706 | ZNF440L(284390) |
| 707 | ISG20L1(64782) |
| 708 | BDKRB2(624) |
| 709 | ALPK3(57538) |
| 710 | TFRC(7037) |
| 711 | PCYT2(5833) |
| 712 | YEATS2(55689) |
| 713 | FDPS(2224) |
| 714 | BOK(666) |
| 715 | BBS5(129880) |
| 716 | ZBTB7A(51341) |
| 717 | TUBB1(81027) |
| 718 | DIDO1(11083) |
| 719 | SEC24C(9632) |
| 720 | GNG13(51764) |
| 721 | SAMD11(148398) |
| 722 | P2RXL1(9127) |
| 723 | SNN(8303) |
| 724 | GRM4(2914) |
| 725 | HD(3064) |
| 726 | BRCA1(672) |
| 727 | PCYT2(5833) |
| 728 | FAM26C(255022) |
| 729 | BOK(666) |
| 730 | MMP24(10893) |
| 731 | ADARB2(105) |
| 732 | TTYH3(80727) |
| 733 | SYT2(127833) |
| 734 | TTBK1(84630) |
| 735 | STK16(8576) |
| 736 | LRPAP1(4043) |
| 737 | TNFRSF13B(23495) |
| 738 | SNX8(29886) |
| 739 | SPN(6693) |
| 740 | PAG1(55824) |
| 741 | CNTN2(6900) |
| 742 | BRCA1(672) |
| 743 | SOX1(6656) |
| 744 | EML3(256364) |
| 745 | TENC1(23371) |
| 746 | GIPC3(126326) |
| 747 | CYCS(54205) |
| 748 | ZFP41(286128) |

TABLE 2-23

| | |
|---|---|
| 749 | JMJD2B(23030) |
| 750 | ZDHHC11(79844) |
| 751 | SNX8(29886) |
| 752 | ZNF721(170960) |
| 753 | LETM1(3954) |
| 754 | LETM1(3954) |
| 755 | NUFIP2(57532) |
| 756 | CBX5(23468) |
| 757 | CDK6(1021) |
| 758 | LRRC27(80313) |
| 759 | TCF2(6928) |
| 760 | ZNF24(7572) |
| 761 | SNX27(81609) |
| 762 | ZNF468(90333) |
| 763 | HIST2H4(8370) |
| 764 | VKORC1L1(154807) |
| 765 | ABHD2(11057) |
| 766 | ZNF226(7769) |
| 767 | TSPAN18(90139) |
| 768 | MCFD2(90411) |
| 769 | FDPS(2224) |
| 770 | TNRC6B(23112) |
| 771 | FGD3(89846) |
| 772 | BOK(666) |
| 773 | STAC2(342667) |
| 774 | RAB40C(57799) |
| 775 | SAPS3(55291) |
| 776 | BCAM(4059) |
| 777 | LEPR(3953) |
| 778 | RNF12(51132) |
| 779 | MECP2(4204) |
| 780 | MAPK1(5594) |
| 781 | MAP2K1IP1(8649) |
| 782 | FAM105B(90268) |
| 783 | TMC6(11322) |
| 784 | CBX5(23468) |
| 785 | LETM1(3954) |
| 786 | N4BP3(23138) |
| 787 | POU2AF1(5450) |
| 788 | NISCH(11188) |
| 789 | GPR123(84435) |
| 790 | EVC(2121) |
| 791 | ENAH(55740) |
| 792 | TMC6(11322) |
| 793 | BBS7(55212) |
| 794 | RAB40C(57799) |

TABLE 2-24

| | |
|---|---|
| 795 | SLC5A3(6526) |
| 796 | RNF31(55072) |
| 797 | LIFR(3977) |
| 798 | DIP(23151) |
| 799 | LEPR(3953) |
| 800 | DCP2(167227) |
| 801 | HIF3A(64344) |
| 802 | RFP2(10206) |
| 803 | JM11(90060) |
| 804 | BOK(666) |
| 805 | PRX(57716) |
| 806 | TPPP(11076) |
| 807 | PRX(57716) |
| 808 | JARID1A(5927) |
| 809 | PAPD1(55149) |
| 810 | FGFRL1(53834) |
| 811 | VAPB(9217) |
| 812 | RBM33(155435) |
| 813 | TBL3(10607) |
| 814 | GABRA4(2557) |
| 815 | ZNF721(170960) |
| 816 | PRDM10(56980) |
| 817 | APOE(348) |
| 818 | NEURL(9148) |
| 819 | KPNA1(3836) |
| 820 | LIFR(3977) |
| 821 | RAB40C(57799) |
| 822 | GRIN2A(2903) |
| 823 | FGF2(2247) |
| 824 | DIO2(1734) |
| 825 | SLC30A7(148867) |
| 826 | SNX27(81609) |
| 827 | EVI5(7813) |
| 828 | LRRC27(80313) |
| 829 | PPCDC(60490) |
| 830 | ITGAM(3684) |
| 831 | RNF12(51132) |
| 832 | TSPAN18(90139) |
| 833 | LRRC14(9684) |
| 834 | ADCY1(107) |
| 835 | PAG1(55824) |
| 836 | SOX11(6664) |
| 837 | AFF2(2334) |
| 838 | FSTL3(10272) |
| 839 | DISC1(27185) |
| 840 | ZDHHC2(51201) |

TABLE 2-25

| | |
|---|---|
| 841 | FSTL3(10272) |
| 842 | PAG1(55824) |
| 843 | MXRA7(439921) |
| 844 | SBK1(388228) |
| 845 | APOL6(80830) |
| 846 | GAB2(9846) |
| 847 | GLS2(27165) |
| 848 | SLC7A11(23657) |
| 849 | AFF2(2334) |
| 850 | MOBKL2B(79817) |
| 851 | PSD3(23362) |
| 852 | CHML(1122) |
| 853 | TNRC6B(23112) |
| 854 | NAPE-PLD(222236) |
| 855 | SOX5(6660) |
| 856 | APOL6(80830) |
| 857 | PDXK(8566) |
| 858 | FDPS(2224) |
| 859 | CBX5(23468) |
| 860 | CBX5(23468) |
| 861 | EIF4E3(317649) |
| 862 | SNX8(29886) |
| 863 | NKTR(4820) |
| 864 | SH3BP2(6452) |
| 865 | RAB40C(57799) |
| 866 | BAIAP2(10458) |
| 867 | RAB40C(57799) |
| 868 | CALML4(91860) |
| 869 | BRWD1(54014) |
| 870 | POU3F3(5455) |
| 871 | RBBP4(5928) |
| 872 | STK35(140901) |
| 873 | FMNL3(91010) |
| 874 | SNX1(6642) |
| 875 | SNX8(29886) |
| 876 | SGCD(6444) |
| 877 | TRIM14(9830) |
| 878 | FBXO28(23219) |
| 879 | QKI(9444) |
| 880 | PRR11(55771) |
| 881 | XPO4(64328) |
| 882 | LIMD1(8994) |
| 883 | PNMA5(114824) |
| 884 | MECP2(4204) |
| 885 | TNRC6B(23112) |
| 886 | SLC5A3(6526) |

TABLE 2-26

| | |
|---|---|
| 887 | GABRA4(2557) |
| 888 | SLC1A2(6506) |
| 889 | IHPK1(9807) |
| 890 | CD93(22918) |
| 891 | PGAP1(80055) |
| 892 | DCP2(167227) |
| 893 | SELI(85465) |
| 894 | GGTL3(2686) |
| 895 | CBX5(23468) |
| 896 | LIMD1(8994) |
| 897 | PARP11(57097) |
| 898 | TNRC6B(23112) |
| 899 | H6PD(9563) |
| 900 | BRWD1(54014) |
| 901 | SLC7A11(23657) |
| 902 | TMEM110(375346) |
| 903 | PRX(57716) |
| 904 | TBC1D16(125058) |
| 905 | POU3F3(5455) |
| 906 | SPN(6693) |
| 907 | NKTR(4820) |
| 908 | GABRA4(2557) |
| 909 | PNPO(55163) |
| 910 | TFB1M(51106) |
| 911 | WNK3(65267) |
| 912 | FOXK1(221937) |
| 913 | PLCXD3(345557) |
| 914 | PODXL(5420) |
| 915 | CBX5(23468) |
| 916 | CDK6(1021) |
| 917 | FSTL3(10272) |
| 918 | NEURL(9148) |
| 919 | LRPAP1(4043) |
| 920 | LETM1(3954) |
| 921 | NKTR(4820) |
| 922 | VANGL1(81839) |
| 923 | TNRC6B(23112) |
| 924 | H6PD(9563) |
| 925 | LRPAP1(4043) |
| 926 | ANKRD36(375248) |
| 927 | SEC22L3(9117) |
| 928 | RGPD5(84220) |
| 929 | CBX5(23468) |
| 930 | PACS1(55690) |
| 931 | MECP2(4204) |
| 932 | SLC1A2(6506) |

TABLE 2-27

| | |
|---|---|
| 933 | BDKRB2(624) |
| 934 | RAB40C(57799) |
| 935 | CBX5(23468) |
| 936 | SYNCRIP(10492) |
| 937 | GIPC3(126326) |
| 938 | TNRC6B(23112) |
| 939 | GOLGA3(2802) |
| 940 | LRRC27(80313) |
| 941 | HRB(3267) |
| 942 | WHSC1(7468) |
| 943 | RPE(6120) |
| 944 | RBL1(5933) |
| 945 | ST6GAL2(84620) |
| 946 | AFF3(3899) |
| 947 | BMPR2(659) |
| 948 | KPNA1(3836) |
| 949 | G3BP(10146) |
| 950 | G3BP(10146) |
| 951 | LRRC27(80313) |
| 952 | RP6-166C19.1(255313) |
| 953 | ZNF213(7760) |
| 954 | MBD3(53615) |
| 955 | NUDT3(11165) |
| 956 | TMC6(11322) |
| 957 | LRPAP1(4043) |
| 958 | ZNF721(170960) |
| 959 | RP6-166C19.1(255313) |
| 960 | DCP2(167227) |
| 961 | SARM1(23098) |
| 962 | ZNF510(22869) |
| 963 | CACNA2D2(9254) |
| 964 | JARID1A(5927) |
| 965 | CER1(9350) |
| 966 | SH3GLB2(56904) |
| 967 | ILF3(3609) |
| 968 | SYNGAP1(8831) |
| 969 | PEX19(5824) |
| 970 | LUZP1(7798) |
| 971 | LRRC27(80313) |
| 972 | GRHL2(79977) |
| 973 | FAM53A(152877) |
| 974 | HOXB8(3218) |
| 975 | GRIN2A(2903) |
| 976 | KIF13B(23303) |
| 977 | RAI1(10743) |
| 978 | ADCY1(107) |

TABLE 2-28

| | |
|---|---|
| 979 | FGD3(89846) |
| 980 | BCR(613) |
| 981 | RIMS3(9783) |
| 982 | FAM53A(152877) |
| 983 | TMED4(222068) |
| 984 | SLA(6503) |
| 985 | TMTC1(83857) |
| 986 | ARL5B(221079) |
| 987 | PERQ1(64599) |
| 988 | FADS1(3992) |
| 989 | TRAM2(9697) |
| 990 | NRCAM(4897) |
| 991 | BIRC4BP(54739) |
| 992 | IQCE(23288) |
| 993 | SYNJ2BP(55333) |
| 994 | HIF3A(64344) |
| 995 | RBMS2(5939) |
| 996 | ABC1(63897) |
| 997 | ANGEL2(90806) |
| 998 | PHOX2B(8929) |
| 999 | SLC6A3(6531) |
| 1000 | ZNF490(57474) |
| 1001 | ASB1(51665) |
| 1002 | NAV1(89796) |
| 1003 | KLK4(9622) |
| 1004 | NRXN3(9369) |
| 1005 | CIITA(4261) |
| 1006 | DLGAP2(9228) |
| 1007 | LRRC27(80313) |
| 1008 | LRRC27(80313) |
| 1009 | PURA(5813) |
| 1010 | ILF3(3609) |
| 1011 | ADAM22(53616) |
| 1012 | KATNAL1(84056) |
| 1013 | GOSR1(9527) |
| 1014 | DLEC1(9940) |
| 1015 | GALNTL2(117248) |
| 1016 | CER1(9350) |
| 1017 | SBF1(6305) |
| 1018 | RNF165(494470) |
| 1019 | FOXK1(221937) |
| 1020 | LRRC27(80313) |
| 1021 | TBC1D16(125058) |
| 1022 | FBXO42(54455) |
| 1023 | SYT8(90019) |
| 1024 | CALD1(800) |

TABLE 2-29

| | |
|---|---|
| 1025 | TMEM63A(9725) |
| 1026 | MAPK1(5594) |
| 1027 | PPP1R12B(4660) |
| 1028 | LETM1(3954) |
| 1029 | PARVA(55742) |
| 1030 | ELAVL1(1994) |
| 1031 | PSCD3(9265) |
| 1032 | WHSC1(7468) |
| 1033 | TCL1A(8115) |
| 1034 | SEPT5(5413) |
| 1035 | APBB3(10307) |
| 1036 | ADM2(79924) |
| 1037 | TMEM132B(114795) |
| 1038 | ATXN1(6310) |
| 1039 | LRPAP1(4043) |
| 1040 | PALM2(114299) |
| 1041 | TBC1D16(125058) |
| 1042 | LAMP1(3916) |
| 1043 | GRIN1(2902) |
| 1044 | PLXNA1(5361) |
| 1045 | VGLL3(389136) |
| 1046 | TRAF3(7187) |
| 1047 | ZFP41(286128) |
| 1048 | CREB3L3(84699) |
| 1049 | ERBB4(2066) |
| 1050 | NAV2(89797) |
| 1051 | PDLIM7(9260) |
| 1052 | IL17RB(55540) |
| 1053 | GTDC1(79712) |
| 1054 | DNAJC18(202052) |
| 1055 | ZNF468(90333) |
| 1056 | ZDHHC11(79844) |
| 1057 | BOLA2(552900) |
| 1058 | BRWD1(54014) |
| 1059 | LRRC15(131578) |
| 1060 | ENAH(55740) |
| 1061 | STK35(140901) |
| 1062 | SUPT7L(9913) |
| 1063 | PTDSS2(81490) |
| 1064 | THSD4(79875) |
| 1065 | IGF1(3479) |
| 1066 | PAX8(7849) |
| 1067 | ENAH(55740) |
| 1068 | ZNF621(285268) |
| 1069 | AFG3L1(172) |
| 1070 | EPB41L4B(54566) |

TABLE 2-30

| | |
|---|---|
| 1071 | PBX1(5087) |
| 1072 | ZNF721(170960) |
| 1073 | LRRC27(80313) |
| 1074 | ABCC3(8714) |
| 1075 | OXCT2(64064) |
| 1076 | CPLX1(10815) |
| 1077 | UBE3B(89910) |
| 1078 | ING5(84289) |
| 1079 | LRPAP1(4043) |
| 1080 | HIF3A(64344) |
| 1081 | PTGDS(5730) |
| 1082 | BOK(666) |
| 1083 | DMWD(1762) |
| 1084 | NFIX(4784) |
| 1085 | ZFP41(286128) |
| 1086 | SNX8(29886) |
| 1087 | AFG3L1(172) |
| 1088 | GIPC3(126326) |
| 1089 | SLC36A4(120103) |
| 1090 | MBNL2(10150) |
| 1091 | WDR37(22884) |
| 1092 | RAPH1(65059) |
| 1093 | SPANX-N1(494118) |
| 1094 | ITIH5(80760) |
| 1095 | PDPK1(5170) |
| 1096 | RAB40C(57799) |
| 1097 | SYT7(9066) |
| 1098 | RAB40C(57799) |
| 1099 | WIPI2(26100) |
| 1100 | ARGFX(503582) |
| 1101 | AP3S2(10239) |
| 1102 | EXOSC6(118460) |
| 1103 | MECP2(4204) |
| 1104 | PDXK(8566) |
| 1105 | TFCP2(7024) |
| 1106 | EIF3S1(8669) |
| 1107 | SNRP70(6625) |
| 1108 | ADCY1(107) |
| 1109 | PDPK1(5170) |
| 1110 | FGD3(89846) |
| 1111 | CBL(867) |
| 1112 | BOK(666) |
| 1113 | ADARB2(105) |
| 1114 | KLK9(284366) |
| 1115 | FN3K(64122) |
| 1116 | NFIX(4784) |

TABLE 2-31

| | |
|---|---|
| 1117 | IL17RE(132014) |
| 1118 | PIGA(5277) |
| 1119 | PRLR(5618) |
| 1120 | EVC(2121) |
| 1121 | SNX8(29886) |
| 1122 | CREB3L2(64764) |
| 1123 | KBTBD11(9920) |
| 1124 | SYNCRIP(10492) |
| 1125 | KIF1B(23095) |
| 1126 | APOL6(80830) |
| 1127 | CBX5(23468) |
| 1128 | RBMS2(5939) |
| 1129 | ZNF721(170960) |
| 1130 | PLXNA1(5361) |
| 1131 | ZNF440L(284390) |
| 1132 | APOE(348) |
| 1133 | NFATC1(4772) |
| 1134 | DISC1(27185) |
| 1135 | MAPK1(5594) |
| 1136 | POLR3H(171568) |
| 1137 | NKTR(4820) |
| 1138 | ABI2(10152) |
| 1139 | APOL6(80830) |
| 1140 | HOXA11(3207) |
| 1141 | RNF12(51132) |
| 1142 | TP53INP1(94241) |
| 1143 | CREB5(9586) |
| 1144 | TPPP(11076) |
| 1145 | H-plk(51351) |
| 1146 | ENTPD7(57089) |
| 1147 | ZNF440L(284390) |
| 1148 | AKNA(80709) |
| 1149 | DFFA(1676) |
| 1150 | FGFRL1(53834) |
| 1151 | RNF12(51132) |
| 1152 | PRX(57716) |
| 1153 | GIPC3(126326) |
| 1154 | RAB30(27314) |
| 1155 | NKTR(4820) |
| 1156 | SEPT5(5413) |
| 1157 | CD59(966) |
| 1158 | CBL(867) |
| 1159 | DDEF2(8853) |
| 1160 | LRPAP1(4043) |
| 1161 | LRPAP1(4043) |
| 1162 | JPH4(84502) |

TABLE 2-32

| | |
|---|---|
| 1163 | TP53INP1(94241) |
| 1164 | ELAVL1(1994) |
| 1165 | ZNF721(170960) |
| 1166 | CMTM4(146223) |
| 1167 | ERBB4(2066) |
| 1168 | GFPT1(2673) |
| 1169 | TOMM20(9804) |
| 1170 | AGPAT3(56894) |
| 1171 | GGTL3(2686) |
| 1172 | ADCY1(107) |
| 1173 | MLX(6945) |
| 1174 | AKNA(80709) |
| 1175 | ADCY1(107) |
| 1176 | OPCML(4978) |
| 1177 | NAV1(89796) |
| 1178 | RIMS3(9783) |
| 1179 | LYNX1(66004) |
| 1180 | LRRC14(9684) |
| 1181 | KCNA7(3743) |
| 1182 | TNRC6B(23112) |
| 1183 | SKIL(6498) |
| 1184 | SBK1(388228) |
| 1185 | ZNF264(9422) |
| 1186 | WDR33(55339) |
| 1187 | EXOSC6(118460) |
| 1188 | APOL6(80830) |
| 1189 | HCP1(113235) |
| 1190 | RASGRP4(115727) |
| 1191 | TFCP2L1(29842) |
| 1192 | CYP4F3(4051) |
| 1193 | BBS5(129880) |
| 1194 | ILF3(3609) |
| 1195 | LYNX1(66004) |
| 1196 | APOE(348) |
| 1197 | TBC1D16(125058) |
| 1198 | RAB40C(57799) |
| 1199 | FUT3(2525) |
| 1200 | PRX(57716) |
| 1201 | PRTG(283659) |
| 1202 | SNX27(81609) |
| 1203 | PIK3CD(5293) |
| 1204 | SLC1A2(6506) |
| 1205 | GGTL3(2686) |
| 1206 | ATP11A(23250) |
| 1207 | ZFP41(286128) |
| 1208 | SOX11(6664) |

TABLE 2-33

| | |
|---|---|
| 1209 | AFG3L1(172) |
| 1210 | FNDC6(152028) |
| 1211 | WNK3(65267) |
| 1212 | ZBTB7A(51341) |
| 1213 | JPH4(84502) |
| 1214 | PRX(57716) |
| 1215 | APOE(348) |
| 1216 | SGCD(6444) |
| 1217 | PRLR(5618) |
| 1218 | STOX2(56977) |
| 1219 | CBX5(23468) |
| 1220 | RNF12(51132) |
| 1221 | EVC(2121) |
| 1222 | DIDO1(11083) |
| 1223 | PRX(57716) |
| 1224 | TBC1D16(125058) |
| 1225 | CBX5(23468) |
| 1226 | SNX8(29886) |
| 1227 | AFG3L1(172) |
| 1228 | MAK10(60560) |
| 1229 | KCNK6(9424) |
| 1230 | EXOC5(10640) |
| 1231 | CDK6(1021) |
| 1232 | QSCN6(5768) |
| 1233 | EVC(2121) |
| 1234 | LRPAP1(4043) |
| 1235 | MDGA1(266727) |
| 1236 | ENTPD7(57089) |
| 1237 | CDR1(1038) |
| 1238 | ADCY1(107) |
| 1239 | CUGBP2(10659) |
| 1240 | ZC3HAV1(56829) |
| 1241 | LRPAP1(4043) |
| 1242 | LASS1(10715) |
| 1243 | FZR1(51343) |
| 1244 | CBX5(23468) |
| 1245 | LRRC14(9684) |
| 1246 | ENTPD7(57089) |
| 1247 | AFG3L1(172) |
| 1248 | DISC1(27185) |
| 1249 | TNRC6B(23112) |
| 1250 | PALM2(114299) |
| 1251 | ZNF721(170960) |
| 1252 | GIPC3(126326) |
| 1253 | CHKB(1120) |
| 1254 | PTPRT(11122) |

TABLE 2-34

| | |
|---|---|
| 1255 | ZNF660(285349) |
| 1256 | ADCY1(107) |
| 1257 | MECP2(4204) |
| 1258 | ZNF264(9422) |
| 1259 | PRX(57716) |
| 1260 | BOK(666) |
| 1261 | RNF12(51132) |
| 1262 | GABRA4(2557) |
| 1263 | WASF2(10163) |
| 1264 | TBC1D16(125058) |
| 1265 | RAB40C(57799) |
| 1266 | SEPT5(5413) |
| 1267 | CHES1(1112) |
| 1268 | TPPP(11076) |
| 1269 | TNRC6B(23112) |
| 1270 | ASB1(51665) |
| 1271 | AP4E1(23431) |
| 1272 | SEC14L1(6397) |
| 1273 | TNNI1(7135) |
| 1274 | POU3F3(5455) |
| 1275 | SEMA5A(9037) |
| 1276 | ABO(28) |
| 1277 | BRWD1(54014) |
| 1278 | MECP2(4204) |
| 1279 | GIPC3(126326) |
| 1280 | THUMPD1(55623) |
| 1281 | GDA(9615) |
| 1282 | FANCC(2176) |
| 1283 | AXL(558) |
| 1284 | POU2AF1(5450) |
| 1285 | PRKAA2(5563) |
| 1286 | RP6-166C19.1(255313) |
| 1287 | REEP3(221035) |
| 1288 | ADAM19(8728) |
| 1289 | TPPP(11076) |
| 1290 | KLF15(28999) |
| 1291 | VGLL4(9686) |
| 1292 | WHSC1(7468) |
| 1293 | TNRC6B(23112) |
| 1294 | TSPY2(64591) |
| 1295 | ZIC1(7545) |
| 1296 | QKI(9444) |
| 1297 | CBX5(23468) |
| 1298 | MAPK1(5594) |
| 1299 | FUT3(2525) |
| 1300 | GNG13(51764) |

TABLE 2-35

| | |
|---|---|
| 1301 | NKTR(4820) |
| 1302 | GOSR1(9527) |
| 1303 | TRIM2(23321) |
| 1304 | BRWD1(54014) |
| 1305 | MECP2(4204) |
| 1306 | NF1(4763) |
| 1307 | GFPT1(2673) |
| 1308 | EVC(2121) |
| 1309 | RP11-114H20.1(401589) |
| 1310 | CPLX2(10814) |
| 1311 | RASSF6(166824) |
| 1312 | NTRK2(4915) |
| 1313 | CALML4(91860) |
| 1314 | RBM33(155435) |
| 1315 | KLK9(284366) |
| 1316 | BRCA1(672) |
| 1317 | FMNL3(91010) |
| 1318 | THRB(7068) |
| 1319 | KLF12(11278) |
| 1320 | CALN1(83698) |
| 1321 | MAGI1(9223) |
| 1322 | TMEM132B(114795) |
| 1323 | FAM78A(286336) |
| 1324 | VGLL3(389136) |
| 1325 | CPLX2(10814) |
| 1326 | NTRK2(4915) |
| 1327 | RIMS4(140730) |
| 1328 | SPTLC2(9517) |
| 1329 | AFG3L1(172) |
| 1330 | RP11-145H9.1(340156) |
| 1331 | NDE1(54820) |
| 1332 | THRB(7068) |
| 1333 | GABRB2(2561) |
| 1334 | RCP9(27297) |
| 1335 | AFG3L1(172) |
| 1336 | PRLR(5618) |

The method of screening a substance that promotes or suppresses the expression or function of a nucleic acid such as a micro-RNA or a micro-RNA precursor using a nucleic acid of the present invention may be any method of screening for a substance that promotes or suppresses the expression or function of a nucleic acid such as a micro-RNA or a micro-RNA precursor using a nucleic acid of the present invention. For example, a method can be mentioned wherein a vector that expresses a nucleic acid, such as a micro-RNA or a micro-RNA precursor, of the present invention, is introduced into a cell, and a substance that suppresses the expression of a target gene thereof, or a substance that promotes the expression of a target gene thereof, is screened for.

Although the substance that suppresses the expression of a target gene may be any substance that suppresses the expression of the mRNA of the target gene, the substance is preferably a nucleic acid, more preferably an siRNA against the target gene. Although the substance that promotes the expression of a target gene may be any substance that promotes the expression of the mRNA of the target gene, the substance is preferably a nucleic acid, more preferably an siRNA against a micro-RNA that suppresses the expression of the target gene.

The cell incorporating a nucleic acid or vector of the present invention may be any cell incorporating the nucleic acid or vector of the present invention introduced in vitro. Specifically, mast cells and mast cell precursor cells, or, mesenchymal stem cells and cells resulting from differentiation of mesenchymal stem cells, for example, cells that are present in tissues such as the skin, lung, small intestine, nose, tonsil, blepharal conjunctiva, vascular walls, and bone marrow, or cells that are present in tissues such as bone marrow, fat tissue, umbilical blood, endometrium, dermis, skeletal muscles, periosteum, dental follicles, periodontal membranes, dental pulps, and dental germs, for example, osteoblasts, adipocytes, and muscle cells and the like, can be mentioned.

In the present invention, a mast cell refers to a cell that becomes activated by various stimuli to undergo degranulation and release or produce many inflammatory mediators, and is involved in the pathogenesis of various allergic diseases.

In the present invention, a mesenchymal stem cell refers to a cell that is present in mesenchymal tissues such as bone marrow, fat tissue, umbilical blood, endometrium, dermis, skeletal muscles, periosteum, dental follicles, periodontal membranes, dental pulps, and dental germs, and has the potential for differentiating at least into mesenchymal cells such as osteoblasts, adipocytes, and muscle cells.

A pharmaceutical with a nucleic acid of the present invention as an active ingredient can be used to diagnose or treat a disease caused by a mast cell abnormality. Because mast cell abnormalities include degranulation abnormalities, a nucleic acid of the present invention can also be used as a mast cell degranulation promoter or degranulation suppressant. A substance that promotes or suppresses the expression or function of a nucleic acid, such as a micro-RNA or a micro-RNA precursor, of the present invention, and a substance that suppresses or promotes the expression of a target gene of a nucleic acid, such as a micro-RNA, of the present invention, can also be used to diagnose or treat a disease caused by a mast cell abnormality. These substances can also be used as mast cell degranulation promoters or degranulation suppressants.

As diseases caused by a mast cell abnormality, specifically, atopic dermatitis, asthma, chronic obstructive lung disease, and allergic diseases and the like can be mentioned.

A pharmaceutical with a nucleic acid of the present invention as an active ingredient can be used to diagnose or treat a disease caused by an abnormality of mesenchymal stem cell proliferation and/or differentiation. A substance that promotes or suppresses the expression or function of a nucleic acid, such as a micro-RNA or a micro-RNA precursor, of the present invention, and a substance that suppresses or promotes the expression of a target gene of a nucleic acid, such as a micro-RNA, of the present invention, can also be used to diagnose or treat a disease caused by an abnormality of mesenchymal stem cell proliferation and/or differentiation.

As diseases caused by an abnormality of mesenchymal stem cell proliferation and/or differentiation, specifically, cancers and dysosteogenesis, achondroplasia, diabetes and the like can be mentioned.

A pharmaceutical with a nucleic acid of the present invention as an active ingredient can be used to diagnose or treat a disease caused by an abnormality of cell proliferation and the like, tissue hyperplasia and the like. A nucleic acid of the present invention can also be used as a cell proliferation suppressant or proliferation promoter. A substance that promotes or suppresses the expression or function of a nucleic acid, such as a micro-RNA or a micro-RNA precursor, of the present invention, and a substance that suppresses or promotes the expression of a target gene of a nucleic acid, such as a micro-RNA, of the present invention, can also be used to diagnose or treat a disease caused by an abnormality of cell proliferation and the like, tissue hyperplasia and the like. These substances can also be used as cell proliferation suppressants or proliferation promoters. Here, an abnormality of cell proliferation refers to a condition wherein cells are proliferating at a rate that is not a normal proliferation rate in a living organism.

As diseases caused by a cell proliferation abnormality, tissue hyperplasia and the like, specifically, cancers, arteriosclerosis, rheumatoid arthritis, prostatic hyperplasia, blood vessel restenosis after percutaneous transvascular coronary angioplasty, fibroid lung, glomerulonephritis, autoimmune diseases and the like can be mentioned.

Hereinafter, the present invention is described in detail with reference to cases wherein the nucleic acid of the present invention is micro-RNA or micro-RNA precursor.

### 1. Identification of Micro-RNAs and Micro-RNA Precursors Expressed in Mast Cells

### (1-1) Acquirement and Cultivation of Mast Cells

The method of acquiring human mast cells is not particularly limited, as far as it ensures safe and efficient acquirement; for example, human mast cells can be prepared from the human lung, skin, fetal liver and the like by known methods [J. Immunol. Methods, 169, 153 (1994); J. Immunol., 138, 861 (1987); J. Allergy Clin. Immunol., 107, 322 (2001); J. Immunol. Methods., 240, 101 (2000)]. Human mast cells can also be prepared by culturing mononuclear cells prepared from human umbilical blood, peripheral blood, bone marrow, lung or skin in the presence of stem cell factor (hereinafter also referred to as SCF) to differentiate them into mast cells in accordance with known methods [J. Immunol., 157, 343, (1996); Blood, 91, 187 (1998); J. Allergy Clin. Immunol., 106, 141 (2000); Blood, 97, 1016 (2001); Blood, 98, 1127 (2001); Blood, 100, 3861 (2002); Blood, 97, 2045 (2001)].

A cell line established from a human mast cell can also be used. As a human mast cell line, LAD2, which is known to well retain the nature of human mast cells [Leuk. Res., 27, 671 (2003); Leuk. Res., 27, 677 (2003)] and the like can be mentioned.

### (1-2) Acquirement of Low-Molecular RNA and Sequence Information

Total RNA is extracted from mast cells acquired by the various methods described above, and using the RNA, a low-molecular RNA comprising a micro-RNA expressed in mast cells can be acquired as described below.

As a method of acquiring a low-molecular RNA, specifically, a method wherein separation and cutting out of a low-molecular RNA by 15% polyacrylamide gel electrophoresis, 5′ terminal dephosphorylation, 3′-adapter ligation, phosphorylation, 5′-adapter ligation, reverse transcription, PCR amplification, concatemerization, and ligation to a vector are performed sequentially, thereafter the low-molecular RNA is cloned, and the nucleotide sequence of the clone is determined, as described in Genes & Development 15, 188-200 (2000), and the like can be mentioned. Alternatively, for example, a method wherein separation and cutting off of a low-molecular RNA by 15% polyacrylamide gel electrophoresis, 5′-adenylation 3′-adapter ligation, 5′-adapter ligation, reverse transcription, PCR amplification, concatemerization, and ligation to a vector are performed sequentially, thereafter the low-molecular RNA is cloned, and the nucleotide sequence of the clone is determined, as described in Science 294, 858-862 (2001), and the like can be mentioned.

Alternatively, separation and cutting off of a low-molecular RNA by 15% polyacrylamide gel electrophoresis, 5′ terminal dephosphorylation, 3′-adapter ligation, phosphorylation, 5′-adapter ligation, reverse transcription, PCR amplification, and ligation to a microbead vector are performed sequentially, thereafter the low-molecular RNA is cloned, and the nucleotide sequence of the microbeads is read to determine the nucleotide sequence, whereby the low-molecular RNA can also be acquired, as described in Nucleic Acids Research 34, 1765-1771 (2006).

A low-molecular RNA can also be acquired using a small RNA Cloning Kit (manufactured by Takara Bio Inc.).

### (1-3) Identification of Micro-RNA

Whether or not the low-molecular RNA sequence acquired is a micro-RNA can be determined on the basis of whether or not the criteria described in RNA, 9, 277-279 (2003) are met. For example, in cases where the low-molecular RNA was acquired and the nucleotide sequence thereof was determined by a method described above, this can be performed as described below.

Specifically, a surrounding genome sequence wherein a DNA sequence corresponding to the nucleotide sequence of the low-molecular RNA acquired is extended by about 50 nt toward the 5′ terminal side and the 3′ terminal side, respectively, is acquired, and the secondary structure of the RNA expected to be transcribed from the genome sequence is predicted. If the result shows that a hairpin structure is present and the nucleotide sequence of the low-molecular RNA is located in one chain of the hairpin, the low-molecular RNA can be judged to be a micro-RNA. Genome sequences are open to the general public, and are available from, for example, UCSC Genome Bioinformatics (http://genome.ucsc.edu/). For prediction of secondary structures, various programs are open; for example, RNAfold [Nucleic Acids Research 31, 3429-3431 (2003)], Mfold [Nucleic Acids Research 31, 3406-3415 (2003)] and the like can be used. Existing micro-RNA sequences are registered in a database called miRBase (http://microrna.sanger.ac.uk/); whether or not a micro-RNA is identical to an existing micro-RNA can be determined on the basis of whether or not the sequence thereof is identical to one of the sequences listed therein.

As examples of the thus-identified micro-RNA expressed in mast cells, a nucleic acid having the nucleotide sequence of any one of SEQ ID NOs:1 to 1336 can be mentioned.

A genome sequence corresponding to the micro-RNA identified and the genome sequence of another organism may be compared, and a nucleic acid having a nucleotide sequence having an identity of 60% or more to the nucleotide sequence of any of SEQ ID NOs:1 to 1336, preferably a nucleic acid having a nucleotide sequence having an identify of 90% or more and more preferably 95% or more, can be identified as a micro-RNA in the organism.

### (1-4) Identification of Micro-RNA Precursor

On the basis of the nucleotide sequence of the micro-RNAs identified in (1-3) above, a sequence comprising the sequence that encodes a micro-RNA can be identified as the sequence that encodes a micro-RNA precursor. As examples of a micro-RNA precursor of the present invention expressed in mast cells, a nucleic acid having the nucleotide sequence of any one of SEQ ID NOs:1337 to 2851 and the like can be mentioned.

Furthermore, a genome sequence corresponding to the micro-RNA precursor identified and the genome sequence of another organism may be compared, and a nucleic acid having a nucleotide sequence having an identity of 80% or more, preferably 90% or more, and more preferably 95% or more, and still more preferably 97% or more, to the nucleotide sequence of any one of SEQ ID NOs:1337 to 2851, can be identified as a micro-RNA precursor in the organism.

### 2. Synthesis of Nucleic Acid

After a micro-RNA and a micro-RNA precursor expressed in mast cells are once identified, as described in 1 above, not only an RNA, which is a polymer of a ribonucleotide, but also a DNA, which is a polymer of a deoxyribonucleotide, can be synthesized on the basis of the nucleotide sequences. For example, on the basis of the nucleotide sequence of the RNA identified in 1 above, the nucleotide sequence of a DNA can be determined. The nucleotide sequence of a DNA corresponding to the nucleotide sequence of an RNA can be determined, without exception, by reading the U (uracil) contained in the sequence of the RNA as T (thymine). A polymer being a mixture of a ribonucleotide and a deoxyribonucleotide and a polymer comprising a nucleotide analogue can also be synthesized in the same manner.

The method of synthesizing a nucleic acid such as micro-RNA or micro-RNA precursor of the present invention is not particularly limited; the same can be produced by a method using a known chemical synthesis, or an enzymatic transcription method and the like. As methods using a known chemical synthesis, the phosphoroamidite method, the phosphorothioate method, the phosphotriester method, the CEM method [Nucleic Acid Research, 35, 3287 (2007)] and the like can be mentioned; for example, a nucleic acid such as micro-RNA or micro-RNA precursor of the present invention can be synthesized using the ABI3900 high throughput nucleic acid synthesizer (manufactured by Applied Biosystems). As an enzymatic transcription method, transcription with a plasmid or DNA having a desired nucleotide sequence as the template using a typical phage RNA polymerase, for example, T7, T3, or SP6RNA polymerase, can be mentioned.

### 3. Method of Detecting Functions of Micro-RNA and Precursor of the Micro-RNA

A micro-RNA is produced via processing of a micro-RNA precursor having a hairpin structure by a protein called Dicer, a kind of RNaseIII endonuclease, in cytoplasm, and suppresses the translation of an mRNA having a target nucleotide sequence. Therefore, whether or not the nucleic acid obtained is a micro-RNA can be determined on the basis of whether or not the function is present.

For example, on the basis of whether or not the RNA undergoes processing by RNaseIII endonuclease, whether or not the same functions as a micro-RNA precursor can be measured. Specifically, a single-stranded RNA whose function is to be detected is reacted with RNaseIII endonuclease, and the reaction product is electrophoresed; if a function as a micro-RNA precursor is possessed, a band about 20 to 25 nucleotides long resulting from the processing will be detected, whereby the RNA is judged to possess a function as a micro-RNA. Although an RNaseIII endonuclease is not particularly limited, as far as it possesses an activity to process the micro-RNA precursor, it is preferable to use a Dicer protein. Specifically, si-RNAse III™ (manufactured by Takara Bio Inc.), Cold Shock-DICER (manufactured by Takara Bio Inc.), Recombinant Dicer Enzyme (manufactured by Stratagene), BLOCK-iT Dicer RNAi Transfection Kit (manufactured by Invitrogen), X-treme GENE siRNA Dicer Kit (manufactured by Roche-Applied Science) and the like can be used, and the measurement can be made according to the reaction conditions given in the attached instructions.

As a method of detecting a function of a micro-RNA, a method can be mentioned wherein the function is measured on the basis of whether or not the translation of a mRNA having a target nucleotide sequence is suppressed.

Micro-RNAs are known to suppress the translation of an mRNA comprising a target nucleotide sequence thereof in the untranslated region on the 3′ side (3′UTR) [Current Biology, 15, R458-R460 (2005)]. Hence, a DNA wherein a target nucleotide sequence for the single-stranded RNA to be measured is inserted into the 3′UTR of an appropriate reporter gene expression vector is prepared and introduced into a host cell suitable for the expression vector, and the expression of the reporter gene is measured when the cell is allowed to express the single-stranded. RNA, whereby whether or not a function of a micro-RNA is possessed can be detected.

The reporter gene expression vector may be any one, as far as it has a promoter upstream of a reporter gene, and is capable of expressing the reporter gene in the host cell. Any reporter gene can be used; for example, the firefly luciferase gene, the Renilla luciferase gene, the chloramphenicol acetyltransferase gene, the β-glucuronidase gene, the β-galactosidase gene, the β-lactamase gene, the aequorin gene, the green fluorescent protein gene, the DsRed fluorescent gene and the like can be utilized. As examples of reporter gene expression vectors having these properties, psiCHECK-1 (manufactured by Promega), psiCHECK-2 (manufactured by Promega), pGL3-Control (manufactured by Promega), pGL4 (manufactured by Promega), pRNAi-GL (manufactured by Takara Bio Inc.), pCMV-DsRed-Express (manufactured by CLONTECH) and the like can be mentioned. An RNA can be expressed by the method described in 6 below.

A function of a micro-RNA can be detected, specifically as described below. First, a host cell is cultured on a multiwell plate or the like, and a reporter gene expression vector having a target nucleotide sequence and an RNA are expressed. Thereafter, reporter activity is measured, and a reduction in the reporter activity is detected when the RNA is expressed compared with the RNA being not expressed, whereby a function of the micro-RNA can be detected.

### 4. Method of Detecting the Expression of a Nucleic Acid Such as a Micro-RNA or a Micro-RNA Precursor Using a Nucleic Acid of the Present Invention

Hereinafter, methods of detecting the expression of a nucleic acid such as a micro-RNA or a precursor thereof using a nucleic acid of the present invention are described.

As examples of methods of detecting the expression of a 15 micro-RNA, a micro-RNA precursor and the like, (1) Northern hybridization, (2) dot blot hybridization, (3) in situ hybridization, (4) quantitative PCR, (5) differential hybridization, (6) microarray, (7) ribonuclease protection assay and the like can be mentioned.

The Northern blot method is a method wherein a sample-derived RNA is separated by gel electrophoresis, then transferred to a support such as a nylon filter, and an appropriately-labeled probe is prepared on the basis of the nucleotide sequence of a nucleic acid of the present invention, and hybridization and washing are performed, whereby a band specifically bound to the nucleic acid of the present invention is detected; specifically, for example, this method can be performed as described in Science 294, 853-858 (2001) and the like.

A labeled probe can be prepared by incorporating a radioisotope, biotin, digoxigenin, a fluorescent group, a chemiluminescent group and the like in a DNA, RNA, or LNA and the like having a sequence complementary to the nucleotide sequence of a nucleic acid of the present invention by a method, for example, nick translation, random priming or 5′-terminal phosphorylation. Because the amount of labeled probe bound reflects the expression level of a nucleic acid such as a micro-RNA or a micro-RNA precursor, the expression level of a micro-RNA, micro-RNA precursor or the like can be quantified by quantifying the amount of labeled probe bound. Electrophoresis, membrane transfer, probe preparation, hybridization, and nucleic acid detection can be achieved by a method described in Molecular Cloning, 3rd edition, and Cold Spring Harbor Laboratory Press, NY, USA (2001).

Dot blot hybridization is a method wherein a nucleic acid extracted from a tissue or a cell is spotted in dot forms and immobilized on a membrane, and hybridized with a probe, and a nucleic acid that specifically hybridizes with the probe is detected. The probe used may be the same as that used for Northern hybridization. A nucleic acid preparation, spotting, hybridization, and detection can be achieved by a method described in Molecular Cloning, 3rd edition.

In situ hybridization is a method wherein a paraffin-embedded or cryostat-treated section of a tissue acquired from a living organism, or a cell fixed, is used as a sample and subjected to steps for hybridization with a labeled probe and washing, and the distribution and localization of a micro-RNA, micro-RNA precursor and the like in the tissue or cell are examined by microscopic examination [Methods in Enzymology, 254, 419 (1995)]. The probe used may be the same as that used for Northern hybridization. Specifically, a micro-RNA, micro-RNA precursor and the like can be detected in accordance with a method described in Nature Method 3, 27 (2006).

In quantitative PCR, a cDNA synthesized from a sample-derived RNA using a primer for reverse transcription and a reverse transcriptase (hereunder, this cDNA is referred to as a sample-derived cDNA) is used for the measurement. As a primer for reverse transcription to be supplied for cDNA synthesis, a random primer or a specific RT primer and the like can be used. A specific RT primer refers to a primer having a sequence complementary to a nucleotide sequence corresponding to a micro-RNA, micro-RNA precursor of the present invention and the like, and a genome sequence therearound.

For example, a sample-derived cDNA is synthesized, after which a PCR is performed with this cDNA as the template, using a template-specific primer designed from a nucleotide sequence corresponding to a micro-RNA, micro-RNA precursor and a genome sequence therearound, or from a nucleotide sequence corresponding to a primer for reverse transcription, to amplify a cDNA fragment and the amount of the micro-RNA and micro-RNA precursor contained in the sample-derived RNA is detected from the number of cycles for reach to a given amount of the fragment. As the template-specific primer, an appropriate region corresponding to a micro-RNA, micro-RNA precursor and a genome sequence therearound is selected, and a pair of a DNA or LNA consisting of a sequence of 20 to 40 nucleotides at the 5′ terminus of the nucleotide sequence of the region, and a DNA or LNA consisting of a sequence complementary to a sequence of 20 to 40 nucleotides at the 3′ terminus can be used. Specifically, this can be performed in accordance with a method described in Nucleic Acids Research, 32, e43 (2004) and the like.

Alternatively, as the primer for reverse transcription to be supplied for cDNA synthesis, a specific RT primer having a stem-loop structure can also be used. Specifically, this can be performed using a method described in Nucleic Acid Research, 33, e179 (2005), or TaqMan MicroRNA Assays (manufactured by Applied Biosystems).

As another method of synthesizing a sample-derived cDNA, a polyA sequence is added to a sample-derived RNA by means of polyA polymerase, and a nucleotide sequence comprising an oligodT sequence is used as a primer for reverse transcription, whereby a reverse transcription reaction can be performed. Specifically, this can be performed using the miScript System (manufactured by QIAGEN) or the QuantiMir RT Kit (manufactured by System Biosciences).

In addition, by hybridizing a sample-derived cDNA to a substrate such as a filter, glass slide, or silicone having a DNA or LNA corresponding to a nucleotide sequence comprising at least one or more of a nucleic acid such as micro-RNA, micro-RNA precursor of the present invention and the like immobilized thereon, and performing washing, a change in the amount of the micro-RNA, micro-RNA precursor of the present invention and the like can be detected. As such methods based on hybridization, methods using differential hybridization [Trends Genet., 7, 314 (1991)] or a microarray [Genome Res., 6, 639 (1996)] can be mentioned. Both methods enable accurate detection of a difference in the amount of a micro-RNA, a micro-RNA precursor or the like between a control sample and a target sample by immobilizing an internal control, such as a nucleotide sequence corresponding to U6 RNA, on a filter or a substrate. Also, by synthesizing labeled cDNAs using differently labeled dNTPs (mixtures of dATP, dGTP, dCTP, and dTTP) on the basis of RNAs derived from a control sample and a target sample, and simultaneously hybridizing the two labeled cDNAs to a single filter or a single substrate, accurate quantitation of the micro-RNA, micro-RNA precursor and the like can be performed. Furthermore, quantitation of a micro-RNA, a micro-RNA precursor or the like can also be performed by directly labeling and hybridizing an RNA derived from a control sample and/or a target sample. For example, a micro-RNA or the like can be detected using microarrays described in Proc. Natl. Acad. Sci. USA, 101, 9740-9744 (2004), Nucleic Acid Research, 32, e188 (2004) and the like. Specifically, a micro-RNA or the like can be detected or quantified using mirVana miRNA Bioarray (manufactured by Ambion).

In ribonuclease protection assay, first, a promoter sequence such as the T7 promoter or the SP6 promoter is bound to the 3′ terminus of a nucleotide sequence corresponding to a micro-RNA, micro-RNA precursor of the present invention or a genome sequence therearound, and a labeled antisense RNA is synthesized with an in vitro transcription system using a labeled NTP (a mixture of ATP, GTP, CTP, and UTP) and an RNA polymerase. The labeled antisense RNA is bound to a sample-derived RNA to form an RNA-RNA hybrid, after which the hybrid is digested with ribonuclease A, which degrades single-stranded RNAs only. The digest is subjected to gel electrophoresis to detect or quantify an RNA fragment protected against the digestion by forming the RNA-RNA hybrid, as a micro-RNA or micro-RNA precursor. Specifically, the fragment can be detected or quantified a micro-RNA and the like using the mirVana miRNA Detection Kit (manufactured by Ambion).

### 5. Method of Detecting Mutations of a Nucleic Acid Such as Micro-RNA, Micro-RNA Precursor and the Like Using a Nucleic Acid of the Present Invention

Hereinafter, methods of detecting mutations of a nucleic acid such as a micro-RNA or a micro-RNA precursor using a nucleic acid of the present invention are described.

As a method of detecting mutations of a micro-RNA, micro-RNA precursor and the like, a method can be used wherein a heteroduplex formed by hybridization of the normal form of the micro-RNA and a mutated form of the micro-RNA, or of the normal form of the micro-RNA precursor and a mutated form of the micro-RNA precursor, is detected.

As methods of detecting a heteroduplex, (1) detection of a heteroduplex by polyacrylamide gel electrophoresis [Trends genet., 7, 5 (1991)], (2) single-strand conformation polymorphism analysis [Genomics, 16, 325-332 (1993)], (3) chemical cleavage of mismatches (CCM) [Human Genetics (1996), Tom Strachan and Andrew P. Read, BIOS Scientific Publishers Limited], (4) enzymatic cleavage of mismatches [Nature Genetics, 9, 103-104 (1996)], (5) denatured gel electrophoresis [Mutat. Res., 288, 103-112 (1993)] and the like can be mentioned.

Detection of a heteroduplex by polyacrylamide gel electrophoresis is, for example, performed as described below. First, with a sample-derived DNA or a sample-derived cDNA as the template, and using a primer designed on the basis of a genome nucleotide sequence comprising the nucleotide sequence of a micro-RNA and micro-RNA precursor of the present invention and the like, a fragment smaller than 200 by is amplified. Heteroduplexs, if formed, are slower in mobility than mutation-free homo-double-strands, and can be detected as extra bands. Better separation is achieved using a custom-made gel (Hydro-link, MDE and the like). In the case of search for a fragment smaller than 200 bp, insertions, deletions, and most single-nucleotide substitutions can be detected. It is desirable that heteroduplex analysis be performed using a single gel in combination with the single strand conformation analysis described below.

In single strand conformation polymorphism analysis (SSCP analysis), a DNA amplified as a fragment smaller than 200 by with a sample-derived DNA or sample-derived cDNA as the template, using a primer designed on the basis of a genome nucleotide sequence comprising the nucleotide sequence of a micro-RNA, micro-RNA precursor and the like, is denatured, after which it is electrophoresed in non-denatured polyacrylamide gel. By labeling the primer with an isotope or a fluorescent dye at the time of DNA amplification, or by silver-staining the non-labeled amplification product, the amplified DNA can be detected as a band. To clarify a difference from the wild type pattern, a control sample may be electrophoresed simultaneously, whereby a fragment with a mutation can be detected on the basis of a difference in mobility.

In chemical cleavage of mismatches (CCM method), a DNA fragment amplified with a sample-derived DNA or sample-derived cDNA as the template, using a primer designed on the basis of a genome nucleotide sequence comprising the nucleotide sequence of a micro-RNA, micro-RNA precursor and the like, is hybridized to a labeled nucleic acid prepared by allowing a nucleic acid of the present invention to incorporate an isotope or a fluorescent target, and treated with osmium tetraoxide to cleave one strand of the DNA at the mismatched portion, whereby a mutation can be detected. CCM is one of the most sensitive methods of detection, and can be applied to samples of kilobase length.

In place of osmium tetraoxide used above, T4 phage resolvase and an enzyme involved in mismatch repair in cells, such as endonuclease VII, and RNaseA may be used in combination to enzymatically cleave a mismatch.

In denaturing gradient gel electrophoresis (DGGE method), a DNA amplified with a sample-derived DNA or sample-derived cDNA as the template, using a primer designed on the basis of a genome nucleotide sequence comprising the nucleotide sequence of a micro-RNA, micro-RNA precursor and the like, is electrophoresed using a gel having a chemical denaturant density gradient or a temperature gradient. The DNA fragment amplified migrates in the gel to a position where it denatures to a single strand, and no longer migrates after the denaturation. Because the migration of the amplified DNA in the gel differs between the presence and absence of a mutation, the presence of the mutation can be detected. To increase the detection sensitivity, the addition of a poly (G:C) end to each primer is effective.

By directly determining and analyzing the nucleotide sequence of a sample-derived DNA or sample-derived cDNA, a mutation of a micro-RNA, micro-RNA precursor and the like can also be detected.

### 6. Method of Expressing a Nucleic Acid Such as Micro-RNA or Micro-RNA Precursor of the Present Invention, and the Like

A nucleic acid of the present invention, such as a micro-RNA or a micro-RNA precursor, can be expressed by using an expression vector that encodes the nucleic acid.

As an expression vector, a plasmid, viral vector or the like capable of self-replication in the host cell, or capable of being incorporated in the chromosome, that comprises a promoter at a position enabling the transcription of the gene of a nucleic acid, such as a micro-RNA or a micro-RNA precursor, of the present invention of the present invention is preferably used. The promoter may be any one, as far as it is capable of expressing in the host cell; for example, a RNA polymerase II (pol II) system promoter, a RNA polymerase III (pol III) system promoter being a U6 RNA and H1 RNA transcription system and the like can be mentioned. As examples of pol II system promoters, the promoter of the cytomegalovirus (human CMV) IE (immediate early) gene, the early promoter of SV40 and the like can be mentioned. As examples of expression vectors using them, pCDNA6.2-GW/miR (manufactured by Invitrogen), pSilencer 4.1-CMV (manufactured by Ambion) and the like can be mentioned. As pol III system promoters, U6 RNA, H1 RNA or tRNA promoters can be mentioned. As examples of expression vectors using them, pSINsi-hH1 DNA (manufactured by Takara Bio Inc.), pSINsi-hU6 DNA (manufactured by Takara Bio Inc.), pENTR/U6 (manufactured by Invitrogen) and the like can be mentioned. When a micro-RNA, a micro-RNA precursor or the like is to be expressed in vitro, an expression vector having the T7 promoter, the T3 promoter or the SP6 promoter is preferably used. As examples of vectors having these promoters, pBluescript II SK(+) (manufactured by Stratagene) and the like can be mentioned.

When a plasmid is used, by preparing a DNA fragment comprising a hairpin portion on the basis of the nucleotide sequence of a micro-RNA, a micro-RNA precursor or the like of the present invention, or the nucleotide sequence of a genome comprising the foregoing nucleotide sequence, and inserting the fragment downstream of a promoter of the plasmid to construct a recombinant plasmid, and then introducing the plasmid into a host cell suitable for the plasmid, or mixing the plasmid with RNA polymerase, a nucleotide or the like in vitro, a nucleic acid, such as a micro-RNA or a micro-RNA precursor, of the present invention, can be expressed.

When a viral vector is used, by inserting a gene comprising the nucleotide sequence of a nucleic acid, such as a micro-RNA or a micro-RNA precursor, of the present invention, downstream of a promoter in the viral vector to construct a recombinant viral vector, and introducing the vector into a packaging cell to produce a recombinant virus, the gene of the nucleic acid such as the micro-RNA or micro-RNA precursor can be expressed.

The packaging cell may be any cell, as far as it is capable of supplementing a recombinant viral vector deficient in any one of the genes that encode the proteins necessary for the packaging of the virus with the lacked protein; for example, human kidney-derived HEK293 cells, mouse fibroblasts NIH3T3-derived cells and the like can be used. As the protein supplemented by the packaging cell, in the case of a retrovirus vector, proteins derived from a mouse retrovirus, such as gag, pol, and env, can be used; in the case of a lentivirus vector, proteins derived from a HIV virus, such as gag, pol, env, vpr, vpu, vif, tat, rev, and nef, can be used; in the case of an adenovirus vector, proteins derived from an adenovirus, such as E1A and E1B, can be used; in the case of an adeno-associated viral vector, proteins such as Rep (p5, p19, p40) and Vp(Cap), can be used.

In addition to using an expression vector, a nucleic acid such as micro-RNA and micro-RNA precursor of the present invention and the like can also be introduced directly into a cell, without using a vector. As the nucleic acid used in this technique, a DNA, an RNA, or a nucleotide analogue, as well as a chimeric molecule thereof, or a derivative of the nucleic acid can also be used. Specifically, Pre-miR™ miRNA Precursor Molecules (manufactured by Ambion) and miRIDIAN microRNA Mimics (manufactured by GE Healthcare) can be used.

### 7. Methods of Promoting or Suppressing or Promoting the Expression or Function of a Nucleic Acid Such as a Micro-RNA or Micro-RNA Precursor of the Present Invention

The expression or function of a nucleic acid such as micro-RNA and micro-RNA precursor of the present invention and the like can be suppressed using an antisense technology [Baiosaiensu To Indasutorii, 50, 322 (1992); Kagaku, 46, 681 (1991), Biotechnology, 9, 358 (1992), Trends in Biotechnology, 10, 87 (1992), Trends in Biotechnology, 10, 152 (1992); SAIBO KOGAKU, 16, 1463 (1997)], triple helix technology [Trends in Biotechnology, 10, 132 (1992)], ribozyme technology [Current Opinion in Chemical Biology, 3, 274 (1999), FEMS Microbiology Reviews, 23, 257 (1999), Frontiers in Bioscience, 4, D497 (1999), Chemistry & Biology, 6, R33 (1999), Nucleic Acids Research, 26, 5237 (1998), Trends In Biotechnology, 16, 438 (1998)], decoy DNA method [Nippon Rinsho—Japanese Journal of Clinical Medicine, 56, 563 (1998), Circulation Research, 82, 1023 (1998), Experimental Nephrology, 5, 429 (1997), Nippon Rinsho—Japanese Journal of Clinical Medicine, 54, 2583 (1996)], or a siRNA (short interfering RNA).

An antisense refers to one that allows nucleotide sequence-specific hybridization of a nucleic acid having a nucleotide sequence complementary to a certain target nucleic acid to suppress the expression or function of the target nucleic acid. As the nucleic acid used as the antisense, a DNA, an RNA or a nucleotide analogue, as well as a chimeric molecule thereof, or a derivative of the nucleic acid can also be used. Specifically, an antisense can be prepared by following the method described in Nature 432, 226 (2004) and the like, and the expression or function can be suppressed. Specifically, by using Anti-miR™ miRNA Inhibitors (manufactured by Ambion) or miRIDIAN microRNA Inhibitors (manufactured by GE Healthcare), the expression or function of a nucleic acid, such as a micro-RNA or a micro-RNA precursor, of the present invention, can be suppressed.

An siRNA refers to a short double-stranded RNA comprising the nucleotide sequence of a certain target nucleic acid, that is capable of suppressing the expression or function of the target nucleic acid by RNA interference (RNAi). The sequence of an siRNA can be designed as appropriate from the target nucleotide sequence on the basis of conditions shown in the literature [Genes Dev., 13, 3191 (1999)]. By synthesizing two RNAs having a sequence of 17 to 30 nucleotides, preferably 18 to 25 nucleotides, more preferably 19 to 23 nucleotides, selected and a complementary sequence, with TT added to the 3′ terminus of each thereof, using a nucleic acid synthesizer, and performing annealing, an siRNA can be prepared. By inserting a DNA corresponding to the above-described selected sequence of 17 to 30 nucleotides, preferably 18 to 25 nucleotides, more preferably 19 to 23 nucleotides, into an siRNA expression vector such as pSilencer 1.0-U6 (manufactured by Ambion) or pSUPER (OligoEngine), a vector that expresses an siRNA capable of suppressing the expression or function of the gene can also be prepared.

Using an antisense or siRNA specific for a micro-RNA or micro-RNA precursor expressed in mast cells, mesenchymal stem cells or cancer cells, the expression or function of a micro-RNA or micro-RNA precursor expressed in mast cells, mesenchymal stem cells or cancer cells can be suppressed. Specifically, by administering the antisense or siRNA specific for a micro-RNA, the expression or function of the micro-RNA can be suppressed, and the action of the micro-RNA or micro-RNA precursor in mast cells, mesenchymal stem cells or cancer cells can be controlled.

Referring to a specific example, an antisense or siRNA specific for a micro-RNA of any one of SEQ ID NOs:1, 2, 3, 8, 14, 20, 22, 25, 32 and 36 or a micro-RNA precursor of any one of SEQ ID NOs:1337, 1338, 1339, 1352, 1363, 1371, 1373, 1377, 1386 and 1390 can be used as a mast cell degranulation suppressant. An antisense or siRNA specific for a micro-RNA of SEQ ID NOs:1, 8, 21 and 36 or a micro-RNA precursor of any one of SEQ ID NOs:1337, 1352, 1372 and 1390 can be used as a mesenchymal stem cell proliferation suppressant or proliferation promoter. Furthermore, an antisense or siRNA specific for a micro-RNA of any one of SEQ ID NOs:1, 3, 8, 20, 21, 22, 32 and 36 or a micro-RNA precursor of any one of SEQ ID NOs:1337, 1339, 1352, 1371, 1372, 1373, 1386 and 1390 can be used as a cell proliferation promoter.

In the case of a patient affected by an abnormality of the expression of a micro-RNA or micro-RNA precursor expressed in mast cells, mesenchymal stem cells or cancer cells, by administering an antisense or siRNA specific for the micro-RNA or precursor thereof to the patient, it is possible to control a function of mast cells, mesenchymal stem cells or cancer cells to treat a disease that develops as a result of the above-described expressional abnormality. Hence, an antisense or siRNA that is specific for the micro-RNA or precursor thereof is useful as a therapeutic agent for a disease caused by an abnormality of mast cells or mesenchymal stem cells, or a disease caused by a cell proliferation abnormality.

When an antisense or siRNA that is specific for the micro-RNA or precursor thereof is used as the above-described therapeutic agent, the antisense or siRNA, alone or after being inserted into an appropriate vector such as a retrovirus vector, adenovirus vector, or adeno-associated viral vector, can be administered in the form of a pharmaceutical preparation prepared by the conventional method described in 11 below.

Meanwhile, as substances that promote the expression or function of a nucleic acid, such as a micro-RNA or a micro-RNA precursor, of the present invention, the micro-RNA and the micro-RNA precursor, a micro-RNA and micro-RNA precursor that share the same target nucleotide sequence therewith, and an expression vector that encodes the same, as well as factors involved in the processing of micro-RNA precursors, such as Dicer, can be mentioned. An expression vector that encodes a micro-RNA, micro-RNA precursor or the like can be produced by the methods mentioned in the foregoing 6.

Using a micro-RNA or micro-RNA precursor expressed in mast cells, mesenchymal stem cells or cancer cells, a micro-RNA or micro-RNA precursor that shares the same target nucleotide sequence therewith, and an expression vector that encodes the same, the expression or function of a micro-RNA or micro-RNA precursor expressed in mast cells, mesenchymal stem cells or cancer cells can be promoted. Hence, by administering them, it is possible to promote the expression or function of the micro-RNA to control the action of micro-RNA and micro-RNA precursor in mast cells, mesenchymal stem cells or cancer cells.

Referring to specific examples, the micro-RNA of any one to of SEQ ID NOs:1, 2, 3, 8, 14, 20, 22, 25, 32 and 36 or the micro-RNA precursor of any one of SEQ ID NOs:1337, 1338, 1339, 1352, 1363, 1371, 1373, 1377, 1386 and 1390, a micro-RNA or micro-RNA precursor that shares the same target nucleotide sequence therewith, and an expression vector that encodes the same can be used as mast cell degranulation promoters. The micro-RNA of any one of SEQ ID NOs:1, 8, 21 and 36 or the micro-RNA precursor of any one of SEQ ID NOs:1337, 1352, 1372 and 1390, a micro-RNA or micro-RNA precursor that shares the same target nucleotide sequence therewith, and an expression vector that encodes the same can be used as proliferation promoters or proliferation suppressants of mesenchymal stem cells. Furthermore, the micro-RNA of any one of SEQ ID NOs:1, 3, 8, 20, 21, 22, 32 and 36 or the micro-RNA precursor of any one of SEQ ID NOs:1337, 1339, 1352, 1371, 1372, 1373, 1386 and 1390, a micro-RNA or micro-RNA precursor that shares the same target nucleotide sequence therewith, and an expression vector that encodes the same can be used as cell proliferation suppressants.

In the case of a patient affected by an abnormality of the expression of a micro-RNA or micro-RNA precursor expressed in mast cells, mesenchymal stem cells or cancer cells, by administering the micro-RNA, precursor thereof, an expression vector that encodes the same, a micro-RNA or micro-RNA precursor that shares the same target nucleotide sequence therewith, and an expression vector that encodes the same to the patient, it is possible to control a function of mast cells, mesenchymal stem cells or cancer cells to treat the above-described disease that develops as a result of an expressional abnormality. Hence, the micro-RNA or precursor thereof, an expression vector that encodes the same, a micro-RNA or micro-RNA precursor that shares the same target nucleotide sequence therewith, and an expression vector that encodes the same are useful as therapeutic agents for a disease caused by an abnormality of mast cells or mesenchymal stem cells, or a disease caused by a cell proliferation abnormality.

When the micro-RNA or a precursor thereof, and an expression vector that encodes the same are used as the above-described therapeutic agent, the micro-RNA or precursor thereof, and the expression vector that encodes the same, alone or using an appropriate vector such as a retrovirus vector, adenovirus vector, or adeno-associated viral vector, can be administered in the form of a pharmaceutical preparation prepared by the conventional method described in 11 below.

### 8. Methods of Suppressing or Promoting the Expression of a Target Gene of a Nucleic Acid, Such as a Micro-RNA, of the Present Invention

The method of suppressing the expression of a target gene of a nucleic acid, such as a micro-RNA, of the present invention, may be any method, as far as the expression of the target gene is suppressed. For example, a method can be mentioned wherein a nucleic acid, such as a micro-RNA or a micro-RNA precursor, of the present invention, is expressed or administered to increase the amount of micro-RNA in the cell, whereby the expression of an mRNA having the target sequence is suppressed to suppress the expression of the target gene. Here, a nucleic acid, such as a micro-RNA or a micro-RNA precursor, of the present invention, can be expressed by the methods described in 6 above.

The method of promoting the expression of a target gene of a nucleic acid, such as a micro-RNA, of the present invention, may be any method, as far as the expression of the target gene is promoted. For example, a method can be mentioned wherein an antisense or siRNA against a nucleic acid, such as a micro-RNA or a micro-RNA precursor, of the present invention, is expressed or administered to suppress the expression or function of the micro-RNA, micro-RNA precursor and the like, whereby the expression of the target gene is promoted. The antisense and siRNA can be prepared by the methods described in 7 above.

As examples of a target gene of a micro-RNA consisting of the nucleotide sequence of any one of SEQ ID NOs:1 to 1336, the above-described gene cluster shown in Table 2 can be mentioned.

### 9. Method of Separating Cells Using a Nucleic Acid Such as Micro-RNA and Micro-RNA Precursor of the Present Invention, and the Like

As a method of separating cells that express a nucleic acid such as the micro-RNA and micro-RNA precursor of the present invention, and the like from various cells taken out from a living organism, a probe prepared by fluorescently labeling a nucleic acid having a nucleotide sequence complementary to the nucleotide sequence of a nucleic acid such as micro-RNA, micro-RNA precursor and the like is introduced to a cell to hybridize the same to the nucleic acid such as micro-RNA, micro-RNA precursor and the like, and only the cells that have hybridized with the labeled probe are separated using a flow cytometer with a sorting function.

The fluorescently labeled probe may be any one, as far as it emits specific fluorescence upon hybridization; for example, a molecular beacon [Biochimica et Biophysica Acta 1479, 178 (1998)), FRET [Proc. Natl. Acad. Sci. USA 103, 263 (2006)] and the like can be mentioned.

A molecular beacon is a nucleic acid wherein a fluorescent functional group has been introduced via a covalent bond at one end thereof, and a dabsyl group and the like that cause fluorescence quenching has been introduced at the other end, and the nucleotide sequence thereof is designed to assume a hairpin structure in an ordinary aqueous solution. Because the fluorescent functional group and the fluorescence quenching group introduced to both ends are adjacent to each other, no fluorescence is observed with the probe alone, but if the probe is hybridized with a nucleic acid such as micro-RNA and micro-RNA precursor of the present invention, and the like, the fluorescent functional group and the fluorescence quenching group are disjoined, and intense fluorescence is observable.

FRET is the phenomenon in which molecule excitation energy transfer occurs between two kinds or more of fluorescent functional groups. Specifically, two nucleic acid probes are provided: a nucleic acid probe incorporating an energy donor at one end thereof and another nucleic acid probe incorporating an energy receptor at the other end. If the nucleotide sequences of the two probes are designed to allow the two fluorescent functional groups, introduced after hybridization with a nucleic acid such as micro-RNA, micro-RNA precursor and the like, to come into close contact with each other, only emission from the donor is observed with the probe alone, but if the probes are hybridized with a nucleic acid such as the micro-RNA and micro-RNA precursor of the present invention, and the like, the two probes come in close contact with each other, the energy of the donor transfers to the acceptor, and emission based on the acceptor is mainly observable.

As methods of cell separation using a flow cytometer with a sorting function, the water charge method, the cell capture method and the like can be mentioned (Huro Saitometa Jiyu Jizai, p 14-23, SHUJUNSHA, 1999). In both methods, cell fluorometry is performed and fluorescence intensity is converted to electrical signals to quantify fluorescence intensity, whereby cells can be separated according to the amount quantified. Specifically, fluorescence intensity can be measured using the BD FACS Aria cell sorter (manufactured by Becton Dickinson Immunocytometry Systems), EPICS ALTRA HyPerSort (manufactured by Beckman Coulter, K.K.) and the like, and the cells can be separated.

### 10. Method of Screening for a Substance That Promotes or Suppresses the Expression or Function of a Nucleic Acid Such as a Micro-RNA or Micro-RNA Precursor of the Present Invention

Using a nucleic acid, such as a micro-RNA or a micro-RNA precursor, of the present invention, a substance that promotes or suppresses the expression or function of the micro-RNA or precursor thereof can be screened for. For example, from among the nucleotide sequences of micro-RNAs and micro-RNA precursors of the present invention, a nucleotide sequence to be targeted for the screening is chosen, and by means of a cell that expresses a nucleic acid having the nucleotide sequence, a substance that promotes or suppresses the expression or function of the chosen micro-RNA or precursor thereof can be screened for.

As cells that express a nucleic acid having the nucleotide sequence of a micro-RNA or micro-RNA precursor, used for screening, mast cells, mesenchymal stem cells or cancer cells, as well as transformant cells obtained by introducing a vector that expresses a nucleic acid having the nucleotide sequence into a host cell such as an animal cell or yeast, cells incorporating a nucleic acid having the nucleotide sequence introduced directly without using a vector and the like as described in 6 above can also be used.

As specific methods of screening, (a) a method wherein a change in the expression level of a micro-RNA or precursor thereof being a target for screening is used as an index, as well as (b) a method wherein a change in the expression level of an mRNA having a target sequence of a micro-RNA or precursor thereof being a target for screening is used as an index, since a micro-RNA suppresses the translation of an mRNA having a target sequence, can be mentioned.

### (a) Screening Method Wherein a Change in the Expression Level of a Micro-RNA or Precursor Thereof Being a Target for Screening is Used as an Index

A test substance is brought into contact with a cell that expresses a nucleic acid having the nucleotide sequence, and with a change in the expression level of the nucleic acid selected as an index, a substance that promotes or suppresses the expression of a micro-RNA and precursor thereof is obtained. The expression level of a nucleic acid can be detected by the method described in 4 above.

(b) Screening Method Wherein a Change in the Expression Level of an mRNA Having a Target Sequence of a Micro-RNA or Precursor Thereof Being a Target for Screening is Used as an Index

A test substance is brought into contact with a cell that expresses a nucleic acid having the nucleotide sequence, and with a change in the expression level of an mRNA having a target sequence of the nucleic acid selected as an index, a substance that promotes or suppresses the expression or function of a micro-RNA and a precursor thereof is obtained. Alternatively, a DNA incorporating a target sequence for a single-stranded RNA having a nucleotide sequence of the present invention introduced into the 3′ UTR of an appropriate reporter gene expression vector is prepared and introduced into a host cell suitable for the expression vector, a test substance is brought into contact with the cell, and with a change in the expression level of the reporter gene as an index, a substance that promotes or suppresses the expression or function of a micro-RNA and precursor thereof is obtained.

A target sequence can be selected by the method described above; as examples of a target gene of a micro-RNA consisting of the nucleotide sequence of any one of SEQ ID NOs:1 to 1336, the above-described gene cluster shown in Table 2 can be mentioned.

### 11. Diagnostic Reagents and Therapeutic Agents Comprising a Nucleic Acid of the Present Invention and the Like

A nucleic acid, such as a micro-RNA or a micro-RNA precursor, of the present invention, and a nucleic acid having a nucleotide sequence complementary to the nucleotide sequence thereof can be utilized as a therapeutic agent for a disease caused by an abnormality of mast cells or mesenchymal stem cells and the like, or a disease caused by a cell proliferation abnormality, by controlling the expression of a target gene thereof or a nucleic acid of the present invention. A nucleic acid of the present invention can also be utilized as a diagnostic reagent for a disease caused by an abnormality of mast cells or mesenchymal stem cells and the like, or a disease caused by a cell proliferation abnormality, by quantifying, or detecting a mutation of, a nucleic acid, such as a micro-RNA or a micro-RNA, of the present invention.

As mast cell abnormalities, abnormalities of mast cell differentiation and degranulation, inflammatory mediator production, cytokine production, chemokine production and the like can be mentioned; as diseases caused thereby, atopic dermatitis, asthma, chronic obstructive lung disease, allergic disease and the like can be mentioned. As abnormalities of mesenchymal stem cells, an abnormality of proliferation or differentiation and the like can be mentioned; as diseases caused thereby, cancers, dysosteogenesis, achondroplasia, diabetes and the like can be mentioned. As diseases caused by a cell proliferation abnormality, cancers and diseases caused by abnormal proliferation of cells, tissue hyperplasia and the like, such as arteriosclerosis, rheumatoid arthritis, prostatic hyperplasia, blood vessel restenosis after percutaneous transvascular coronary angioplasty, fibroid lung, glomerulonephritis, and autoimmune diseases can be mentioned.

Referring to specific examples of therapeutic agents or diagnostic reagents, the micro-RNA of any one of SEQ ID NOs:1 to 1336 or the micro-RNA precursor of any one of SEQ ID NOs:1337 to 2851, and a nucleic acid having a nucleotide sequence complementary to the nucleotide sequence thereof can be used as a diagnostic reagent or a therapeutic agent for a disease caused by a mast cell abnormality. The micro-RNA of any one of SEQ ID NOs:1, 8, 21 and 36 or the micro-RNA precursor of any one of SEQ ID NOs:1337, 1352, 1372 and 1390, and a nucleic acid having a nucleotide sequence complementary to the nucleotide sequence thereof can be used as a diagnostic reagent or a therapeutic agent for a disease caused by an abnormality of mesenchymal stem cell proliferation and/or differentiation. Furthermore, the micro-RNA of any one of SEQ ID NOs:1, 3, 8, 20, 21, 22, 32 and 36 or the micro-RNA precursor of any one of SEQ ID NOs:1337, 1339, 1352, 1371, 1372, 1373, 1386 and 1390, and a nucleic acid having a nucleotide sequence complementary to the nucleotide sequence thereof can be used as a diagnostic reagent or a therapeutic agent for a disease caused by a cell proliferation abnormality.

A diagnostic reagent comprising a nucleic acid of the present invention, according to the desired diagnostic method, may comprise reagents necessary for quantitation or detection of a mutation of a nucleic acid such as micro-RNA and micro-RNA precursor of the present invention, and the like, for example, buffering agents, salts, reaction enzymes, labeled proteins that bind to a nucleic acid such as micro-RNA and micro-RNA precursor of the present invention, and the like, and a color developer for detection and the like.

Although a pharmaceutical containing a nucleic acid of the present invention or a nucleic acid, having a nucleotide sequence complementary to the nucleotide sequence thereof as an active ingredient can be administered alone, the same is normally desirably administered as a pharmaceutical preparation produced by an optionally chosen method known well in the technical field of pharmaceutical making with one or more pharmacologically acceptable carriers blended therein.

The route of administration used is desirably the most effective one in treatment; oral administration, or parenteral administration such as intraoral administration, airway administration, intrarectal administration, subcutaneous administration, intramuscular administration and intravenous administration can be mentioned, and desirably intravenous administration can be mentioned.

As dosage forms, sprays, capsules, tablets, granules, syrups, emulsions, suppositories, injection formulations, ointments, tapes and the like can be mentioned.

As preparations appropriate for oral administration, emulsions, syrups, capsules, tablets, powders, granules and the like can be mentioned.

Liquid preparations like emulsions and syrups can be produced using water, saccharides such as sucrose, sorbitol, and fructose, glycols such as polyethylene glycol and propylene glycol, oils such as sesame oil, olive oil, and soybean oil, antiseptics such as p-hydroxybenzoic acid esters, flavors such as strawberry flavor and peppermint and the like as additives.

Capsules, tablets, powders, granules and the like can be produced using excipients such as lactose, glucose, sucrose, and mannitol, disintegrants such as starch and sodium alginate, lubricants such as magnesium stearate and talc, binders such as polyvinyl alcohol, hydroxypropylcellulose, and gelatin, surfactants such as fatty acid esters, plasticizers such as glycerin and the like as additives.

As appropriate preparations for parenteral administration, injection formulations, suppositories, sprays and the like can be mentioned.

An injection formulation is prepared using a carrier consisting of a salt solution, a glucose solution or a mixture of both and the like. A suppository is prepared using a carrier such as cacao butter, hydrogenated fat or carboxylic acid. A spray is prepared using a carrier that does not stimulate the recipient's oral cavity and airway mucosa, and that disperses the active ingredients as fine particles to facilitate the absorption thereof, and the like.

As examples of the carrier, specifically, lactose, glycerin and the like can be exemplified. Depending on the nature of the nucleic acid of the present invention, and of the carrier used, preparations such as aerosols and dry powders are possible. In these parenteral preparations, components exemplified as additives for oral preparations can be added.

The dose or frequency of administration varies depending on desired therapeutic effect, method of administration, duration of treatment, age, body weight and the like, and is normally 10 μg/kg to 20 mg/kg per day for an adult.

A therapeutic agent comprising a nucleic acid of the present invention or a nucleic acid having a nucleotide sequence complementary to the nucleotide sequence thereof as an active ingredient can also be produced by blending a vector that expresses the nucleic acids and a base used in a nucleic acid therapeutic agent [Nature Genet., 8, 42(1994)].

The base used in the nucleic acid therapeutic agent may be any base for ordinary use in injection formulations; distilled water, solutions of salts such as sodium chloride or a mixture of sodium chloride and an inorganic salt, solutions of mannitol, lactose, dextran, and glucose, solutions of amino acids such as glycine and arginine, mixed solutions of organic acid solutions or salt solutions and glucose solution and the like can be mentioned. In accordance with a conventional method, using auxiliary agents such as an osmotic pressure regulator, a pH regulator, a vegetable oil such as sesame oil or soybean oil, lecithin, and a surfactant in these bases, an injection formulation may be prepared as a solution, suspension, or dispersion. These injection formulations can also be prepared as preparations for dissolution before use, by procedures such as powdering and lyophilization. A therapeutic agent of the present invention can be used for treatment as is in the case of a liquid, or after being dissolved in a base described above, optionally sterilized, in the case of a solid, just before treatment.

As a vector encoding a nucleic acid of the present invention, the recombinant viral vector prepared in 6 above can be mentioned, more specifically, retrovirus vector and lentivirus vector and the like can be mentioned.

For example, by combining a vector encoding a nucleic acid of the present invention with a polylysine-conjugated antibody that is specific for adenovirus hexon protein to prepare a complex, and binding the complex obtained to an adenovirus vector, a viral vector can be prepared. The viral vector is capable of stably reaching the desired cell, being incorporated into cells by endosome, being decomposed in the cells, and efficiently expressing the nucleic acid.

A viral vector based on Sendai virus, which is a (−) strand RNA virus, has been developed (WO97/16538, WO97/16539), and a Sendai virus incorporating a nucleic acid such as micro-RNA and micro-RNA precursor of the present invention and the like can be prepared using the Sendai virus.

A nucleic acid of the present invention and a vector encoding the same can also be migrated by a non-viral nucleic acid migration method. The same can be migrated by, for example, calcium phosphate co-precipitation [Virology, 52, 456-467 (1973); Science, 209, 1414-1422 (1980)], microinjection method [Proc. Natl. Acad. Sci. USA, 77, 5399-5403 (1980); Proc. Natl. Acad. Sci. USA, 77, 7380-7384 (1980); Cell, 27, 223-231 (1981); Nature, 294, 92-94 (1981)], membrane fusion-mediated migration mediated by liposome [Proc. Natl. Acad. Sci. USA, 84, 7413-7417 (1987); Biochemistry, 28, 9508-9514 (1989); J. Biol. Chem., 264, 12126-12129 (1989); Hum. Gene Ther., 3, 267-275, (1992); Science, 249, 1285-1288 (1990); Circulation, 83, 2007-2011 (1992)] or direct DNA uptake and receptor-mediated DNA migration method [Science, 247, 1465-1468 (1990); J. Biol. Chem., 266, 14338-14342 (1991); Proc. Natl. Acad. Sci. USA, 87, 3655-3659 (1991); J. Biol. Chem., 264, 16985-16987 (1989); BioTechniques, 11, 474-485 (1991); Proc. Natl. Acad. Sci. USA, 87, 3410-3414 (1990); Proc. Natl. Acad. Sci. USA, 88, 4255-4259 (1991); Proc. Natl. Acad. Sci. USA, 87, 4033-4037 (1990); Proc. Natl. Acad. Sci. USA, 88, 8850-8854 (1991); Hum. Gene Ther., 3, 147-154 (1991)] and the like.

Membrane fusion-mediated migration mediated by liposome allows a nucleic acid of the present invention and a vector encoding the same to be incorporated locally in the tissue, and to be expressed, by administering a liposome preparation directly to the target tissue [Hum. Gene Ther., 3, 399 (1992)]. For direct targeting of a DNA to a focus, direct DNA uptake technology is preferable.

For receptor-mediated DNA transfer, for example, a method performed by binding a DNA (usually assuming the form of a covalently cyclized supercoiled plasmid) to a protein ligand via polylysine can be mentioned. A ligand is chosen on the basis of the presence of a corresponding ligand receptor on the cell surface of the desired cell or tissue. The ligand-DNA conjugate can be injected directly into a blood vessel as desired, and can be directed to a target tissue wherein receptor binding and DNA-protein complex internalization occur. To prevent the destruction of the DNA in a cell, an adenovirus may be infected simultaneously to destroy the endosome function.

### 12. Methods of Measuring the Degree of Mast Cell Activation

The fact that a certain nucleic acid exhibits at least one of the actions to suppress activation, suppress degranulation, suppress inflammatory mediator production, suppress cytokine production and suppress chemokine production on mast cells can be confirmed by, for example, introducing a nucleic acid, such as a micro-RNA or a micro-RNA, of the present invention, or an antisense or siRNA against a target gene of the micro-RNA, into mast cells, and culturing the cells with the addition of IgE, thereafter activating human mast cells by the addition of an anti-IgE antibody and the like, measuring a released substance such as (i) histamine or β-hexosaminidase, which can serve as an index of degranulation, (ii) an inflammatory mediator such as LTC4, LTD4, LTE4, or PGD2, (iii) a cytokine such as TNF-α or GM-CSF, (iv) a chemokine such as IL-8, I-309, or MIP-1α, or the like, and comparing the result with that obtained when nothing is introduced. As described above, mast cell activation can also be examined by measuring, in place of degranulation, production of cytokines such as TNF-α and GM-CSF, production of chemokines such as IL-8, I-309, and MIP-la, production of inflammatory mediators such as LTC4, LTD4, LTE4, and PGD2, and the like [Blood 100, 3861(2002)].

The fact that a nucleic acid, such as a micro-RNA or a micro-RNA precursor, of the present invention, or an antisense, siRNA or the like against a target gene of the micro-RNA possess apoptosis inducing action can be detected by introducing them into mast cells, and performing a measurement of the fragmentation of chromatin DNA or a measurement by the TUNEL method and the like.

13. Methods of Proliferation, Differentiation into Osteoblasts and Evaluation of Mesenchymal Stem Cells

The method of acquiring mesenchymal stem cells is not particularly limited, as far as it ensures safe and efficient acquirement; as an example of a method of acquirement from human bone marrow, the method described in S. E. Haynesworth et al. Bone, 13, 81 (1992) can be mentioned.

In the sternal bone or iliac bone, the skin at the site for bone marrow paracentesis is disinfected, and particularly the subperiosteal region is fully anesthetized topically. The inner cylinder of the bone marrow paracentesis needle is removed, a 10-ml syringe containing 5,000 units of heparin is attached, and a necessary volume, usually 10 ml to 20 ml, of bone marrow fluid is aspirated. The bone marrow paracentesis needle is detached, and compressive hemostasis is performed for about 10 minutes. The bone marrow fluid acquired is centrifuged at 1,000×g, and bone marrow cells are recovered, thereafter the bone marrow cells are washed with phosphate buffer solution (phosphate-buffered saline) (PBS). After centrifugation and washing are repeated in 2 cycles, the bone marrow cells are suspended in a medium for cell culture such as α-MEM (α-modified MEM), DMEM (Dulbecco's modified MEM) or IMDM (Isocove's modified Dulbecco's medium), containing 10% fetal bovine serum (FBS), whereby a bone marrow cell fluid is obtained. The method of isolating mesenchymal stem cells from the bone marrow cell fluid is not particularly limited, as far as it allows the removal of other cells that are also present in the bone marrow cell fluid, for example, corpuscular cells, hematopoietic stem cells, vascular stem cells, fibroblasts and the like; for example, the method described in M. F. Pittenger et al. Science, 284, 143-147 (1999) can be mentioned. The bone marrow cell fluid is overlaid on Percoll having a density of 1.073 g/ml, and then centrifuged at 1,100×g for 30 minutes, whereby the cells at the interface can be isolated as mesenchymal stem cells. Also, an equal volume of Percoll diluted to 9/10 by the addition of a 10-fold concentration of PBS is added to, and mixed with, the bone marrow cell fluid, after which the mixture is centrifuged at 20,000×g for 30 minutes, whereby the cells in a fraction having a density of 1.075 to 1.060 can be isolated as mesenchymal stem cells.

Mesenchymal stem cells derived from human bone marrow can also be purchased from Cambrex and Takara Bio Inc.

As an example of a method of acquiring mesenchymal stem cells from the umbilical cord, the method described in Stem Cells, 21, 105-110 (2003) can be mentioned. A cannula is inserted into each end of the umbilical vein, which is washed with an appropriate buffer solution, for example, EBSS (Earle's balanced salt solution). An antibiotic is added to a 199 medium containing a protease, for example, 0.1% collagenase, and this is injected into the blood vessel, and incubated at 4 to 40° C., preferably at 37° C., for 1 to 60 minutes. The blood vessel is washed with EBSS, and the umbilical cord is gently massaged, after which a suspension of endothelial cells and subendothelial cells is recovered. The suspension is centrifuged at 600×g for 10 minutes, and the cells obtained are suspended in, for example, a medium prepared by adding 20 mM HEPES, 100 units/ml penicillin, 100 μg/ml streptomycin, 2 mM L-glutamine, 1 mM sodium pyruvate and 10% FBS to a DMEM medium containing a low concentration of glucose (DMEM-LG, Gibco). The cell density is adjusted to 10² to 10⁶ cells/cm², and the cells are inoculated to a culture flask and cultured under the conditions of 37° C. and 5% CO₂. While renewing the medium every 1 to 7 days, the cultivation is continued for 1 to 3 weeks, whereby mesenchymal stem cells can be acquired.

As an example of a method of acquiring mesenchymal stem cells from the endometrium, the method described in Am. J. Pathol., 163, 2259-2269 (2003) can be mentioned. Human endometrial tissue extirpated by surgical operation is shredded, and cultured with a medium that allows cell culture, preferably a medium prepared by adding 1 to 20% animal-derived serum, preferably 5 to 10% FBS, to α-MEM, DMEM, IMDM and the like. The medium may be supplemented with antibiotics such as penicillin and streptomycin. Furthermore, to facilitate cell separation, a collagen-decomposing enzyme such as type 3 collagenase and a DNase such as deoxyribonuclease I are added to the medium, and the medium is gently shaken at 20 to 40° C., preferably at 37° C., for 10 minutes to 5 hours, preferably for 1 hour. Each endometrial gland is separated while examining microscopically, and cultured in an appropriate culture vessel, for example, a 24-well culture dish, under the conditions of 37° C. and 5% CO₂, whereby mesenchymal stem cells can be acquired.

As an example of a method of acquiring mesenchymal stem cells from a tooth, a dental germ, or periodontal tissue, the methods described in Lancet, 364, 149-155(2004), Proc. Natl. Acad. Sci. USA, 97, 13625-13630 (2000) can be mentioned. The human tooth used may be any of deciduous teeth and permanent teeth such as incisor teeth, canine teeth, premolar teeth, and molar teeth. For example, the periodontal ligament is carefully separated from the surface of the root of a third molar (wisdom tooth) extracted, and a digestive reaction is carried out using a protease such as collagenase, trypsin, pronase, elastase, dispase, or hyaluronidase at 37° C. for 1 hour. The tissue residue is removed using a strainer, a mesh, a filter and the like, whereby mesenchymal stem cells can be acquired. Mesenchymal stem cells can also be acquired by washing the surface of an extracted third molar with PBS and the like, thereafter cutting the joint of the cement and the enamel to expose the pulp, carefully separating the dental pulp tissue from the dental crown and root, and treating the dental pulp tissue with a protease as described above, thereafter removing the tissue residue.

As methods of mesenchymal stem cell isolation other than those described above, methods can be mentioned wherein mesenchymal stem cells are isolated using a surface antigen expressed in mesenchymal stem cells or a reporter vector having a promoter and enhancer of a gene that is specific for mesenchymal stem cells. Specifically, a method of isolating stem cells using the AC133 antigen (U.S. Pat. No. 6,468,794), methods of isolating mesenchymal stem cells using a reporter vector having a promoter and enhancer of the Sox gene (US2002/0135539), the Nestin gene or the Musashi gene (JP-A-2002-034580), and the like can be mentioned.

Stem cells can also be separated using a method wherein FACS fractionation with the potential for extracellular discharge of Hoechst33342 as an index is used to concentrate stem cells in a side population (SP) [Journal of Experimental Medicine, 183, 1797-806 (1996)]. A method wherein SH2-positive, SH4-positive, CD29-positive, CD44-positive, CD71-positive, CD90-positive, CD106-positive, CD120a-positive, CD124-positive, CD14-negative, CD34-negative, and CD45-negative cells are separated as mesenchymal stem cells using a cell sorter or magnetic beads [Science, 284, 143-147 (1999)] can also be used.

As examples of media used to culture mesenchymal stem cells, the media for cell culture described in “Soshiki Baiyou No Gijyutsu, Kiso-hen, 3rd edition”, Asakura Shoten (1996) and the like can be mentioned; media for cell culture such as α-MEM, DMEM, and IMDM, supplemented with 1 to 20% of a serum such as bovine or human serum, are preferably used. Although the culture conditions may be any conditions that allow cultivation of the cells, culturing temperature is preferably 33 to 37° C., and the cultivation is preferably performed in an incubator filled with 5 to 10% gaseous CO₂. Mesenchymal stem cells are preferably proliferated in adhesion to a plastic culture dish for ordinary tissue culture. When the cells have proliferated over the entire surface of the culture dish, the medium is removed, and a trypsin-EDTA solution is added to suspend the cells. The cells suspended are washed with PBS or a medium for cell culture, after which the cells are 2 fold to 20 fold diluted with a medium for cell culture, and sown to a new culture dish, whereby further subculture can be performed.

Whether a certain nucleic acid controls the proliferation of mesenchymal stem cells can be confirmed by, for example, introducing a nucleic acid, such as a micro-RNA or a micro-RNA precursor, of the present invention, or an antisense, siRNA or the like against a target gene of the micro-RNA, into the mesenchymal stem cells, and comparing the degree of cell proliferation with that of a negative control. A method of measuring the degree of cell proliferation can be performed by the method described in 14 below.

As a method of examining the influence on the process of differentiation from mesenchymal stem cells to osteoblasts, for example, confirmation is made as described below. Specifically, under conditions that induce differentiation from mesenchymal stem cells to osteoblasts, a nucleic acid, micro-RNA or micro-RNA precursor of the present invention, or an antisense, siRNA or the like against a target gene of the micro-RNA, is introduced into the mesenchymal stem cells, and the cells are cultured. Genes or proteins whose expression increased with the progression of differentiation into osteoblasts are analyzed, and compared with negative control.

As a method of inducing differentiation from mesenchymal stem cells to osteoblasts, any method can be used, as far as it enables induction of differentiation from mesenchymal stem cells to osteoblasts; for example, a method described in Science, 284, 143-147 (1999) can be mentioned. Specifically, by sowing mesenchymal stem cells to an incubator, and thereafter continuing to culture the cells in a medium for cell culture containing dexamethasone, ascorbic acid-diphosphate, and β-glycerophosphate for 1 to 4 weeks, mesenchymal stem cells can be differentiated into osteoblasts.

As quantitative analytical methods for genes whose expression increases as a result of differentiation into osteoblasts, analysis by RT-PCR (reverse transcription-polymerase chain reaction), Northern blot analysis, dot blot hybridization, DNA microarray and the like can be mentioned.

As quantitative analytical methods for proteins whose expression increases as a result of differentiation into osteoblasts, Western blot analysis, immunohistological staining, ELISA and the like using an antibody that specifically reacts on the protein can be mentioned.

As genes or proteins whose expression increases as a result of differentiation into osteoblasts, type I collagen, osteocalcin, osteonectin, osteopontin, bone sialoprotein, Runx2 (runt-related gene 2), alkaline phosphatase (ALP) and the like can be mentioned.

As methods of evaluating the degree of differentiation into osteoblasts, staining cells by means of the ALP enzyme activity in the osteoblasts, and a method wherein ALP enzyme activity is measured can be mentioned. More specifically, as a method of such cell staining, a method can be mentioned wherein the alcohol moiety of the phosphoric acid ester of the substrate hydrolyzed by ALP enzyme in osteoblasts is coupled with a diazonium salt, and precipitated with azo dye in the enzyme activity portion. As an example of the substrate, Naphthol AS-MX phosphate can be mentioned; as an example of the azo dye, Fast Violet Blue can be mentioned. A kit comprising the same, for example, leukocyte alkaline phosphatase (manufactured by Sigma) and the like may be used. As kits for measuring ALP enzyme activity, for example, Alkaline Phospha B-Test Wako (manufactured by Wako Pure Chemical Industries, Ltd.) and the like may be used.

Furthermore, by detecting a calcified component produced by osteoblasts, differentiation into osteoblasts can also be confirmed. As methods of detecting a calcified component, staining methods such as von Kossa staining and Alizarin Red staining can be mentioned.

Von Kossa staining is a method of detecting calcium phosphate, a calcified component, using silver nitrate. Specifically, a 1 to 5% aqueous solution of silver nitrate is reacted with cells fixed with paraffin and the like and exposed to light, and the portion that develops a black color in which calcium phosphate is present is quantified by, for example, measuring the area where the color is developed, whereby the degree of differentiation into osteoblasts can be evaluated.

Alizarin Red staining is a method based on the fact that Alizarin Red S exhibits specific binding to calcium to form a lake. Specifically, 0.01 to 5% Alizarin Red S solution is reacted with cells fixed with paraffin and the like, and the portion that develops a red-purple to orange-red color is quantified by, for example, measuring the area where the color is developed, whereby the degree of differentiation into osteoblasts can be evaluated.

### 14. Method of Measuring Proliferation of Cancer Cells and Other Cells

The method of measuring cell proliferation is not particularly limited, as far as it enables a measurement of an index that reflects cell count or cell proliferation rate. Viable cell counting, DNA synthesis rate measurements, total protein content measurements and the like can be used.

As a method of evaluating viable cell counts, a method wherein the ATP content in cells is measured can be mentioned. It is known that the ATP content in cells is proportional to the number of cells in culture (J. Immunol. Meth. 160, 81-88 (1993)). As more specific methods of measuring the ATP content in cells, the MTT method, the XTT method and the like can be mentioned (J. Immunol. Meth. 65, 55-63 (1983)). A method can also be mentioned wherein ATP content is measured by luminescence of a luciferin substrate by the ATP-dependent enzyme luciferase. As a kit for measuring the ATP content in cells, for example, Cell Titer-Glo® Luminescent Cell viability Assay (manufactured by Promega) and the like may be used.

### 15. Methods of Measuring the Degree of Cell Death of Cancer Cells and Other Cells

As methods of measuring the degree of cell death, a method wherein dead cells are stained with a dye such as Propium Iodide, a method wherein the activity of an enzyme leaked extracellularly as a result of cell death is measured, and the like can be used. For the latter, for example, a method wherein the enzyme activity of extracellularly leaked adenylate kinase is measured can be utilized. More specifically, ToxiLight® Non-Destructive Cytotoxicity BioAssay Kit (manufactured by Lonza) and the like may be used.

It is also possible to measure the degree exclusively of apoptosis (programmed cell death), which is important in relation to cancers, out of the various types of cell death. As methods of evaluating cell apoptosis, a method wherein the degree of DNA fragmentation is measured, a method wherein changes in cell membrane constituent lipids are measured, and a method wherein the activity of caspase 3/7, an intracellular protease induced upon apoptosis, is measured, can be mentioned. As a more specific method of measuring caspase 3/7 activity in cells, a method wherein a luciferin substrate liberated by caspase 3/7 activity is measured by luciferin luminescence by a luciferase enzyme reaction can be mentioned. As kits for measuring caspase 3/7 activity in cells, for example, Caspase-Glo® 3/7 Assay (manufactured by Promega) and the like may be used.

Hereinafter, the present invention is described specifically by means of the following examples.

### Example 1

### Extraction of Micro-RNAs Expressed in Human Mast Cells

### (1) RNA Extraction

LAD2 is a recently established human mast cell line, and is known to well retain the nature of human mast cells [Leuk. Res., 27, 671 (2003); Leuk. Res., 27, 677 (2003)]. Hence, extraction of micro-RNAs from LAD2 was performed. LAD2 was obtained from the National Institute of Allergy and Infectious Diseases, National Institutes of Health (Bethesda, Md. 20892-1881, USA), and cultured with a Stem Pro-34 medium [manufactured by Invitrogen] containing 100 ng/mL SCF in a 37° C. 5% CO₂ concentration incubator.

### (2) Cloning of Low-Molecular RNAs

Using 200 μg of the LAD2-derived total RNA acquired in (1) above, and according to the method of Lau et al. (Science 294, 858-862, 2001), excision for low-molecular RNAs by means of 15% polyacrylamide gel electrophoresis, 5′-adenylated 3′-adapter ligation, 5′-adapter ligation, reverse transcription, PCR amplification, concatemerization, and ligation to the pCR2.1-TOPO vector were performed sequentially, to achieve cloning of the low-molecular RNAs. Next, the nucleotide sequences of the low-molecular RNAs cloned were determined. The 5′-adenylated 3′-adapter used was miRNA Cloning Linker, manufactured by Integrated DNA Technologies.

Separately from the above-described method, the nucleotide sequence was also determined by performing separation and cutting out of a low-molecular RNA by 15% polyacrylamide gel electrophoresis, 5′ terminal dephosphorylation, 3′-adapter ligation, phosphorylation, 5′-adapter ligation, reverse transcription, PCR amplification, and ligation to a microbead vector sequentially to achieve cloning of the low-molecular RNA, and reading the nucleotide sequence of the microbead, using 200 μg of the LAD2-derived total RNA acquired in (1) above, according to the method of Mineno et al. [Nucleic Acids Research, 34, 1765-1771, (2006)].

### Example 2

### Identification of Micro-RNAs

From among the low-molecular RNAs obtained in Example 1, first, those whose nucleotide sequences did not agree with any one in miRBase (http://microrna.sanger.ac.uk/), which is a database for known micro-RNAs, were selected. Surrounding genome sequences wherein DNA sequences corresponding to those nucleotide sequences were extended by about 50 nt toward the 5′ side and the 3′ side, respectively, were acquired from UCSC Genome Bioinformatics (http://genome.ucsc.edu/), and the secondary structures of the RNAs expected to be transcribed from the genome sequences were predicted using RNAfold. As a result, 1336 types were found to be novel micro-RNAs located in one chain of the hairpin structure. The nucleotide sequences thereof and the nucleotide sequences of micro-RNA precursors comprising these micro-RNAs are shown in Tables 1. The micro-RNAs having the respective nucleotide sequences were given the names KHK miR 1001 to 2344 (Table 1). If one micro-RNA can assume hairpin structures derived from genome sequences at different positions, all thereof are shown.

As an example secondary structure, the hairpin structure of KHK_miR_{—}1194 is shown in FIG. 1.

### Example 3

### Detection of Functions of Micro-RNAs

By determining whether or not a micro-RNA obtained in Example 2 undergoes processing by Dicer protein, whether or not the same functions as a micro-RNA can be determined.

Of the micro-RNAs obtained in Example 2, the micro-RNA of KHK_miR_{—}1194 can have a function thereof detected as described below. First, a single-stranded RNA having the nucleotide sequence of SEQ ID NO:1580 is synthesized, and reacted with the Dicer Enzyme attached to the X-treme GENE siRNA Dicer Kit (manufactured by Roche-Applied Science). Next, the reaction product is electrophoresed with 15% polyacrylamide gel; detection of a band 20 to 25 nucleotides in size indicates that the possession of a function as a micro-RNA.

### Example 4

Action on Degranulation of Human Mast Cells with Micro-RNA Expressed Forcibly Therein

Each micro-RNA precursor obtained in Example 2 was introduced to LAD2, a human mast cell line, to induce degranulation, and the influence of the micro-RNA precursor was examined.

The LAD2 was cultured with a Stem Pro-34 medium (manufactured by Invitrogen) containing 100 ng/mL SCF.

The LAD2 was sown to a 6-well plate at about 5×10⁵ cells per well, and a micro-RNA precursor was introduced using a lipofection method, specifically, Gene Silencer (manufactured by Genlantis), to obtain a final concentration of 30 nM. The micro-RNA (hereinafter also referred to as miRNA) precursors used were KHK_miR_{—}1001, 1002, 1003, 1008, 1014, 1020, 1021, 1022, 1025, 1032, and 1036, synthesized as Pre-miR™ miRNA Precursor Molecules by Ambion. These were chemically synthesized double-stranded nucleic acid molecules, designed to allow nucleic acids consisting of the nucleotide sequences of SEQ ID NOs:1, 2, 3, 8, 14, 20, 21, 22, 25, 32, and 36, respectively, to be incorporated by a complex similar to RISC, which is a factor for the activity of an miRNA, to exhibit the same function as the miRNA. For a sequence corresponding to the human genome sequence of SEQ ID NO:1, a nucleic acid consisting of a sequence having 1 nucleotide on the 5′ side and 4 nucleotides on the 3′ side deleted from SEQ ID NO:1 (SEQ ID NO:2852) was provided, and KHK_miR_{—}1001_{—}2, synthesized as Pre-miR™ miRNA Precursor Molecule by Ambion, was also used. For negative control, Pre-miR™ miRNA Precursor Molecules-Negative Control #2 (hereinafter referred to as miR-negacon #2) (manufactured by Ambion) was introduced into the LAD2 in the same manner. Lipofection was performed per the directions attached to the product.

Two days after introduction of the micro-RNA precursor by a lipofection method, 1 μg/mL human myeloma IgE (manufactured by Cosmo Bio Co., Ltd.) was added, and the cells were cultured in a 37° C. 5% CO₂ concentration incubator overnight. On the day that followed, the medium was removed via centrifugation, and the plate was washed with a Tyrode buffer solution (126.1 mmol/L NaCl, 4.0 mmol/L KCl, 1.0 mmol/L CaCl₂, 0.6 mmol/L MgCl₂, 0.6 mmol/L KH₂PO₄, 10 mM HEPES, 5.6 mmol/L D-glucose, 0.1% bovine serum albumin, pH 7.4), after which 3.9 mL of the Tyrode buffer solution was added to suspend the cells, and the suspension was dispensed to a 96-well plate at 100 μL per well. Next, a rabbit anti-human IgE antibody (manufactured by DAKO) was added to obtain a final concentration of 10 μg/mL, and this was followed by incubation in a 37° C. 5% CO₂ concentration incubator for 20 minutes to induce degranulation. The supernatant was recovered via centrifugation, and the β-hexosaminidase activity in the supernatant was measured, whereby the degree of degranulation was determined. The β-hexosaminidase activity was measured by adding 50 μL of 4 mmol/L p-nitrophenyl N-acetyl-β-glucosaminide (manufactured by Sigma) dissolved in 40 mmol/L citrate buffer solution (pH 4.5) to 50 μL of the supernatant recovered, and incubating the mixture at 37° C. for 1 hour, thereafter adding 100 μL of 0.2 mol/L glycine (pH 10.7), and measuring the absorbance of the sample at 405 nm using the plate reader 1420 ARVOsx (manufactured by Perkin Elmer). Also, by performing the same experiment but with the addition of Triton X-100 at a final concentration of 1% in place of the rabbit anti-human IgE antibody, the total β-hexosaminidase activity in LAD2 was measured. The ratio of degranulation was calculated as the ratio (%) of the β-hexosaminidase activity in the supernatant relative to total β-hexosaminidase activity; taking the degranulation ratio of a control plot (Gene Silencer only) as 1.0, the relative degranulation activity of each was calculated. The results are shown in Table 3.

TABLE 3

| introduced micro-RNA | relative degranulation |
|---|---|
| precursor | activity |
| KHK_miR_1001 | 1.34 |
| KHK_miR_1001_2 | 1.25 |
| KHK_miR_1002 | 1.80 |
| KHK_miR_1003 | 1.42 |
| KHK_miR_1008 | 1.33 |
| KHK_miR_1014 | 1.38 |
| KHK_miR_1020 | 1.31 |
| KHK_miR_1022 | 1.25 |
| KHK_miR_1025 | 1.24 |
| KHK_miR_1032 | 1.53 |
| KHK_miR_1036 | 1.43 |
| miR-negacon#2 | 1.01 |

As a result, it was found that with introduction of KHK_miR_{—}1001, 1001_{—}2, 1002, 1003, 1008, 1014, 1020, 1022, 1025, 1032, or 1036, the degranulation of LAD2 stimulated by IgE receptor is promoted.

### Example 5

Proliferation Activity, Osteoblast Differentiation, and Viable Cell Count of Mesenchymal Stem Cells with Micro-RNAs Expressed Forcibly Therein

Each micro-RNA precursor obtained in Example 2 was introduced into human mesenchymal stem cells (hereinafter also referred to as hMSCs), and the effects of the micro-RNA precursors on the proliferation and osteoblast differentiation were examined.

The human mesenchymal stem cells were obtained from Cambrex, and cultured with an IMDM medium (manufactured by Invitrogen) containing 20% fetal bovine serum (FBS) (manufactured by JRH Bioscience) in a 37° C. 5% CO₂ concentration incubator. The hMSCs were sown to a 24-well plate at about 6.2×10³ cells per well, and cultured with the IMDM medium containing 20% FBS overnight. One day later, the micro-RNA precursor was introduced into the hMSCs using a lipofection method, specifically, Lipofectamine 2000 (manufactured by Invitrogen), to obtain a final concentration of 20 nM. The micro-RNA precursors used were KHK_miR_{—}1001, 1001_{—}2, 1002, 1003, 1008, 1014, 1020, 1021, 1022, 1025, 1032, and 1036, synthesized as Pre-miR™ miRNA Precursor Molecules by Ambion. Lipofection was performed per the directions attached to the product.

On the day after introduction of the micro-RNA precursor by the lipofection method, the medium was replaced with an osteoblast differentiation induction medium [an IMDM medium containing 20% FBS, supplemented with 0.1 μmol/L dexamethasone, 50 μmol/L ascorbic acid-diphosphoric acid (manufactured by Sigma), and 10 mmol/L β-glycerophosphite (manufactured by Sigma)], and cultivation was continued with renewal of the osteoblast differentiation induction medium at a frequency of once per 3 days.

Two weeks after the start of the cultivation, cell morphology was examined under a phase-contrast microscope (manufactured by Nikon), and alkaline phosphatase staining was performed, to detect osteoblasts. Specifically, first, the cells were once washed with phosphate buffer solution (hereinafter also referred to as PBS (phosphate-buffered saline)) (manufactured by Invitrogen), and fixed with a fixative solution (10% formalin/PBS) for 5 minutes. The cells were washed with distilled water, and thereafter reacted with a mixed solution of Naphthol AS-MX phosphate (manufactured by Sigma) and Fast Violet B solution (manufactured by Sigma) in the dark for 30 minutes to cause an alkaline phosphatase reaction. Furthermore, the cells were washed with distilled water, and osteoblasts stained red were examined under a phase contrast microscope and photographed using a digital camera (manufactured by Nikon).

As a result, it was found that the hMSCs incorporating Pre-miR™ miRNA Precursor Molecules of KHK_miR_{—}1008, 1021, or 1036 had fewer cells than the hMSCs not incorporating any micro-RNA precursor, and also had fewer positive cells stained with alkaline phosphatase. Micro-RNA precursors are converted to micro-RNAs in cells; hence, it was found that the micro-RNAs derived from these precursors exhibit a suppressive activity on the proliferation of hMSCs and suppressive activity on the differentiation thereof into osteoblasts. Conversely, it was found that the hMSCs incorporating Pre-miR™ miRNA Precursor Molecules of KHK_miR_{—}1001 or 1001_{—}2 had a larger number of positive cells stained with alkaline phosphatase than the hMSCs not incorporating any micro-RNA precursor. From this, it was found that the micro-RNAs derived from these precursors exhibit a promotive activity on the differentiation of hMSCs into osteoblasts.

Four days after introduction of each micro-RNA precursor by the lipofection method, viable cell ratios were measured using CellTiter-Glo™ Luminescent Cell Viability Assay (manufactured by Promega). The results are shown in Table 4. As shown in Table 4, it was found that with introduction of KHK_miR_{—}1001, the viable cell ratio increased than usual.

TABLE 4

| introduced micro-RNA precursor | viable cell ratio |
|---|---|
| KHK_miR_1001 | 1.28 |
| miR_negacon#2 | 1.00 |

### Example 6

Viable Cell Ratio and Apoptotic Activity in Colon Cancer-Derived Cell Line with Micro-RNA Expressed Forcibly Therein

Each micro-RNA precursor obtained in Example 2 was introduced to a colon cancer-derived cell line, and the effects of the micro-RNA precursor on viable cell ratio and apoptotic activity were examined.

The DLD-1 human colorectal cancer-derived cell line (hereinafter to be sometimes referred to as DLD-1) was obtained from the American Type Culture Collection (ATCC) (hereinafter referred to as ATCC) (ATCC CCL-221). DLD-1 was cultured with an RPMI1640 medium (manufactured by Invitrogen) containing 10% fetal bovine serum (FBS) (manufactured by JRH Biosciences) in a 37° C. 5% CO₂ concentration incubator.

DLD-1 was sown to a 96-well plate at about 2500 cells per well, and cultured in an RPMI medium containing 10% FBS overnight. After 1 day, a micro-RNA precursor was introduced to the DLD-1 using a lipofection method, specifically Lipofectamine 2000 (manufactured by Invitrogen), to obtain a final concentration of 5 nM or 25 nM. The micro-RNA precursors used were KHK_miR_{—}1001, 1001_{—}2, 1002, 1003, 1008, 1014, 1020, 1021, 1022, 1025, 1032, and 1036, synthesized as Pre-miR™ miRNA Precursor Molecules by Ambion. In addition, miR-negacon #2 (manufactured by Ambion) was also introduced to DLD-1, and this was used as the negative control. Lipofection was performed per the directions attached to the product.

Three days after introduction of the micro-RNA precursor by a lipofection method, viable cell ratios were measured using CellTiter-Glo™ Luminescent Cell Viability Assay (manufactured by Promega). Taking the viable cell ratio of DLD-1 in a control plot (Lipofectamine 2000 only) as 1.0, the relative viable cell ratio of each was calculated. As a result, as shown in Table 5, with introduction of KHK_miR_{—}1001, 1001_{—}2, 1032, or 1036, a decrease of 40% or more in viable cell ratio was observed.

TABLE 5

| introduced micro- | viable cell ratio | viable cell ratio |
|---|---|---|
| RNA precursor | (5 nM) | (25 nM) |
| KHK_miR_1001 | 0.23 | 0.11 |
| KHK_miR_1001_2 | 0.32 | 0.19 |
| KHK_miR_1032 | 0.58 | 0.59 |
| KHK_miR_1036 | 0.70 | 0.46 |
| miR_negacon#2 | 0.87 | 0.87 |

Two days after introduction of the micro-RNA precursor by a lipofection method, caspase 3/7 activity was measured using Caspase-Glo® 3/7 assay (manufactured by Promega) per the directions attached to the product. Taking the caspase 3/7 activity value of DLD-1 in a control plot (Lipofectamine 2000 only) as 1.0, the relative caspase 3/7 activity value of each was calculated. The results are shown in Table 6. As shown in Table 6, with introduction of KHK_miR_{—}1001, 1001_{—}2, or 1036, an increase of 50% or more in caspase 3/7 activity was observed.

TABLE 6

| introduced micro-RNA precursor | caspase 3/7 activity |
|---|---|
| KHK_miR_1036 | 2.50 |
| KHK_miR_1001 | 2.07 |
| KHK_miR_1001_2 | 1.84 |
| miR_negacon#2 | 0.85 |

### Example 7

Viable Cell Ratio in Ovarian Cancer-Derived Cell Line with Micro-RNA Expressed Forcibly Therein

Each micro-RNA precursor obtained in Example 2 was introduced to an ovarian cancer-derived cell line, and the effects of the micro-RNA precursor on viable cell ratio was examined.

The A2780 human ovarian cancer-derived cell line (Nature, 295, 116-119 (1982); Science, 224, 994-996 (1984); Semin. Oncol., 11, 285-298 (1984); hereinafter to be referred to as A2780) was cultured with an RPMI1640 medium (manufactured by Invitrogen) containing 5% FBS (manufactured by JRH Biosciences) in a 37° C. 5% CO₂ concentration incubator.

A2780 was sown to a 96-well plate at about 2500 cells per well, and cultured with an RPMI medium containing 10% FBS overnight. After 1 day, the micro-RNA precursor was introduced to A2780 using a lipofection method, specifically Lipofectamine 2000 (manufactured by Invitrogen), to obtain a final concentration of 5 nM or 25 nM. The micro-RNA precursors used were KHK_miR_{—}1001, 1001_{—}2, 1002, 1003, 1008, 1014, 1020, 1021, 1022, 1025, 1032, and 1036, synthesized as Pre-miR™ miRNA Precursor Molecules by Ambion. miR-negacon #2 (manufactured by Ambion) was also introduced into A2780, and this was used for negative control.

Three days after introduction of the micro-RNA precursor by a lipofection method, viable cell ratios were measured using CellTiter-Glo™ Luminescent Cell Viability Assay (manufactured by Promega). Taking the viable cell ratio of A2780 in a control plot (Lipofectamine 2000 only) as 1.0, the relative viable cell ratio of each was calculated. The results are shown in Table 7. As shown in Table 7, with introduction of KHK_miR_{—}1003, 1008, 1020, 1021, 1022, 1032, or 1036, a decrease of 40% or more in viable cell ratio was observed.

TABLE 7

| introduced micro- | viable cell ratio | viable cell ratio |
|---|---|---|
| RNA precursor | (5 nM) | (25 nM) |
| KHK_miR_1032 | 0.36 | 0.43 |
| KHK_miR_1003 | 0.41 | 0.45 |
| KHK_miR_1008 | 0.43 | 0.43 |
| KHK_miR_1036 | 0.44 | 0.36 |
| KHK_miR_1021 | 0.46 | 0.64 |
| KHK_miR_1020 | 0.48 | 0.57 |
| KHK_miR_1022 | 0.52 | 0.52 |
| miR_negacon#2 | 1.00 | 1.05 |

### INDUSTRIAL APPLICABILITY

The present invention provides a nucleic acid such as a micro-RNA or a micro-RNA precursor, having a novel sequence. The nucleic acid of the present invention makes it possible to detect the expression or a mutation of a micro-RNA, to separate cells, to suppress the expression of a target sequence gene, to screen for a substance that promotes or suppresses a function of a micro-RNA, and to diagnose or treat a disease caused by a mast cell abnormality, a disease caused by an abnormality of mesenchymal stem cell proliferation or differentiation, cancers, and a disease caused by abnormal proliferation of cells, tissue hyperplasia or the like.

1.-64. (canceled)

65. An isolated nucleic acid that
(a) comprises a nucleotide sequence having an identity of 90% or more to the nucleotide sequence of any one of SEQ ID NOs: 1 to 1336, or
(b) hybridizes under stringent conditions with a strand complementary to a nucleic acid consisting of the nucleotide sequence of any one of SEQ ID NOs: 1 to 1336.

66. The isolated nucleic acid of claim 65, wherein the nucleic acid consists of the nucleotide sequence of any one of SEQ ID NOs: 1 to 1336.

67. An isolated nucleic acid that
(a) consists of a nucleotide sequence having an identity of 80% or more to the nucleotide sequence of any one of SEQ ID NOs: 1337 to 2851, or
(b) hybridizes under stringent conditions with a strand complementary to a nucleic acid consisting of the nucleotide sequence of any one of SEQ ID NOs: 1337 to 2851.

68. The isolated nucleic acid of claim 67, wherein the nucleic acid consists of the nucleotide sequence of any one of SEQ ID NOs: 1337 to 2851.

69. An isolated nucleic acid consisting of a nucleotide sequence complementary to
(a) the nucleic acid of claim 65, or
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851.

70. An isolated double-stranded nucleic acid consisting of
(i) a first strand nucleic acid, which is
(a) the nucleic acid of claim 65, or
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851, and
(ii) a second strand nucleic acid consisting of a nucleotide sequence complementary to the nucleotide sequence of the first strand nucleic acid.

71. A vector comprising a nucleic acid operably linked to a promoter, wherein the nucleic acid is selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d).

72. A cell comprising
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c),
(f) a double-stranded nucleic acid consisting of the nucleic acids of (b) and the nucleic acid of (d), or
(g) a vector comprising a nucleic acid operably linked to a promoter, wherein the nucleic acid is selected from the group consisting any one of (a) to (f).

73. A composition comprising
(i) a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d), and
(ii) a carrier therefor.

74. A composition comprising
(i) a substance that promotes or suppresses the expression or function of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d), and
(ii) a carrier therefor.

75. A composition comprising
(i) a substance that suppresses the expression of a target gene of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d), and
(ii) a carrier therefor.

76. A composition comprising
(i) a nucleic acid selected from the group consisting of
(a) the nucleic acid of claim 65, wherein the SEQ ID NO is any one of 1, 8, 21, and 36,
(b) a nucleic acid that
(b1) consists of a nucleotide sequence having an identity of 80% or more to the nucleotide sequence of any one of SEQ ID NOs: 1337, 1352, 1372, and 1390, or
(b2) hybridizes under stringent conditions with a strand complementary to a nucleic acid consisting of the nucleotide sequence of any one of SEQ ID NOs: 1337, 1352, 1372, and 1390,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d), and
(ii) a carrier therefor.

77. A composition comprising
(i) a substance that promotes or suppresses
(A) the expression or function of
(a) the nucleic acid of claim 65, wherein the SEQ ID NO is any one of 1, 8, 21, and 36,
(b) a nucleic acid consisting of a nucleotide sequence having an identity of 80% or more to the nucleotide sequence of any one of SEQ ID NOs: 1337, 1352, 1372, and 1390, or
(c) a nucleic acid that hybridizes under stringent conditions with a strand complementary to a nucleic acid consisting of the nucleotide sequence of any one of SEQ ID NOs: 1337, 1352, 1372, and 1390, or
(B) the expression of a target gene of the nucleic acid of (a), (b), or (c), and
(ii) a carrier therefor.

78. A composition comprising
(i) a nucleic acid selected from the group consisting of
(a) the nucleic acid of claim 65, wherein the SEQ ID NO is any one of 1, 3, 8, 20, 21, 22, 32, and 36
(b) a nucleic acid that
(b1) consists of a nucleotide sequence having an identity of 80% or more to the nucleotide sequence of any one of SEQ ID NOs: 1337, 1339, 1352, 1371, 1372, 1373, 1386, and 1390, or
(b2) hybridizes under stringent conditions with a strand complementary to a nucleic acid consisting of the nucleotide sequence of any one of SEQ ID NOs: 1337, 1339, 1352, 1371, 1372, 1373, 1386, and 1390,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and the nucleic acid of (c), and
(f) a double-stranded nucleic acid consisting of the nucleic acid of (b) and the nucleic acid of (d), and
(ii) a carrier therefor.

79. A composition comprising
a substance that promotes or suppresses
(A) the expression or function of
(a) the nucleic acid of claim 65, wherein the SEQ ID NO is any one of 1, 3, 8, 20, 21, 22, 32, and 36,
(b) a nucleic acid consisting of a nucleotide sequence having an identity of 80% or more to the nucleotide sequence of any one of SEQ ID NOs: 1337, 1339, 1352, 1371, 1372, 1373, 1386, and 1390, or
(c) a nucleic acid that hybridizes under stringent conditions with a strand complementary to a nucleic acid consisting of the nucleotide sequence of any one of SEQ ID NOs: 1337, 1339, 1352, 1371, 1372, 1373, 1386, and 1390, or
(B) the expression of a target gene of the nucleic acid of (a), (b), or (c), and
(ii) a carrier therefor.

80. A composition comprising
(i) a nucleic acid selected from the group consisting of
(a) the nucleic acid of claim 65, wherein the SEQ ID NO is any one of 1, 2, 3, 8, 14, 20, 22, 25, 32, and 36,
(b) a nucleic acid consisting of a nucleotide sequence having an identity of 80% or more to the nucleotide sequence of any one of SEQ ID NOs: 1337, 1338, 1339, 1352, 1363, 1371, 1373, 1377, 1386, and 1390, and
(c) a nucleic acid that hybridizes under stringent conditions with a strand complementary to a nucleic acid consisting of the nucleotide sequence of any one of SEQ ID NOs: 1337, 1338, 1339, 1352, 1363, 1371, 1373, 1377, 1386, and 1390, and
(ii) a carrier therefor.

81. A composition comprising
(i) a nucleic acid consisting of a nucleotide sequence complementary to a nucleic acid selected from the group consisting of
(a) the nucleic acid of claim 65, wherein the SEQ ID NO is any one of 1, 2, 3, 8, 14, 20, 22, 25, 32, and 36,
(b) a nucleic acid consisting of a nucleotide sequence having an identity of 80% or more to the nucleotide sequence of any one of SEQ ID NOs: 1337, 1338, 1339, 1352, 1363, 1371, 1373, 1377, 1386, and 1390, and
(c) a nucleic acid that hybridizes under stringent conditions with a strand complementary to a nucleic acid consisting of the nucleotide sequence of any one of SEQ ID NOs: 1337, 1338, 1339, 1352, 1363, 1371, 1373, 1377, 1386, and 1390, and
(ii) a carrier therefor.

82. A composition comprising
(i) a double-stranded nucleic acid consisting of a
(A) a first strand nucleic acid selected from the group consisting of
(a) the nucleic acid of claim 65, wherein the SEQ ID NO is any one of 1, 2, 3, 8, 14, 20, 22, 25, 32, and 36,
(b) a nucleic acid consisting of a nucleotide sequence having an identity of 80% or more to the nucleotide sequence of any one of SEQ ID NOs: 1337, 1338, 1339, 1352, 1363, 1371, 1373, 1377, 1386, and 1390, and
(c) a nucleic acid that hybridizes under stringent conditions with a strand complementary to a nucleic acid consisting of the nucleotide sequence of any one of SEQ ID NOs: 1337, 1338, 1339, 1352, 1363, 1371, 1373, 1377, 1386, and 1390, and
(B) a second strand nucleic acid consisting of a nucleotide sequence complementary to the nucleotide sequence of the first strand nucleic acid, and
(ii) a carrier therefor.

83. A composition comprising
(i) a substance that promotes or suppresses
(A) the expression or function of a nucleic acid selected from the group consisting of
(a) the nucleic acid of claim 65, wherein the SEQ ID NO is any one of 1, 2, 3, 8, 14, 20, 22, 25, 32, and 36,
(b) a nucleic acid consisting of a nucleotide sequence having an identity of 80% or more to the nucleotide sequence of any one of SEQ ID NOs: 1337, 1338, 1339, 1352, 1363, 1371, 1373, 1377, 1386, and 1390, and
(c) a nucleic acid that hybridizes under stringent conditions with a strand complementary to a nucleic acid consisting of the nucleotide sequence of any one of SEQ ID NOs: 1337, 1338, 1339, 1352, 1363, 1371, 1373, 1377, 1386, and 1390, or
(B) the expression of a target gene of the nucleic acid of (a), (b), or (c), and
(ii) a carrier therefor.

84. A composition comprising
(i) a nucleic acid selected from the group consisting of
(a) the nucleic acid of claim 65, wherein the SEQ ID NO is 1,
(b) a nucleic acid that
(b1) consists of a nucleotide sequence having an identity of 80% or more to the nucleotide sequence of SEQ ID NO: 1337, or
(b2) hybridizes under stringent conditions with a strand complementary to a nucleic acid consisting of the nucleotide sequence of SEQ ID NO: 1337,
(c) a nucleic acid consisting of a nucleotide sequence complementary to the nucleic acid of claim 65, wherein the SEQ ID NO is any one of 8, 21, and 36, and
(d) a nucleic acid consisting of a nucleotide sequence complementary to a nucleic acid that
(d1) consists of a nucleotide sequence having an identity of 80% or more with the nucleotide sequence of any one of SEQ ID NOs: 1352, 1372 and 1390, or
(d2) hybridizes under stringent conditions with a strand complementary to a nucleic acid consisting of the nucleotide sequence of any one of SEQ ID NOs: 1352, 1372 and 1390, and
(ii) a carrier therefor.

85. A composition comprising
(i) a nucleic acid selected from the group consisting of
(a) the nucleic acid of claim 65, wherein the SEQ ID NO is any one of 8, 21, and 36,
(b) a nucleic acid that
(b1) consists of a nucleotide sequence having an identity of 80% or more with the nucleotide sequence of any one of SEQ ID NOs: 1352, 1372, and 1390, or
(b2) hybridizes under stringent conditions with a strand complementary to a nucleic acid consisting of the nucleotide sequence of any one of SEQ ID NOs: 1352, 1372, and 1390,
(c) a nucleic acid consisting of a nucleotide sequence complementary to the nucleic acid of claim 65, wherein the SEQ ID NO is 1, and
(d) a nucleic acid that
(d1) consists of a nucleotide sequence having an identity of 80% or more to the nucleotide sequence of SEQ ID NO: 1337, or
(d2) hybridizes under stringent conditions with a strand complementary to a nucleic acid consisting of the nucleotide sequence of SEQ ID NO: 1337, and
(ii) a carrier therefor.

86. A composition comprising
(i) a double-stranded nucleic acid consisting of a
(A) a first strand nucleic acid selected from the group consisting of
(a) the nucleic acid of claim 65, wherein the SEQ ID NO is any one of 1, 8, 21, and 36,
(b) a nucleic acid that
(b1) consists of a nucleotide sequence having an identity of 80% or more to the nucleotide sequence of any one of SEQ ID NOs: 1337, 1352, 1372, and 1390, and
(c) hybridizes under stringent conditions with a strand complementary to a nucleic acid consisting of the nucleotide sequence of any one of SEQ ID NOs: 1337, 1352, 1372, and 1390,
(c) a nucleic acid consisting of a nucleotide sequence complementary to the nucleic acid of (a), and
(d) a nucleic acid consisting of a nucleotide sequence complementary to the nucleic acid of (b), and
(B) a second strand nucleic acid consisting of a nucleotide sequence complementary to the nucleotide sequence of the first strand nucleic acid, and
(ii) a carrier therefor.

87. A composition comprising
(i) a substance that promotes or suppresses
(A) the expression or function of a nucleic acid selected from the group consisting of
(a) the nucleic acid of claim 65, wherein the SEQ ID NO is any one of 1, 8, 21, and 36,
(b) a nucleic acid that
(b1) consists of a nucleotide sequence having an identity of 80% or more to the nucleotide sequence of any one of SEQ ID NOs: 1337, 1352, 1372, and 1390, and
(c) hybridizes under stringent conditions with a strand complementary to a nucleic acid consisting of the nucleotide sequence of any one of SEQ ID NOs: 1337, 1352, 1372, and 1390,
(c) a nucleic acid consisting of a nucleotide sequence complementary to the nucleic acid of (a), and
(d) a nucleic acid consisting of a nucleotide sequence complementary to the nucleic acid of (b), or
(B) the expression of a target gene of the nucleic acid of any one of (a) to (d), and
(ii) a carrier therefor.

88. A method of diagnosing a disease caused by a mast cell abnormality, which method comprises using the composition of claim 73 to diagnose a disease caused by a mast cell abnormality.

89. A method of treating a disease in a subject, which method comprises administering the composition of claim 73 to a subject suffering from a disease caused by a mast cell abnormality, thereby treating the disease caused by a mast cell abnormality in the subject.

90. A method of diagnosing a disease caused by a mast cell abnormality, which method comprises using the composition of claim 74 to diagnose a disease caused by a mast cell abnormality.

91. A method of treating a disease in a subject, which method comprises administering the composition of claim 74 to a subject suffering from a disease caused by a mast cell abnormality, thereby treating the disease caused by a mast cell abnormality in the subject.

92. A method of diagnosing a disease caused by a mast cell abnormality, which method comprises using the composition of claim 75 to diagnose a disease caused by a mast cell abnormality.

93. A method of treating a disease in a subject, which method comprises administering the composition of claim 75 to a subject suffering from a disease caused by a mast cell abnormality, thereby treating the disease caused by a mast cell abnormality in the subject.

94. A method of diagnosing a disease caused by an abnormality of mesenchymal stem cell proliferation and/or differentiation, which method comprises using the composition of claim 76 to diagnose a disease caused by an abnormality of mesenchymal stem cell proliferation and/or differentiation.

95. A method of treating a disease in a subject, which method comprises administering the composition of claim 76 to a subject suffering from a disease caused by an abnormality of mesenchymal stem cell proliferation and/or differentiation, thereby treating the disease caused by an abnormality of mesenchymal stem cell proliferation and/or differentiation in the subject.

96. A method of diagnosing a disease caused by an abnormality of mesenchymal stem cell proliferation and/or differentiation, which method comprises using the composition of claim 77 to diagnose a disease caused by an abnormality of mesenchymal stem cell proliferation and/or differentiation.

97. A method of treating a disease in a subject, which method comprises administering the composition of claim 77 to a subject suffering from a disease caused by an abnormality of mesenchymal stem cell proliferation and/or differentiation, thereby treating the disease caused by an abnormality of mesenchymal stem cell proliferation and/or differentiation in the subject.

98. A method of diagnosing a disease caused by a cell proliferation abnormality, which method comprises using the composition of claim 78 to diagnose a disease caused by a cell proliferation abnormality.

99. A method of treating a disease in a subject, which method comprises administering the composition of claim 78 to a subject suffering from a disease caused by a cell proliferation abnormality, thereby treating the disease caused by a cell proliferation abnormality in the subject.

100. The method of claim 99, wherein the disease caused by a cell proliferation abnormality is a disease selected from the group consisting of cancers, arteriosclerosis, rheumatoid arthritis, prostatic hyperplasia, blood vessel restenosis after percutaneous transvascular coronary angioplasty, fibroid lung, glomerulonephritis, and autoimmune diseases.

101. A method of suppressing cell proliferation, which method comprises using the composition of claim 78 as a cell proliferation suppressant to suppress cell proliferation.

102. A method of diagnosing a disease caused by a cell proliferation abnormality, which method comprises using the composition of claim 79 to diagnose a disease caused by a cell proliferation abnormality.

103. A method of treating a disease in a subject, which method comprises administering the composition of claim 79 to a subject suffering from a disease caused by a cell proliferation abnormality, thereby treating the disease caused by a cell proliferation abnormality in the subject.

104. The method of claim 103, wherein the disease caused by a cell proliferation abnormality is a disease selected from the group consisting of cancers, arteriosclerosis, rheumatoid arthritis, prostatic hyperplasia, blood vessel restenosis after percutaneous transvascular coronary angioplasty, fibroid lung, glomerulonephritis, and autoimmune diseases.

105. A method of suppressing or promoting cell proliferation, which method comprises using the composition of claim 79 as a cell proliferation suppressant or a cell proliferation promoter to suppress cell proliferation or to promote cell proliferation, respectively.

106. A method of promoting mast cell degranulation, which method comprises using the composition of claim 80 as a mast cell degranulation promoter to promote mast cell degranulation.

107. A method of suppressing mast cell degranulation, which method comprises using the composition of claim 81 as a mast cell degranulation suppressant to suppress mast cell degranulation.

108. A method of promoting or suppressing mast cell degranulation, which method comprises using the composition of claim 82 as a mast cell degranulation promoter or a mast cell degranulation suppressant to promote mast cell degranulation or to suppress mast cell degranulation, respectively.

109. A method of promoting or suppressing mast cell degranulation, which method comprises using the composition of claim 83 as a mast cell degranulation promoter or a mast cell degranulation suppressant to promote mast cell degranulation or to suppress mast cell degranulation, respectively.

110. A method of promoting mesenchymal stem cell proliferation, which method comprises using the composition of claim 84 as a mesenchymal stem cell proliferation promoter to promote mesenchymal stem cell proliferation.

111. A method of suppressing mesenchymal stem cell proliferation, which method comprises using the composition of claim 85 as a mesenchymal stem cell proliferation suppressant to suppress mesenchymal stem cell proliferation.

112. A method of promoting or suppressing mesenchymal stem cell proliferation, which method comprises using the composition of claim 86 as a mesenchymal stem cell proliferation promoter or a mesenchymal stem cell proliferation suppressant to promote mesenchymal stem cell proliferation or to suppress mesenchymal stem cell proliferation, respectively.

112. A method of promoting or suppressing mesenchymal stem cell proliferation, which method comprises using the composition of claim 87 as a mesenchymal stem cell proliferation promoter or a mesenchymal stem cell proliferation suppressant to promote mesenchymal stem cell proliferation or to suppress mesenchymal stem cell proliferation, respectively.

113. A method of detecting expression or a mutation of a nucleic acid, which method comprises using a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), to detect expression or a mutation of a nucleic acid of any one of (a) to (f).

114. A method of screening for a substance that promotes or suppresses expression or function of a nucleic acid, which method comprises using a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), to screen for a substance that promotes or suppresses expression or function of a nucleic acid of any one of (a) to (f).

115. A method of isolating a cell, which method comprises using a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), to isolate a cell that expresses a nucleic acid of any one of (a) to (f).

116. A method of suppressing expression of a target gene, which method comprises using a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), to suppress expression of a target gene of a nucleic acid of any one of (a) to (f).

117. A method of screening for a diagnostic reagent for a disease caused by a mast cell abnormality, which method comprises using promotion or suppression of expression or function of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), as an index to screen for a diagnostic reagent for a disease caused by a mast cell abnormality.

118. A method of screening for a diagnostic reagent for a disease caused by a mast cell abnormality, which method comprises using suppression of expression of a target gene of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), as an index to screen for a diagnostic reagent for a disease caused by a mast cell abnormality.

119. A method of screening for a mast cell degranulation promoter or a mast cell degranulation suppressant, which method comprises using promotion or suppression of
(i) the expression or function of a nucleic acid selected from the group consisting of
(a) the nucleic acid of claim 65, wherein the SEQ ID NO is any one of 1, 2, 3, 8, 14, 20, 22, 25, 32, and 36,
(b) a nucleic acid consisting of a nucleotide sequence having an identity of 80% or more to the nucleotide sequence of any one of SEQ ID NOs: 1337, 1338, 1339, 1352, 1363, 1371, 1373, 1377, 1386, and 1390, and
(c) a nucleic acid that hybridizes under stringent conditions with a strand complementary to a nucleic acid consisting of the nucleotide sequence of any one of SEQ ID NOs: 1337, 1338, 1339, 1352, 1363, 1371, 1373, 1377, 1386, and 1390, or
(ii) the expression of a target gene of the nucleic acid of (a), (b), or (c), as an index to screen for a mast cell degranulation promoter or a mast cell degranulation suppressant.

120. A method of screening for a diagnostic reagent for a disease caused by an abnormality of mesenchymal stem cell proliferation and/or differentiation, which method comprises using promotion or suppression of
(i) the expression or function of a nucleic acid selected from the group consisting of
(a) the nucleic acid of claim 65, wherein the SEQ ID NO is any one of 1, 8, 21, and 36,
(b) a nucleic acid consisting of a nucleotide sequence having an identity of 80% or more to the nucleotide sequence of any one of SEQ ID NOs: 1337, 1352, 1372, and 1390, and
(c) a nucleic acid that hybridizes under stringent conditions with a strand complementary to a nucleic acid consisting of the nucleotide sequence of any one of SEQ ID NOs: 1337, 1352, 1372, and 1390, or
(ii) the expression of a target gene of the nucleic acid of (a), (b), or (c), as an index to screen for a diagnostic reagent for a disease caused by an abnormality of mesenchymal stem cell proliferation and/or differentiation.

121. A method of screening for a mesenchymal stem cell proliferation promoter or a mesenchymal stem cell proliferation suppressant, which method comprises using promotion or suppression of
(i) the expression or function of a nucleic acid selected from the group consisting of
(a) the nucleic acid of claim 65, wherein the SEQ ID NO is any one of 1, 8, 21, and 36,
(b) the nucleic acid that
(b1) consists of a nucleotide sequence having an identity of 80% or more with the nucleotide sequence of any one of SEQ ID NOs: 1337, 1352, 1372, and 1390, or
(b2) hybridizes under stringent conditions with a strand complementary to a nucleic acid consisting of the nucleotide sequence of any one of SEQ ID NOs: 1337, 1352, 1372, and 1390,
(c) a nucleic acid consisting of a nucleotide sequence complementary to the nucleic acid of (a), and
(d) a nucleic acid consisting of a nucleotide sequence complementary to the nucleic acid of (b), or
(ii) the expression of a target gene of the nucleic acid of any one of (a) to (d), as an index to screen for a mesenchymal stem cell proliferation promoter or a mesenchymal stem cell proliferation suppressant.

122. A method of screening for a diagnostic reagent for a disease caused by a cell proliferation abnormality, which method comprises using promotion or suppression of
(i) the expression or function of
(a) the nucleic acid of claim 65, wherein the SEQ ID NO is any one of 1, 3, 8, 20, 21, 22, 32, and 36,
(b) a nucleic acid consisting of a nucleotide sequence having an identity of 80% or more to the nucleotide sequence of any one of SEQ ID NOs: 1337, 1339, 1352, 1371, 1372, 1373, 1386, and 1390, or
(c) a nucleic acid that hybridizes under stringent conditions with a strand complementary to a nucleic acid consisting of the nucleotide sequence of any one of SEQ ID NOs: 1337, 1339, 1352, 1371, 1372, 1373, 1386, and 1390, or
(ii) the expression of a target gene of the nucleic acid of (a), (b), or (c), as an index to screen for a diagnostic reagent for a disease caused by a cell proliferation abnormality.

123. A method of screening for a cell proliferation suppressant or proliferation promoter, which method comprises using promotion or suppression of
(i) the expression or function of
(a) the nucleic acid of claim 65, wherein the SEQ ID NO is any one of 1, 3, 8, 20, 21, 22, 32, and 36,
(b) a nucleic acid consisting of a nucleotide sequence having an identity of 80% or more to the nucleotide sequence of any one of SEQ ID NOs: 1337, 1339, 1352, 1371, 1372, 1373, 1386, and 1390, or
(c) a nucleic acid that hybridizes under stringent conditions with a strand complementary to a nucleic acid consisting of the nucleotide sequence of any one of SEQ ID NOs: 1337, 1339, 1352, 1371, 1372, 1373, 1386, and 1390, or
(ii) the expression of a target gene of the nucleic acid of (a), (b), or (c), as an index to screen for a cell proliferation suppressant or proliferation promoter.

124. A method of screening for a therapeutic agent for a disease caused by a mast cell abnormality, which method comprises using promotion or suppression of the expression or function of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), as an index to screen for a therapeutic agent for a disease caused by a mast cell abnormality.

125. A method of screening for a therapeutic agent for a disease caused by a mast cell abnormality, which method comprises using suppression of the expression of a target gene of a nucleic acid selected from the group consisting of
(a) the nucleic acid of claim 65,
(b) a nucleic acid that
(b1) consists of a nucleotide sequence having an identity of 80% or more to the nucleotide sequence of any one of SEQ ID NOs: 1337 to 2851, or
(b2) hybridizes under stringent conditions with a strand complementary to a nucleic acid consisting of the nucleotide sequence of any one of SEQ ID NOs: 1337 to 2851,
(c) a nucleic acid consisting of a nucleotide sequence complementary to the nucleic acid of (a),
(d) a nucleic acid consisting of a nucleotide sequence complementary to the nucleic acid of (b),
(e) a double-stranded nucleic acid consisting of the nucleic acid of (a) and (c), and
(f) a double-stranded nucleic acid consisting of the nucleic acid of (b) and (d), as an index to screen for a therapeutic agent for a disease caused by a mast cell abnormality.

126. A method of screening for a therapeutic agent for a disease caused by an abnormality of mesenchymal stem cell proliferation and/or differentiation, which method comprises using promotion or suppression of
(i) the expression or function of
(a) the nucleic acid of claim 65, wherein the SEQ ID NO is any one of 1, 8, 21, and 36,
(b) a nucleic acid consisting of a nucleotide sequence having an identity of 80% or more to the nucleotide sequence of any one of SEQ ID NOs: 1337, 1352, 1372, and 1390, or
(c) a nucleic acid that hybridizes under stringent conditions with a strand complementary to a nucleic acid consisting of the nucleotide sequence of any one of SEQ ID NOs: 1337, 1352, 1372, and 1390, or
(ii) the expression of a target gene of the nucleic acid of (a), (b), or (c), as an index to screen for a therapeutic agent for a disease caused by an abnormality of mesenchymal stem cell proliferation and/or differentiation.

127. A method of screening for a therapeutic agent for a disease caused by a cell proliferation abnormality, which method comprises using promotion or suppression of
(i) the expression or function of
(a) the nucleic acid of claim 65, wherein the SEQ ID NO is any one of 1, 3, 8, 20, 21, 22, 32, and 36,
(b) a nucleic acid consisting of a nucleotide sequence having an identity of 80% or more to the nucleotide sequence of any one of SEQ ID NOs: 1337, 1339, 1352, 1371, 1372, 1373, 1386, and 1390, or
(c) a nucleic acid that hybridizes under stringent conditions with a strand complementary to a nucleic acid consisting of the nucleotide sequence of any one of SEQ ID NOs: 1337, 1339, 1352, 1371, 1372, 1373, 1386, and 1390, or
(ii) the expression of a target gene of the nucleic acid of (a), (b), or (c), as an index to screen for a therapeutic agent for a disease caused by a cell proliferation abnormality.
